SEQUENCE LISTING (18 sequences)

SEQ ID NO: 1 - protein, 16 aa, synthetic construct
GCRDVPMSMRGGDRCG

SEQ ID NO: 2 - protein, 12 aa, synthetic construct
CGPQGIAGQGCR

SEQ ID NO: 3 - protein, 8 aa, synthetic construct
GPQGIAGQ

SEQ ID NO: 4 - protein, 8 aa, synthetic construct
GPQGIWGQ

SEQ ID NO: 5 - protein, 8 aa, synthetic construct
VPMSMRGG

SEQ ID NO: 6 - protein, 7 aa, synthetic construct
QPQGLAK

SEQ ID NO: 7 - protein, 9 aa, synthetic construct
GPLGLSLGK

SEQ ID NO: 8 - protein, 8 aa, synthetic construct
GPLGMHGK

SEQ ID NO: 9 - protein, 6 aa, synthetic construct
CGRGDS

SEQ ID NO: 10 - protein, 6 aa, synthetic construct
  VARIANT 3: note = Any amino acid
GFXGER

SEQ ID NO: 11 - protein, 5 aa, synthetic construct
IKVAV

SEQ ID NO: 12 - protein, 5 aa, synthetic construct
YIGSR

SEQ ID NO: 13 - protein, 7 aa, synthetic construct
MNYYSNS

SEQ ID NO: 14 - protein, 7 aa, synthetic construct
CNYYSNS

SEQ ID NO: 15 - protein, 4 aa, synthetic construct
DAPS

SEQ ID NO: 16 - protein, 6 aa, synthetic construct
AELDVP

SEQ ID NO: 17 - protein, 6 aa, synthetic construct
VALDEP

SEQ ID NO: 18 - protein, 6 aa, synthetic construct
NGRAHA

(19) United States
(12) Patent Application Publication
(10) Pub. No.: US 2026/0256842 A1
(43) Pub. Date: Sep. 3, 2026

(54) THYMIC CELL COMPOSITIONS AND METHODS OF MAKING

(71) Applicant: Thymmune Therapeutics, Inc., Cambridge, MA (US)

(72) Inventors: Bing Lim, Cambridge, MA (US); Stan Wang, Cambridge, MA (US); Behzad Gerami-Naini, Cambridge, MA (US); Qiumei Du, Cambridge, MA (US); Henry Thomas Beaman, Jr., Cambridge, MA (US); Benjamin A. Schwarz, Cambridge, MA (US)

(21) Appl. No.: 19/163,345

(22) PCT Filed: Apr. 4, 2024
(86) PCT No.: PCT/US2024/023120

§ 371 Date: Sep. 8, 2025

Related U.S. Application Data
(60) Provisional application No. 63/457,314, filed on Apr. 5, 2023.

(51) Int. Cl. A61K 35/26; A61P 37/06; C12N 5/078

(52) U.S. Cl. CPC A61K 35/26; A61P 37/06; C12N 5/065; C12N 2500/38; C12N 2501/117; C12N 2501/119; C12N 2501/15; C12N 2501/155; C12N 2501/2322; C12N 2501/25; C12N 2501/415; C12N 2506/45; C12N 2533/74

(57) ABSTRACT

The present disclosure provides thymic cells, thymic organoids, and animal models comprising such thymic cells and thymic organoids. It also provides methods for inducing differentiation of pluripotent stem cells into definitive endoderm (DE), anterior foregut endoderm (AFE), ventral pharyngeal endoderm (VPE), thymic epithelial progenitor (TEP) cells, thymic epithelial cells (TECs) and thymic organoids in vitro and in vivo.

### RELATED APPLICATIONS

This application claims priority under 35 U.S.C. § 119 to U.S. Provisional Application No. 63/457,314, filed on Apr. 5, 2023. The content of this application is incorporated herein by reference in its entirety.

### GOVERNMENT INTERESTS

This invention was made with Government support under Agreement No. AY2AX000004 awarded by the Advanced Research Projects Agency for Health (ARPA-H). The Government has certain rights in the invention.

### FIELD OF THE INVENTION

The present disclosure relates generally to bioengineering of pluripotent stem cells, and more specifically to bioengineered thymic cells, thymic organoids, humanized animal models comprising these thymic cells and thymic organoids, and their methods of use.

### BACKGROUND INFORMATION

The thymus is a primary lymphoid organ that plays a central role in the immune system. The microenvironment of the thymus provides a unique training ground for the development of maturation of effector cells such as lymphocytes (e.g., T cells). Complex interactions between thymic cells and effector cells can determine the phenotype and functionality of the effector cells. Some thymic cell-effector cell interactions are tuned such that recognition of factors expressed by the thymic cells promotes the survival of the effector cells. In contrast, other thymic cell-effector cell interactions may result in the death of the effector cells. By controlling such interactions, the thymus plays a pivotal role in establishing a repertoire of effector cells that are able to mount an activated immune response to foreign invaders while establishing tolerance to self.

Thymic cell-effector cell interactions occur within the intricate three dimensional network of the thymus which creates a complex microenvironment for effector cell development. There remains a need for thymic cell based therapies that recapitulate and mimic the native environment of the thymus.

### SUMMARY OF THE INVENTION

The present disclosure provides thymic cells, thymic organoids, and animal models comprising such thymic cells and thymic organoids. It also provides methods of making and maintaining thymic cells and thymic organoids in vitro and in vivo.

In particular, the present disclosure provides methods for inducing differentiation of pluripotent stem cells into thymic cells. Such methods can include the steps of encapsulating the pluripotent stem cells in a polymer, differentiating the pluripotent stem cells into definitive endoderm (DE) cells. DE cells can be cultured and differentiated into anterior foregut endoderm (AFE) cells by contacting or incubating the DE cells with BMP inhibitor, a TGFβ inhibitor, an FGF, an ascorbic acid and/or a combination thereof. AFE cells can be cultured and differentiated into ventral pharyngeal endoderm (VPE) cells by culturing in a first VPE medium, and/or a second VPE medium. The first VPE medium can include ascorbic acid, a retinoic acid, an FGF, and/or a TGFβ inhibitor. In some embodiments, first VPE media can further include a WNT inhibitor. The second VPE medium can include Noggin, a WNT activator, an FGF, a retinoic acid, and/or an ascorbic acid. In some embodiments, the second VPE media can further include a BMP inhibitor, an SHH inhibitor, or a combination thereof. The VPE cells can be further differentiated into thymic cells such as thymic epithelial progenitor cells (TEPs) by contacting or incubating the VPE cells with an ascorbic acid, an FGF, a BMP, and/or a WNT activator.

In one aspect, the polymer is alginate. In another aspect, the polymer comprises an alginate and gelatin A hydrogel.

In one aspect, the methods for inducing differentiation of pluripotent stem cells into thymic cells do not include an encapsulation step but do include the steps of: differentiating the pluripotent stem cells into definitive endoderm (DE) cells; culturing the DE cells and differentiating the DE cells into anterior foregut endoderm (AFE) cells by contacting or incubating the DE cells with a BMP inhibitor, a TGFβ inhibitor, an FGF, an ascorbic acid or a combination thereof; culturing the AFE cells and differentiating the anterior foregut cells into ventral pharyngeal endoderm (VPE) cells by: (i) contacting or incubating the AFE cells in a first VPE medium comprising ascorbic acid, a retinoic acid, an FGF, a TGFβ inhibitor or a combination thereof, and (ii) contacting or incubating the AFE cells in a second VPE medium comprising a Noggin, a WNT activator, an FGF, a retinoic acid, an ascorbic acid, or a combination thereof; and culturing the VPE cells and differentiating the VPE cells into thymic cells, by contacting or incubating the VPE cells with an ascorbic acid, an FGF, a BMP, a WNT activator or a combination thereof; wherein the thymic cells are thymic epithelial progenitors (TEPs) and/or thymic epithelial cells (TECs).

In some embodiments, the thymic cells can be thymic epithelial progenitors (TEPs) or thymic epithelial cells (TECs). TEPs can be further differentiated into TECs by culturing the TEPs with an Interleukin, a WNT activator, a RANKL, an FGF, a BMP, and/or an ascorbic acid.

In some embodiments, pluripotent stem cells can be differentiated to DE cells by contacting or culturing the pluripotent stem cells in a first growth medium, containing Activin A, PI-103, and/or CHIR99021. The differentiation to DE cells can further include culturing cells in a second growth medium containing Activin A, a BMP inhibitor, PI-103, and/or CHIR99021.

In some embodiments, BMP inhibitor can be LDN193189. In some embodiments, the TGFβ inhibitor can be SB431542. In some embodiments, FGF can be FGF8b, FGF7, FGF10, FGF1, bFGF or a combination thereof. In some embodiments, the WNT activator can be CHIR99021. In some embodiments, the BMP can be BMP2, BMP4 or a combination thereof. In some embodiments, the interleukin can be IL22. In some embodiments, the WNT inhibitor can be IWR-1. In some embodiments, the BMP inhibitor can be LDN193189. In some embodiments, the SHH inhibitor can SANT-1.

Pluripotent stem cells, the DE cells, the AFE cells, the VPE cells or thymic cells of the disclosure can be cultured in suspension. In some embodiments, the pluripotent stem cells, the DE cells, the AFE cells, the VPE cells and the thymic cells can be cultured as aggregates in suspension. In other embodiments, the pluripotent stem cells, the DE cells, the AFE cells, the VPE cells, and the thymic cells can be cultured as single cells in suspension. In some additional embodiments, one or more of the following cell types can be cultured as single cells in suspension: pluripotent stem cells, DE cells, AFE cells, VPE cells, and thymic cells.

In some embodiments, the pluripotent stem cells, the DE cells, the AFE cells, the VPE cells or thymic cells can be attached to a solid substrate. In some embodiments, the pluripotent stem cells, the DE cells, the AFE cells, the VPE cells or thymic cells can be attached to a solid substrate that includes an extracellular matrix-based medium.

In some other embodiments, pluripotent stem cells and DE cells can be cultured as aggregates in suspension, then the resulting DE aggregates can be attached to a solid substrate that includes an extracellular matrix-based medium and further differentiated into AFE, VPE and TEP cells via two-dimensional (2D) adherent cultures. In some additional embodiments, the pluripotent stem cells are human induced pluripotent stem cells. In some other additional embodiments, the human induced pluripotent stem cells are encapsulated. In still other additional embodiments, the human induced pluripotent stem cells are not encapsulated.

The methods of the disclosure can be performed for about 15 days to 30 days. In some embodiments, the methods can be performed for about 18 days to 25 days. In some embodiments, the pluripotent stem cells can be differentiated into definitive endoderm cells for about 5 days. In some embodiments, the DE cells can be differentiated into AFE cells for from about 2 days to about 3 days. In some embodiments, the AFE cells can be differentiated into VPE cells in the first VPE media for about 2 to 4 days and in the second VPE media for from about 2 days to 3 days. In some embodiments, the VPE cells can be differentiated into thymic cells for about 3 days to 12 days.

The present disclosure also provides thymic cells e.g., TEPs and TECs generated by the methods described herein.

Also provided herein are methods for culturing thymic cells in vitro. Such methods can include culturing or incubating the thymic cells in a thymic cell medium that includes FGF10, BMP4, FGF8b, CHIR99021, and/or ascorbic acid. In some embodiments, the thymic cell medium can further include FGF7 and RANKL. The methods can also include culturing thymic cells in suspension. As a non-limiting example, the thymic cells can be cultured as aggregates in suspension.

In some embodiments, the present disclosure provides methods of increasing FOXN1 expression in a population of thymic cells. Such methods can include freezing the population of thymic cells, thawing the population of thymic cells, and measuring and comparing the expression of FOXN1 in the population of thymic cells prior to freezing and comparing with FOXN1 expression after thawing the population of thymic cells. In some embodiments, the expression of FOXN1 can be increased by from about 10-fold to 100 fold. In some embodiments, the expression of FOXN1 can be increased by about: 2-fold, 3-fold, 4-fold, 5-fold, 6-fold, 7-fold, 8-fold, 9-fold, or 10-fold. In some embodiments, the expression of FOXN1 can be increased by about: 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In some embodiments, the population of thymic cells can be cultured as aggregates in suspension.

Also provided herein are pharmaceutical compositions that include a population of thymic cells prepared by the methods described herein. The present disclosure also provides methods of treating or preventing a condition in a subject by administering the pharmaceutical compositions of the disclosure. In some embodiments, the condition is associated with an absence, decline, or aberrant functioning of the thymus in the subject. The condition can be an immunodeficiency, a cancer, an autoimmune disease, an infectious disease, or graft versus host disease (GvHD). In some embodiments, the pharmaceutical compositions are administered parenterally. For example, the compositions can be implanted or injected into one or more lymph nodes of the subject.

The present disclosure provides compositions. The compositions may include a population of thymic cells. In some embodiments, the population of thymic cells may include one or more cell types. The cell types may be selected from one or more of the following: iTEC, mTECs, comeocyte-like mTEC, cTEC-high, cTEC-low, mTEC-low. The compositions may also include a thymic support system (TSS).

In some embodiments, the population of thymic cells may be prepared by the differentiation of iPS cells to thymic cells.

In some embodiments, TSS may include a polymer. The polymer may be a biogenic polymer or a synthetic polymer. Biogenic polymers may be polypeptide-based biogenic polymers, polynucleotide-based biogenic polymers, polysaccharide-based polymers, or a combination thereof. Non-limiting examples of polypeptide-based polymers include collagen, fibrin, fibrinogen, gelatin, silk, elastin, myosin, keratin, and actin. Non-limiting examples of polysaccharide-based include alginate, chitin, chitosan, hyaluronic acid, cellulose, agarose, starch, cellulose, dextran, hyaluronic acid, glycogen and glycosaminoglycans.

In some embodiments, the polymer may be synthetic polymer. Non-limiting examples of synthetic polymers include polycaprolactone, polyglycolic acid, poly lactic acid, polylactic-co-glycolic acid, poly(ethylene oxide) polyethylene glycol, polyurethane,), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), polyhydroxybutyrate (PHB), Polypropylene fumarate (PPF), a polyvinyl alcohol (PVA), a polypropylene carbonate, a polyanhydride, a polyphosphazene, a polygermane, a polyorthoester, a polyester, a polyamide, a polyolefin, a polycarbonate, a polyaramide, a polyimide,), chitosan, Poly(2-hydroxyethyl methacrylate) (PHEMA), 2-Hydroxyethyl methacrylate (HEMA), Hydroxyethoxyethyl metha-crylate (HEEMA), Hydroxydiethoxy ethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic poly allyl ammonium chloride, and/or poly(N-isopropyl acrylamide) (PNIPAM).

Polymers of the disclosure may form a hydrogel. In some embodiments, the polymers may be cross-linked.

The TSS of the disclosure may further include an extracellular matrix component and/or an agent. In some embodiments, the extracellular matrix component may be an extracellular matrix protein, or a region or a portion thereof. Non-limiting examples of extracellular matrix protein include fibronectin, laminin, vitronectin, tenascin, entactin, thrombospondin, elastin, gelatin, collagen, fibrin, merosin, ankaline, chondronectin, link protein, bone sialo acid protein, osteocalcin, osteopontin, epinectin, hyaluronectin, unjulin, epiligrin or carinine. In some embodiments, the extracellular matrix component may be a peptide derived from the extracellular matrix protein. Non-limiting examples of the peptide include an amino acid sequence of any of SEQ ID NO: 9-18.

The TSSs of the disclosure may include a biological agent or a chemical agent. In some embodiments, the biological agent may be a ligand, an immune modulator, or a hormone.

Compositions of the disclosure may further include supporting cells, stem cells and/or effector cells. Non-limiting examples of supporting cells include myelin cells, myoid cells, neuroendocrine cells, tuft cells, ionocytes, endothelial cells, mesenchymal stem cells, or fibroblasts.

Also provided herein are methods of treating or preventing a condition in a subject by administering the compositions described herein.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 is a graph showing expression of TEP markers in cells differentiated by encapsulation of iPSC cells compared to cells differentiated in 2 dimensional cultures as well as cells in 2 dimensional culture and subjected to a freeze and thaw.

FIGS. 2A-2B are a schematic and bar graph that show the timing, process and results for differentiation of chitosan-coated alginate encapsulated hiPSC aggregates into thymic epithelial cells. FIG. 2A is a schematic illustrating the process for differentiation of chitosan-coated alginate encapsulated hiPSC aggregates into thymic epithelial cells (TEC, also referred to as “THY-100”) using 3D suspension culture. The cells were encapsulated in alginate (via extrusion or via emulsification) and cultured in 3D suspension at 45 rotations per minute (RPM) at 37° C. in 5% CO₂. FIG. 2A also shows the differentiation stages which take place from Day 0 to Day 21, representing Stage 0 through Stage 4 when the thymic epithelial progenitors (TEPs) are frozen (indicated by the dashed line on the right side); it also shows a final differentiation stage (Stage 5) that takes place after the TEP cells are thawed. The photographs beneath the ovals in FIG. 2A show the hiPSC cells after encapsulation (far left image) and at each stage from Stage 0 to Stage 5 (far right image). All images were taken at 10× magnification, and the scale bar in each image represents 100 um.

FIG. 2B is a bar graph which shows the amount of FOXN1 that was expressed by the Stage 5 TECs after they had been thawed and cultured under 3D suspension in TEC Freeze/Thaw media for 7 days. This expression data was obtained using qRT-PCR, with FOXN1 expression levels normalized to GAPDH expression and the cell concentration at 200,000 cells/mL. The FOXN1 expression level was about five-fold higher than the FOXN1 expression level measured at day 23 (before the TEC FT media was added to the cells and before they were frozen in liquid nitrogen and stored at −80° C.).

FIGS. 3A-3N show flow cytometry results characterizing hiPSC-derived TECs. FIGS. 3A to 3N present the results for the first of four stains.

FIGS. 4A-4M show flow cytometry results characterizing hiPSC-derived TECs. FIGS. 4A to 4M present the results for the second of four stains.

FIGS. 5A-5O show flow cytometry results characterizing hiPSC-derived TECs. FIGS. 5A to 5O present the results for the third of five stains.

FIGS. 6A-6F show flow cytometry results characterizing hiPSC-derived TECs. FIGS. 6A to 6F present the results for the fourth of four stains.

FIG. 7 is a bar graph illustrating the expression levels for various genes for alginate-encapsulated hiPSC-derived TEC cells on Day 23, prior to freezing (see left-most columns, “AEMF-200K-090123 TEC”). The expression levels were also assessed for a sample of the same cells on Day G, (after having been frozen, thawed and grown for 7 days in TEC FT media); these results are shown in the columns on the right (“AEMF-200K-090123-FT1”). Both the TEC and FT1 samples had a cell concentration of 200,000 cells/mL. These results demonstrate that differentiated TECs display thymopoietic capacity for supporting T-cell development in vitro.

FIGS. 8A-8N is a set of graphs and flow cytometry contour maps showing human iPS-derived TEPs can efficiently induce the development of human T cells in vivo.

FIGS. 9A-9G is a set of schematics and graphs illustrating hiPSC to TEC differentiation using non-encapsulated single cells cultured using 3D suspension. FIGS. 9A to 9G illustrate representative hiPSC to TEC differentiation experiments using non-encapsulated single cells cultured in 3D suspension (FIG. 9A to 9C) as described in Example 5, or non-encapsulated single cells cultured in 3D in a bioreactor (FIG. 9D to 9F) using the same buffers as described in Example 5. Successful growth and differentiation to thymic epithelial precursor cells was achieved, as qualitatively shown in the representative micrographs of FIG. 9A (micrographs taken at 10× magnification; scale bar=100 μm) and FIG. 9B (micrograph taken at 10× magnification showing thymic cells at Stage 5; scale bar=100 μm). FIG. 9C is a bar graph illustrating the expression of FOXN1 in vitro by thymic cells produced as described in Example 5 (after the iPSC-derived TEP cells had been frozen, thawed and grown for 7 days in TEC FT media). The level of FOXN1 expression (which ranged from 0.0019 to 0.0022) was determined using qRT-PCR on samples at a concentration of about 200,000 thymic cells per mL, with FOXN1 expression normalized to GADPH expression. These results demonstrate that thymic cells which were differentiated as single cells in 3D suspension produced higher levels of FOXN1 in vitro as compared to thymic cells produced using other methods described herein. The results obtained when hiPSCs were differentiated as single cells in 3D in a bioreactor according to the process illustrated in FIG. 9D were also above the threshold of 0.0001 necessary to obtain sufficient T cell production in vivo once the thymic cells were transplanted into mice as in Example 6. FIG. 9G is a schematic that shows multiple attempts were made to get single cells, at various stages of differentiation (ranging from iPS through TEP) to differentiate in 3D suspension culture without encapsulation. All experiments in FIG. 9G were conducted with standard media conditions. Aggregated or dissociated single cells were cultured in 3D suspension at the indicated stages of differentiation. For Experiment 78B only, B27 was added to the cell culture media at the DE stage. Successful growth and differentiation are indicated by a dotted pattern. Cell death is indicated by diagonal lines.

FIGS. 10A-10D. FIG. 10A is a set of two schematics and six graphs illustrating the process, timing and results when non-encapsulated hiPSC aggregates are differentiated into TECs using a hybrid (2D/3D) differentiation protocol and transplanted into humanized NSG MHC-I/II double knock out mice. FIG. 10A is a schematic illustrating a hybrid (2D/3D) process for differentiation of non-encapsulated hiPSC aggregates into thymic epithelial cells (TEC, also referred to as “THY-100”). In this hybrid process, non-encapsulated hiPSCs were grown through the DE stage in 3D suspension culture at 45 rotations per minute (RPM) at 37° C. in 5% CO₂, then the DE aggregates were plated on Matrigel coated 24-well culture plates. The remaining differentiation stages (AFE, VPE and TEP) were all conducted in 2D adherent culture. FIG. 10A also shows the differentiation stages which take place from Day 0 to Day 21, representing Stage 0 through Stage 4 when the thymic epithelial progenitors (TEPs) are frozen (indicated by the dashed blue line on the right sides); it also shows a final differentiation stage (Stage 5) that takes place after the TEP cells have been frozen at −80° C. and thawed. The images beneath the ovals in FIG. 10A show the hiPSC cells at each stage from pre-Stage 0 (far left) to Stage 0 (2^{nd} image from left) through Stage 5 (far right image). All images were taken at 10× magnification, and the scale bar inside all images represents 100 um. FIG. 10B is a schematic illustrating the same hybrid (2D/3D) differentiation process, with the markers for specific differentiation stages indicated in the boxes beneath the ovals. FIGS. 10C-10D are graphs representing T cell development in humanized NSG MHC-I/II double knock out mice (Jax) transplanted with hiPS-derived TEPs differentiated using the hybrid (2D/3D) differentiation process described in FIG. 10A and FIG. 10B. The vertical axes of the three graphs in FIG. 10C show the TCRαβ-positive T cells (far left graph), CD4+T cells (middle graph), and CD8+ T cells (far right graph) expressed as a percentage of the total number of human CD45-positive cells (hCD45+); the horizontal axis represents the time points for sample collection (weeks post transplantation). The results shown in FIG. 10C are representative for the results obtained when the TEPs expressed FOXN1 in vitro at a level higher than the 0.0001 threshold that was found to be necessary for obtaining sufficient T cells in vivo. Specifically, the TEPs in FIG. 10C expressed FOXN1 at a level that was 8-fold higher than threshold (0.0001). The results shown in FIG. 10D are representative for the results obtained when the TEPs to be transplanted expressed FOXN1 in vitro at a level that was less than the 0.0001 threshold necessary to obtain sufficient T cells in vivo (0.00009). FOXN1 expression levels in vitro were determined using qRT-PCR normalized to GADPH expression. Each line (180-184 in FIG. 10C and 190-194 in FIG. 10D) represents a different mouse.

### DETAILED DESCRIPTION

### Introduction

Thymic epithelial cells are important in T cell differentiation. Thymic cells prepared as described herein may permit exploitation of thymus tissue and thymus cell properties, e.g., thymus-related immune functions, for therapeutic applications. For example, it is known that age-related decline in immune function is caused by changes in the composition and functional capabilities of thymic cells. In addition, changes in sex hormones, including androgens and estrogens, cause the thymus itself to atrophy or become senescent. The onset of thymic atrophy may begin as early as the onset of puberty. Thus, regeneration of thymic epithelial cells may provide for compositions and methods that mitigate age-related decline in immune function.

The complexity and compartmentalization of the thymic tissue allows the definition of distinct microenvironments with specific cues that direct effector cell development. Fetal thymus organ cultures (FTOC) or reaggregated thymus organ cultures (RTOC) have been developed as platforms to recreate niche environments that attempt to recapitulate thymic environments. In FTOC, the whole thymus is placed over a membrane that lies on top of a medium-embedded sponge (G. Anderson, et al., Cold Spring Harb. Protoc. 2007, 2007; the contents of which are herein incorporated by reference in its entirety). In the case of RTOC, after dissociation of the thymus, the cells of interest are selected by cell sorting technologies and are placed as a concentrated drop on top of a membrane, at the air-liquid interphase. (E. J. Jenkinson, et al., J. Exp. Med. 1992, 176, 845; the contents of which are herein incorporated by reference in their entirety). Although these methodologies reflect effector cell development during fetal ontogeny, compositions that can mimic the native environment and allow the generation of effector cells, especially in adults are still needed. The present disclosure provides thymic cells based compositions that combine thymic cells with thymic support systems to create microenvironments that mimic the thymus. Inventors have made the discovery that encapsulating iPS cells prior to differentiation with the differentiation protocols described herein can result in thymic cells expressing FOXN1 among other markers. Applying the thymic cell differentiation protocol in suspension, provides opportunities to scale up manufacturing of thymic cells for therapeutic applications.

### Compositions

### Cells

Cells of the disclosure may include, without limitation, thymic cells, effector cells, pluripotent stem cells, populations thereof and cells derived therefrom.

In some embodiments, cells of the present disclosure may be autologous, allogeneic, syngeneic, or xenogeneic in relation to a particular individual or subject. In some embodiments, the thymic cells may be autologous, allogeneic, syngeneic, or xenogeneic in relation to subjects ultimately benefiting from their clinical application. In some embodiments, the cells of the disclosure may be mammalian cells, particularly, human cells. Cells may be primary cells or immortalized cell lines. In some aspects, the cells of the disclosure may be prepared or derived from syngeneic cell sources. Any of the cells described herein may be characterized by markers known in the art for that cell type.

One or more cells of the disclosure may be engineered to ectopically express a polynucleotide. The polynucleotide may encode a polypeptide of interest. In some embodiments, the polypeptide of interest may be operably linked to an inducible element and/or an inducible promoter such that the expression of the polypeptide of interest is controlled by the inducible element and/or promoter. In some embodiments, the inducible element may include a ligand binding domain, e.g., ligand binding domain of FKBP, cyclophilin receptors, steroid receptors, cyclophilin receptor, and/or tetracycline receptor. Any of the compositions or methods of engineering cells described in International Patent Publication WO2021150837 may be useful in the present disclosure (the contents of which are herein incorporated by reference in their entirety). In some embodiments, the polypeptide of interest may be a cell death-inducing polypeptide such that the engineered cells are eliminated or killed when induced. Exemplary cell death-inducing polypeptides include, but are not limited to, Casp2, Casp3, Casp8, Casp9, Casp10, p53, BAX, DFF40, HSV-TK, and/or cytosine deaminase proteins. Any inducible promoter known in the art may also be useful in the present disclosure.

In some embodiments, cells of the disclosure may be engineered to ectopically express a protein which activates one or more checkpoint pathways to induce host immune cell exhaustion and anergy of the host cells to the cells of the disclosure. For example, the cells may be engineered to express an immune checkpoint protein according to the methods described in European Patent Publication EP3886759A1, the contents of which are herein incorporated by reference in their entirety. The immune checkpoint protein may be PD-1, PD-L1, PDL-2, CD47, CD39, CD73, CD200, HVEC, CEACAM1, CD155, TIM-3, LAG-3, CTLA-4, A2AR, B7-H3, B7-H4, HLA-E, BTLA, IDO, KIR, VISTA, or a combination thereof.

Cells of the disclosure may be organized into structures that resemble cylinders, rods, strings, filaments, or networks as described in US Patent Publication US20210213171, the contents of which are herein incorporated by reference in their entirety. Such architecture can lead to enhanced integration of the compositions of the disclosure into host organisms as evidenced by improved blood supply, and/or improved vasculature. The cells of the disclosure may be organized in clusters and/or islands embedded in ECM components.

In one embodiment, the compositions include thymic cells, effector cells, and one or more supporting cells such as mesenchymal stem cells. The compositions are maintained or cultured in the presence of one or more agents such as, but not limited to, chemokines. Non-limiting examples of chemokines include CXCL12 and/or CCL2.

### Thymic Cells

Compositions described herein may include a population of thymic cells. A thymic cell may be a cell with one or more phenotypic or genotypic markers associated with a cell derived from the thymus or a cell destined to become a cell of the thymus. In some embodiments, the population of thymic cells may be derived by the differentiation of pluripotent stem cells. In some embodiments, the pluripotent stem cells may be iPSCs.

In some embodiments thymic cells may be prepared from the differentiation of pluripotent stem cells that differentiate into thymic stems via one or more of the following steps: PSCs can differentiate and/or can be induced to differentiate into cells resembling the definitive endoderm (DE). Definitive endoderm cells can differentiate and/or can be induced to differentiate into cells resembling the third pharyngeal pouch endoderm (PPE) which are also referred to as ventral pharyngeal endoderm (VPE). Definitive endoderm cells can differentiate and/or be induced to differentiate into cells resembling the anterior foregut endoderm (AFE). AFE can differentiate and/or be induced to differentiate into cells resembling the third pharyngeal pouch endoderm (PPE) which are referred to as ventral pharyngeal endoderm (VPE). Thymic epithelial progenitor cells (TEPCs) can be generated from PPE cells. TECs can be derived from TEPCS. Each of the cell types described herein may be characterized by one or more markers. In some embodiments, pluripotent stem cells may be associated with the increased expression of markers such as, but not limited to, OCT4, SOX2, and/or NANOG. In some embodiments, definitive endodermal cells may be associated with the increased expression of markers such as, but not limited to, SOX17, FOXA2, CXCR4, and/or CERI. In some embodiments, anterior foregut cells (AFE) may be associated with the increased expression of markers such as, but not limited to, FOXA2, SOX2, and/or PAX9. In some embodiments, the third pharyngeal pouch endodermal cells may be associated with the increased expression of markers such as, but not limited to, HOXA3, TBX1, PAX9, EYA1, SIX1, PBX1, and/or PAX1. In some embodiments, thymic epithelial progenitor cells may be associated with the increased expression of markers such as, but not limited to FOXN1, K5, K8, and/or HOXA3. In some embodiments, the thymic cells may be derived from a DE cell, a third PPE cell, an AFE cell, a TEPC, and/or a TEC cell.

For the preparation of the population of thymic cells of the disclosure, pluripotent stem cells may be cultured and differentiated to definitive endoderm cells. The definitive endoderm cells may be further cultured and differentiated into anterior foregut cells. In some embodiments, the anterior foregut cells may be cultured and differentiated into pharyngeal endoderm cells. In some embodiments, the pharyngeal endoderm cells may be cultured and differentiated into, thymic cells, e.g., thymic epithelial cells. In some embodiments, the differentiation may be performed from about 14 days to about 21 days.

In some embodiments, thymic cells may be prepared from PSCs. In this regard, the methods may comprise culturing the pluripotent stem cells for a period of time and under conditions sufficient to differentiate the pluripotent stem cells into thymic cells. For example, the method may comprise culturing the pluripotent stem cells in the presence of the factors and/or inhibitors that drive the differentiation of PSCs to thymic cells. In some embodiments, these factors and/or inhibitors may comprise or consist of a pluripotent stem cell-specific inhibitor that selectively eliminates human pluripotent stem cells. In some embodiments, the pluripotent stem cell-specific inhibitor comprises or consists of an oleate synthesis inhibitor. In some embodiments, the pluripotent stem cell-specific inhibitor comprises or consists of an inhibitor of stearoyl-coA desaturase (SCD1) that inhibits the activity of SCD1 in human pluripotent stem cells. In some embodiments, the pluripotent stem cell-specific inhibitor comprises or consists of a derivative of N-acyl phenylhydrazine that comprises a phenylhydrazine (Ph-N[H,C]—NH) moiety. In some embodiments, the pluripotent stem cell-specific inhibitor comprises or consists of N′-phenylpyridine-4-carbohydrazide (NSC 14613, also known as PluriSIn #1).

In some embodiments, the methods for differentiating PSCs into thymic cells may be any methods known in the art. The methods for differentiating PSCs into thymic cells may include the use of one or more parameters known in the art for differentiation or combinations thereof. The parameters include, but are not limited to, (i) factors promoting differentiation (ii) inhibitors promoting differentiation (iii) duration of time for promoting differentiation (iv) temperature (v) substrate and/or (vi) supporting cells that promote differentiation. Any of the methods or parameters for differentiating PSCs into thymic cells described in following references may be used herein and include Parent et al. Cell Stem Cell. 2013 Aug. 1; 13(2):219-29; Soh et al. Stem Cell Rep. 2014 Vol. 2 j 925-937; Sun et al. Cell Stem Cell. 2013 Aug. 1; 13(2):230-6; Okabe et al. Cell. Reprog. 2015 Vol 17, No. 5; Su et al. Sci. Rep. 2015 5, 9882; Otsuka et al. Sci Rep 2020 10:224; International Patent Publications, WO2019060336, WO2020205859, WO2020220040, WO2014134213, WO2010143529, WO2011139628; WO2022076751, WO2014134213, WO2021222297 and Chinese Patent Publication CN201110121243; the contents of each of which are herein incorporated by reference in their entirety.

The thymic cells may be an embryonic, a fetal or an adult thymus.

In some embodiments, the population of thymic cells may include one or more cell types. The cell types may be iTEC, mTECs, Corneocyte-like mTEC, cTEC-high, cTEC-low, mTEC-low cell.

In some embodiments, the population of thymic cells may include thymic epithelial cells (TECs) In some embodiments, the TECs may be derived by the differentiation of iPSCs. During embryonic development TECs may be derived from non-hematopoietic cells which are negative for CD45 expression and positive for epithelial marker EpCAM. TECs may be cortical thymic epithelial cells (cTECs) and/or medullary thymic epithelial cells (mTECs). mTECs are characterized by cytokeratin 5 (KRT5 or K5) and cytokeratin 14 (KRT14 or K14) expression but low level of cytokeratin 8 (KRT8 or K8) expression, whereas cTECs express K8 and K18. In some embodiments, thymic cells may be derived from TECs that express both K5 and K8 (K5+K8+), which is typical in TECs cells found at the cortico-medullary junction. In some aspects, K5+K8+ cells may be progenitors for mTECs and/or cTECs. mTECs may also be positive for the expression of Ulex europaeus agglutinin-1 (UEA-1) on cell surface, but not Ly51 (e.g., UEA-1+Ly51−), while cTECs may be UEA-1-Ly51+.

In some embodiments, the population of thymic cells may include medullary thymic epithelial cells (mTECs). In some embodiments, the mTECs may be derived by the differentiation of iPSCs. In some embodiments, thymic cells may be or may be derived from mTECs. In some embodiments, mTECs may have high expression of markers such as, but not limited to, cytokeratin 5, cytokeratin 14, UEA-1, CD80, Cathepsin L, and/or Cathepsin S.

In some embodiments, thymic cells may be or may be derived from cTECs that have high expression of markers such as, but not limited to, cytokeratin 8, cytokeratin 18, Ly51, CD205, Cathepsin L and/or thymus-specific serine proteases. In some embodiments, the population of thymic cells may include cortical thymic epithelial cells (cTECs). cTECs are responsible for commitment to the T cell lineage and positive selection of early thymocytes. In some embodiments, the cTECs may be derived by the differentiation of iPSCs. As a non-limiting example, thymic cells may be or may be derived from cTECs that express markers such as: CCL25, and/or K5.

In some embodiments, thymic cells may be, or may be derived from, TECs that express one or more markers such as, FOXN1, PAX9, PAX1, DLIA, ISL1, EYA1, SIX1, IL7, K5, K8 and AIRE.

Thymic cells may be or may be derived from any of the cell types described by Park et al. 2020 Science Vol. 367, Issue 6480 (the contents of which are herein incorporated by reference in its entirety). For example, thymic cells may be derived from myoid cells, e.g., MYOD1 and MYOG expressing myoid cells (herein referred to as TEC(myo)) and/or from NEUROD1, SYP, CHGA-expressing TECS (herein referred to as TEC(neuro).

In some embodiments, the population of thymic cells may include any cell type described by Bautista et al. 2021 Nat Commun 12, 1096; the contents of which are herein incorporated by reference in its entirety. In some embodiments, the population of thymic cells derived by the differentiation of iPSCs may include “cTEC 10” cells described by Bautista et al. 2021 and may be characterized by lower levels of functional genes (HLA class II) and containing more K167⁺-proliferating cells. Thymic cells may be derived from “mTEC 10” cells described by Bautista et al. 2021 and characterized by the expression of CLDN4, lower levels of HLA class II, expression of PSMB11, PRSS16, CCL25, and high levels of chemokine CCL21.

In some embodiments, the population of thymic cells derived by the differentiation of hiPSCs or iPSCs may include any cell type described US20230159887, the contents of which are herein incorporated by reference in its entirety.

In some embodiments, the population of thymic cells derived by the differentiation of iPSCs may include “mTEC hi” cells described by Bautista et al. 2021 and characterized by SPIB, AIRE, FEZF2, higher levels of expression of HLA class II. Thymic cells may be or may be derived from comeocyte-like mTECs described by Bautista et al. 2021 and characterized by the expression of KRT1, and/or IVL.

In some embodiments, the population of thymic cells derived by the differentiation of iPSCs may include immature TEC (iTEC) described by Bautista et al. 2021, which express canonical TEC identity genes e.g., FOXN1, PAX9, SIX1.

In some embodiments, the population of thymic cells derived by the differentiation of iPSCs may include cTEC^{hi} (or cTEC high) cells which may be characterized by the expression of cell surface markers such as, but not limited to CTSV, SLC46A2, HLA-DMA, CXCL12, THY1, ENO1, CALR, ALCAM, ATPIF1, and/or HSPA5.

In some embodiments, the population of thymic cells derived by the differentiation of iPSCs may include AIRE+mTEC high cells which may be characterized by the expression of one or more markers such as, but not limited to LTF, HLA-DRA, CD74, HLA DRB1, HLA-DPA, HLA-DPB1, IL2RG, and/or FCER2.

In some embodiments, the population of thymic cells derived by the differentiation of iPSCs may include TECs that express one or more markers, such as, but not limited to KRT5, KRT8, AIRE, PSMB11, and/or PRSS16.

In some embodiments, the population of thymic cells derived by the differentiation of iPSCs may include TECs that express one or more markers, such as, but not limited to AIRE, CK5, CK8, CXCL12, CCL25, DLL4, and/or HLA-DR.

In some embodiments, the population of thymic cells derived by the differentiation of iPSCs may include corneocyte like mTECs. The cells are called comeocyte-like because they express genes such as keratin cytoskeletal 1 (KRT1), KRT10, SPINKS that are also expressed in comeocytes (terminally differentiated keratinocytes) of the skin. Corneocyte-like cells also express transcripts that overlap with mTEC, such as AIRE. Hence, they are referred to as Corneocyte-like mTEC. They are likely the precursors that give rise to Hassall's' corpuscles, a unique cell in the human thymus. It is also thought that these cells are derived from mTEC precursor cells (Noam Kadouri et al Nature Review Immunology 2020 v20:239; the contents of which are herein incorporated by reference in its entirety).

### Pluripotent Stem Cells (PSCs)

In some embodiments, the cells of the present disclosure may be derived from pluripotent stem cells.

Pluripotent stem cells have the capacity to give rise to any of the three germ layers: endoderm, mesoderm, and ectoderm. Pluripotent stem cells may comprise, for example, stem cells, e.g., embryonic stem cells, nuclear transfer derived embryonic stem cells, induced pluripotent stem cells (iPSC). The pluripotent stem cells may have a stem cell phenotype including (i) the ability to self-renew and (ii) pluripotency. Pluripotency-associated genes may include, but are not limited to, Oct-3/4, Sox2, Nanog, GDF3, REXI, FGF4, ESGI, DPPA2, DPPA4, hTERT and SSEAI.

Cells described herein may be derived from embryonic stem cells. ES cells may include a cell that (a) self-renews (b) differentiates to produce all cell types in an organism and/or (c) is derived from a developing organism. ES cells may be derived from the inner cell mass of the blastula of a developing organism. ES cells may also be derived from the blastomere generated by single blastomere biopsy (SBB) involving the removal of a single blastomere from the eight-cell stage of a developing organism. ES cells may be characterized by the expression of markers such as, but not limited to SSEA-3, SSEA-4, TRA-1-60, TRA-1-81, and/or Alkaline phosphatase. Methods of generating and characterizing ES cells are known in the art and may be found in, for example, U.S. Pat. Nos. 7,029,913; 5,843,780; 6,200,806 (the contents of each of which are herein incorporated by reference in their entirety).

Induced pluripotent stem cells (iPSCs) may also be used to generate cells of the present disclosure. iPSCs may include cells with one or more properties such as, but not limited to (a) self-renewal (b) ability to differentiate to produce all types of cells in an organism and/or (c) be derived from a somatic cell. iPSCs may express markers such as, but not limited to SSEA3, SSEA4, SOX2, OCT3/4, Nanog, TRA160, TRA1818, TDGF1, Dnmt3b, FoxD3, GDF3, Cyp26a1, TERT, Zpf42. Methods of generating and characterizing iPS cells may be found in, for example, US Patent Publication Nos. US20090047263, US20090068742, US2009191159, US20090227032, US20090246875, and US20090304646 (the contents of each of which are herein incorporated by reference in their entirety). In some embodiments, the iPSCs may be derived from a T cell or non-T cell, a B cell, or any other cell from peripheral blood mononuclear cell, a hematopoietic progenitor cell, or any other somatic cell type.

In some embodiments, pluripotent stem cells may be derived from adult stem cells. Adult stem cells may be obtained from the inner ear, bone marrow, mesenchyme, skin, fat, liver, muscle, and/or blood of a subject such as subject. PSCs may also include embryonic stem cells derived from a placenta or umbilical cord; progenitor cells (e.g., progenitor cells derived from the inner ear, bone marrow, mesenchyme, skin, fat, liver, muscle, and/or blood).

### Effector Cells

An “effector cell” refers to any cell or cell type which, when in contact with or in proximity to a thymic cell, acquires the ability to execute, initiate or propagate a signal or a cell death trigger. “Contact or proximity” may refer to spatiotemporal closeness sufficient to enable cell-intrinsic or cell-extrinsic (e.g., cell-to-cell) signaling or other communication or interaction.

Effector cells described herein may be derived from pluripotent stem cells. In some embodiments, effector cells may be derived from embryonic stem cells, hematopoietic stem or progenitor cells, cells isolated from bone marrow, cord blood, peripheral blood, thymus, or the stem or progenitor cells may have been differentiated from embryonic stem cells (ESC) or induced pluripotent stem cells (iPSC) in vitro. Stem or progenitor cells from primary tissue or ESCs or iPSCs may be from human or non-human animals (e.g., mouse) in origin.

In some embodiments, the effector cell may be a hematopoietic cell. In some embodiments, the effector cell may be a lymphocyte. In some embodiments, the lymphocyte may be a CD45 positive lymphocyte.

Effector cells may be CD4+CD8− T cells, CD4− CD8+ T cells, CD34+CD7+ CDla+ cells, CD3+ TCRab+ cells, CD3+ TCRgd+ cells, CD3+ TCRab+CD4+CD8− cells, CD3+ TCRab+CD8+CD4− cells, CD3+ TCRab+CD4+CD8− CD45RO− CD45RA+ cells, CD3+ TCRab+CD8+CD4− CD45RO− CD45RA+ cells, CD3+ TCRab+CD4+CD8−CD45RO− 30 CD45RA+ CCR7+ cells, CD3+ TCRab+CD8+CD4− CD45RO− CD45RA+ CCR7+ cells, CD3+ TCRab+CD4+CD8− CD45RO− CD45RA+CD27+ cells, CD3+ TCRab+CD8+CD4− CD45ROCD45RA+ cells, CD27+, CD34+CD7+CD1a+ cells, CD34+CD5+CD7+ cells, CD34+CD5+CD7− cells, natural killer T cells, regulatory T cells, antigen-specific T cells, intraepithelial lymphocyte T cells, or cells that are CD45+, CDI 1b+, CDI lb−, CD15+, CD15−, CD24+, CD24−, CDI 14+, CD114−, CD182+, CD182−, CD4+, CD4−, CD14+, CD14−, CDlla+, CDlla−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD3 l+, CD3 l−, CD30+, CD30−, CD38+, and/or CD38− and/or cells that are positive for combinations thereof.

In some embodiments, the effector cell may be a T cell. T cells may be cultured T cells, e.g., primary T cells, or T cells from a cultured T cell line, e.g., Jurkat, SupTl, or T cells obtained from a mammal. If obtained from a mammal, the effector cells may be obtained from numerous sources, including, but not limited to, blood, bone marrow, lymph node, thymus, spleen, or other tissues or fluids. Effector cells may also be enriched for or purified. The T cells can be any type of T cells and can be of any developmental stage, including, but not limited to, CD4+/CD8+ double positive T cells, CD4+ helper T cells, e.g., Th1 and Th2 cells, CD4+ T cells, CD8+ T cells (e.g., cytotoxic T cells), peripheral blood mononuclear cells (PBMCs), peripheral blood leukocytes (PBLs), tumor infiltrating cells (TILs), memory T cells, naïve T cells.

In some embodiments, effector cells may be CCRXA−, CD3+, CD69−, MHC-1+, CD62L+, and/or CCR7+.

Effector cells may have a naïve T cell (TN) phenotype, central memory T cell (TcM) phenotype, or effector memory T cell (TEM) phenotype. The phenotypes of TN, TcM, and TEM cells are known in the art. For example, CCR7 and CD62L are expressed by TN and TcM cells but are not expressed by TEM cells. The transcription factors LEFI, FOXPI, and KLF7 are expressed by TN and TcM cells, but are not expressed by TEM cells. CD45RO and KLRGl are not expressed by TN cells, but are expressed by TEM cells (Gattinoni et al., Nat. Rev. Cancer, 12:671-84 (2012)). Alternatively, or additionally, TN and TcM cells may be characterized by longer telomeres as compared to those of TEM cells.

In some embodiments, effector cells may be TCRα+TCRβ+ cells. TCRα+TCRβ+ effector cells may be T cells expressing receptor expressing an alpha (α) chain and/or a beta (β) chain. TCR alpha and beta chains are known in the art.

Effector cell may be further modified. In further embodiments, the stem or progenitor cells may be genetically modified. For example, the stem or progenitor cells may express an exogenous T cell receptor (TCR) or a chimeric antigen receptor (CAR), or both. In further embodiments, the stem or progenitor cells may express an exogenous invariant natural killer T cell (INKT) associated TCR. In still further embodiment, the stem or progenitor cells express an exogenous antigen specific TCR or have an exogenous genetic modification of genes that modulate T cell differentiation, expansion, or function.

In some embodiments, effector cells may be FOXP3+ Tregs. Tregs can be generated via clonal diversion of mTECs, whereby expression of Aire in mTECs leads to expression of tissue-specific antigens which become surface displayed (i.e., on antigen presenting cells (APCs)). Autoreactive T cells that recognize the tissue-specific antigens give rise to FOXP3+ Tregs that can mediate peripheral tolerance (see Husebye, Eystein S., Mark S. Anderson, and Olle Kämpe. “Autoimmune polyendocrine syndromes.” New England Journal of Medicine 378.12 (2018): 1132-1141, incorporated herein by reference in its entirety)

### Supporting Cells

In some embodiments, cells of the present disclosure may include or may be cultured with supporting cells that aid in the generation of thymic cells and/or maintenance of thymic cells in culture. Non-limiting examples of supporting cells include hematopoietic non-T-cell progenitors, such as macrophages and dendritic cells (DCs); non-hematopoietic cells, such as epithelial cells and fibroblasts; stromal cells such as the progenitors of skeletal tissue, components such as bone, cartilage, the hematopoiesis-supporting stroma, and adipocytes. Supporting cells, in some embodiments, encourage the proliferation, survival, maturation, or function of thymic cells. In some embodiments, the supporting cells may be mesenchymal in origin.

Supporting cells may be non-immune cells that may be present in the thymic microenvironment. For example, the support cells may be fibroblasts, vascular smooth muscle cells (VSMCs), endothelial cells, and/or lymphatic endothelial cells. Support cells may also include Mesenchymal Stem Cells (MSCs) from different source including bone marrow derived MSCs, adipose tissue derived MSCs, thymus-derived MSCs, or iPS-derived MSCs (Tong Ming Liu et al Stem Cell Reports 2020 14:210; Samsonraj et al Stem Cells Translational Medicine 2017 6:2173); the contents of each of which are herein incorporated by reference in their entirety).

In some embodiments, the supporting cells may be neuroendocrine cells (expressing BEX1, NEUROD1), muscle-like myoid (expressing MYOD1, DES), and myelin positive epithelial cells (also herein myelin cells) (expressing SOX10, MPZ) described by Bautista et al. 2021 Nat Commun 12, 1096 (2021); the contents of which are herein incorporated by reference in its entirety. In some embodiments, mesenchymal cells may be associated with markers such as, but not limited to LAMA2, LAMA4, PDGFRA, PDGFRB, LUM, CSPG4, COLIA2, COL3A1, IGF1, FGF7, FGF10, FST, BMP4, SFRP2, WNT5A.

The mesenchymal cells may be positive or negative for one or more of these markers. In some embodiments, the mesenchymal cells may be positive for some marks described herein but may be negative for other markers.

In some embodiments, endothelial cells may be associated with one or markers such as, but not limited to, VEGFC, PECAM1, APLNR, PROX1, LYVE1, ACKR1, SELE, SELP, FN1, and/or TGFB1. The endothelial cells may be positive or negative for one or more of these markers. In some embodiments, the endothelial cells are adult vein endothelial cells, adult artery endothelial cells, embryonic stem cell-derived endothelial cells, iPS-derived endothelial cells, umbilical vein endothelial cells, umbilical artery endothelial cells, endothelial progenitors cells derived from bone marrow, endothelial progenitors cells derived from cord blood, endothelial progenitors cells derived from peripheral blood, endothelial progenitors cells derived from adipose tissues, endothelial cells derived from adult skin, or a combination thereof. In some embodiments, the umbilical vein endothelial cells are human umbilical vein endothelial cells (HUVEC).

In some embodiments, the supporting cells of the disclosure may be fibroblast and/or fibroblast-like cells. In some embodiments, the fibroblasts are human foreskin fibroblasts, human embryonic fibroblasts, mouse embryonic fibroblasts, skin fibroblasts cells, vascular fibroblast cells, myofibroblasts, smooth muscle cells, mesenchymal stem cells (MSCs)-derived fibroblast cells, or a combination thereof. In some embodiments the fibroblasts are normal human dermal fibroblasts (NHDFs).

In some embodiments, the supporting cells may be ciliated cells which are positive for ATOH1, GFI1, LHX3, and/or FOXJ1. In some embodiments, the supporting cells may be myelin cells that closely resemble Schwann cells that may be positive for SOX10, MPZ, MBP, and/or S100A1. In some embodiments, the supporting cells may be tuft cells which may be positive for markers GNB3, TRPM5, GNAT3, PLCB2, OVOL3, and/or POU2F3.

In some embodiments, the supporting cells may be ionocyte population cells which may be positive for FOXI1, ASCL3, CFTR, and/or CLCNKB.

Other non-limiting examples of supporting cells include, hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells.

### Thymic Organoids

In some embodiments, the compositions of the disclosure may include a thymic organoid. An organoid is an in vitro, three-dimensional, miniature recapitulation of an organ. A thymic organoid may be an in vitro three-dimensional, miniature version of the thymic organ that may mimic the physiology and function of a human thymus. Methods of preparing thymic organoids are described in the International Patent Publication WO2019060336, the contents of which are herein incorporated by reference in its entirety.

In some embodiments, effector cells may be prepared by differentiating the pluripotent stem cells or progenitor cells into lymphocytes by culturing PSC or progenitor cells with thymic cells. In some embodiments, the thymic cells may express a Notch ligand. In some embodiments, the Notch ligand may be Delta-like 1 (DLL1). In some embodiments, the Notch ligand is Delta-like 4 (DLL4). In some embodiments, the Notch ligand is one described herein or in the art, such as in U.S. Pat. No. 7,795,404, which is herein incorporated by reference in its entirety. Effector cells of the present disclosure may be prepared using the thymic organoid cell culture systems. In some embodiments, the method further comprises contacting the co-cultured stem or progenitor cells and stromal cells with Fit-3 ligand and/or IL-7 and/or Stem Cell Factor/Kit ligand and/or thrombopoietin. In some embodiments, differentiating the stem or progenitor cell into a T cell comprises: culturing a three dimensional (3D) cell aggregate, comprising: a) a selected population of supporting cells that endogenously or exogenously express a Notch ligand; b) a selected population of stem or progenitor cells; with a serum-free medium comprising B-27® supplement, xeno-free B-27® supplement, GS2 l ™ supplement, ascorbic acid, Flt-3 ligand, IL-7, or a combination thereof. Any of the methods for generating lymphocytes from stem cells or progenitor cells described in International Patent Publication WO2017075389 may be useful in the present disclosure (the contents of which are herein incorporated by reference in their entirety).

In some embodiments, the thymic organoids may be based on the artificial thymic organoids described by Seet C S, et al. Nat Methods. 2017; 14(5):521-530 (the contents of which are herein incorporated by reference in its entirety). To prepare thymic organoids, thymic cells may be harvested by trypsinization and resuspended in serum free culture medium (“RB27”) which may include RPMI 1640 (Corning, Manassas, VA), 4% B27 supplement (ThermoFisher Scientific, Grand Island, NY), 30 μM L-ascorbic acid 2-phosphate sesquimagnesium salt hydrate (Sigma-Aldrich, St. Louis, MO) reconstituted in PBS, 1% penicillin/streptomycin (Gemini Bio-Products, West Sacramento, CA), 1% Glutamax (ThermoFisher Scientific, Grand Island, NY), 5 ng/ml rhFLT3L and 5 ng/ml rhIL-7 (Peprotech, Rocky Hill, NJ). Different ratios of thymic cells and effector cells may be prepared in 1.5 ml Eppendorf tubes and centrifuged at 300 g for 5 min. at 4° C. in a swinging bucket centrifuge. Supernatants were carefully removed, and the cell pellet was resuspended by brief vortexing. For each organoid, a 0.4 μm Millicell trans well insert (EMD Millipore, Billerica, MA; Cat. PICM0RG50) may be placed in a 6-well plate containing 1 ml RB27 per well. To plate the organoids, inserts were taken out and rested on the edge of plate to drain excess medium. The cell slurry may be adjusted to 5 μl per organoid, drawn up in with a 20 μl pipet tip and plated by forming a drop at the end of the pipet tip which was gently deposited onto the cell insert. The cell insert may be placed back in the well containing 1 mL. RB27. Medium may be changed completely every 3-4 days by aspiration from around the cell insert followed by replacement with 1 ml with fresh RB27/cytokines. In some embodiments, organoids may be cultured in this manner for up to 10 weeks, 15 weeks, 20 weeks, 25 weeks, or 30 weeks.

At the indicated times, organoids cells were harvested by adding FACS buffer (PBS/0.5% bovine serum album/2 mM EDTA) to each well and briefly disaggregating the organoids by pipetting with a 1 ml “P1000” pipet, followed by passage through a 50 μm nylon strainer. In some experiments, single cell suspensions of MS5-hDLL1 cells were γ-irradiated at the indicated doses prior to use in organoids.

Thymic organoid effector cell co-cultures may be prepared as described in Seet C S, et al. Nat Methods. 2017; 14(5):521-530 (the contents of which are herein incorporated by reference in its entirety). Thymic cells may be seeded into 0.1% gelatin-coated 12 well plates 1-2 days prior to use to achieve 70-80% confluence. Medium may be aspirated from monolayers and 1.5×10⁴ FACS purified effector cells (CD34+CD3-hematopoietic cells) may be plated with thymic organoids in 2 ml of medium composed of MEMα, 20% FBS, 30 UM L-Ascorbic acid, 5 ng/ml rhFLT3L, and 5 ng/ml rhIL-7. In some embodiments, effector cells may be transferred to thymic organoids every 4-5 days by harvesting cells, filtering through a 50 μm nylon strainer, and replating in fresh medium. When confluent, cells were split into multiple wells containing fresh stromal layers.

In some embodiments, thymic cells of the disclosure may be combined with double negative day 14 T cells to form a cluster of cells which may then be deposited onto the trans well as an “organoid” and maintained in an air-liquid interface culture condition. In some embodiments, cells can be harvested from the medium every few days to assess T cell maturation.

### Thymic Support System (TSS)

In some embodiments, the compositions of the disclosure may include a thymic support system. As used herein, a thymic support system refers to a system of one or more non-living components that may interact with the cells of the disclosure to provide structure, support, and/or essential cues for the viability, function, proliferation and/or differentiation of the cells of the disclosure. In one embodiment, the TSS promotes differentiation of iPS cells, AFE cells, VPE cells, DE cells and/or TEP cells. In some embodiments, TSSs may mimic thymus or thymic tissue structure in a three-dimensional manner.

The thymic support system may include one or more components such as, polymers, matrix, and matrix components and optionally one or more agents.

In some embodiments, TSSs may (i) provide structure for adhesion, proliferation, and differentiation of thymic cells, (ii) create a suitable biomechanical environment, and/or (iii) permit the dissemination of nutrients and oxygen.

In some embodiments, TSSs may mimic thymus tensegrity, viscoelasticity and/or stiffness (Engler A. J., et al. Cell. 2006; 126(4):677-89; the contents of which are herein incorporated by reference in its entirety).

In some embodiments, TSSs may have an architecture that allows for T cell migration towards and/or away from the TSSs, and/or cells of the disclosure.

In some embodiments, the TSSs may be “bioinert,” “biocompatible,” “bioactive” or “resorbable,” depending on their biological response in vivo. As used herein, the term “biocompatible,” as used herein, refers to a substance or object that performs its desired function when introduced into an organism without inducing an inflammatory response, immunogenicity, or cytotoxicity to native cells, tissues, or organs, or to cells, tissues, or organs introduced with the substance or object.

In some embodiments, TSSs may include a porous network through which oxygen, nutrients and metabolites can be exchanged. Regarding porosity, TSSs may also be biphasic including a region of the TSS with a highly porous morphology to mimic thymic medulla and another region of the TSS with less porous morphology to mimic thymic cortex morphology.

Other desirable properties of TSSs include, but are not limited to, capacity for high number cell delivery to desirable sites, injectable into a subject without disruption of its properties, (e.g., pre-formed cell structure), reduced ethical concerns and/or reduced batch-to-batch variability.

In some embodiments, TSSs may include decellularized thymic tissue. This approach provides an opportunity to grow thymic cells in their native environment while retaining biomechanical and/or biochemical cues. Decellularization may be achieved by any methods known in the art, such as by whole organ perfusion followed by detergent wash. Decellularized thymic tissue may also be homogenized and cross-linked to produce TSSs of the disclosure. In some embodiments, compositions of the disclosure may include decellularized extracellular matrix extracted from thymic tissues. The thymic tissues may be fetal, neonatal, juvenile, or adult. The thymic tissues may be autologous, allogenic, or xenogeneic.

Cells may be patterned within or onto the TSSs of the disclosure by selective polymerization of the polymers of the disclosure or by patterning of the cells using an electrical field or both. The cells may be patterned by locating the cells within specific regions of relatively homogeneous preparations of polymers (resolution up to about 5 microns) or by creating patterned polymer scaffolds of defined patterns wherein the living cells are contained within (resolution up to about 100 microns). Patterning may be performed without direct, mechanical manipulation or physical contact and without relying on active cellular processes such as adhesion of the cells.

The methods described herein can be used for the production of any of a number of patterns in single or multiple layers including geometric shapes or a repeating series of dots with the features in various sizes. Alternatively, multilayer biopolymer gels can be generated using a single mask turned in various orientations. The formation of high resolution patterned cells in 3-dimensions can be achieved by methods other than photopolymerization, such that the limitations of the method are overcome.

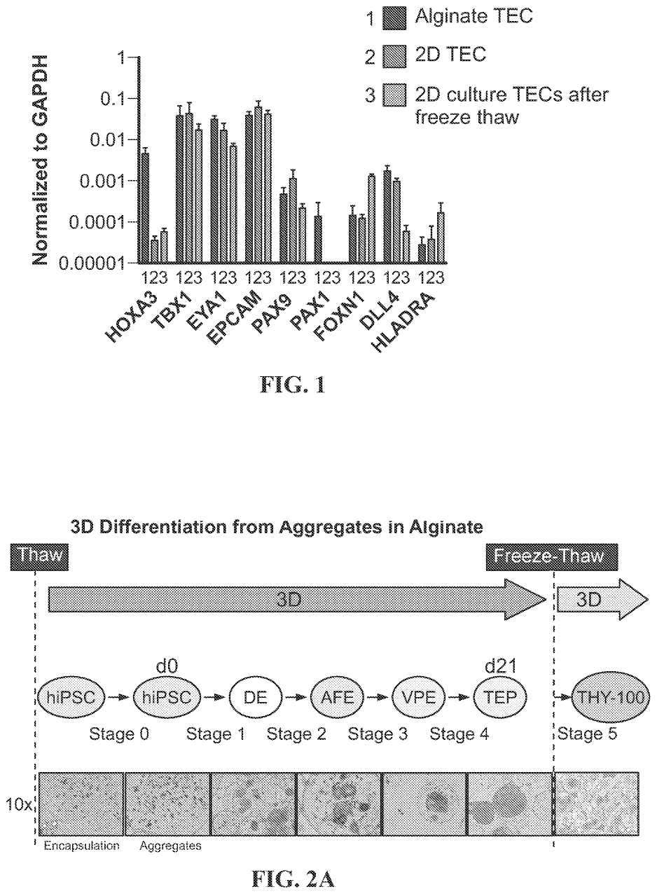

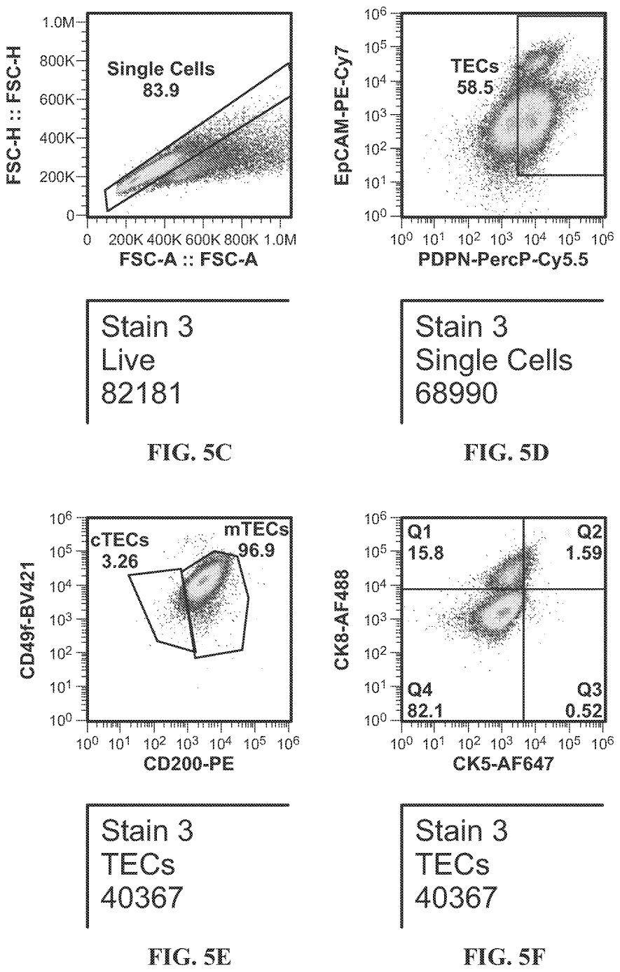

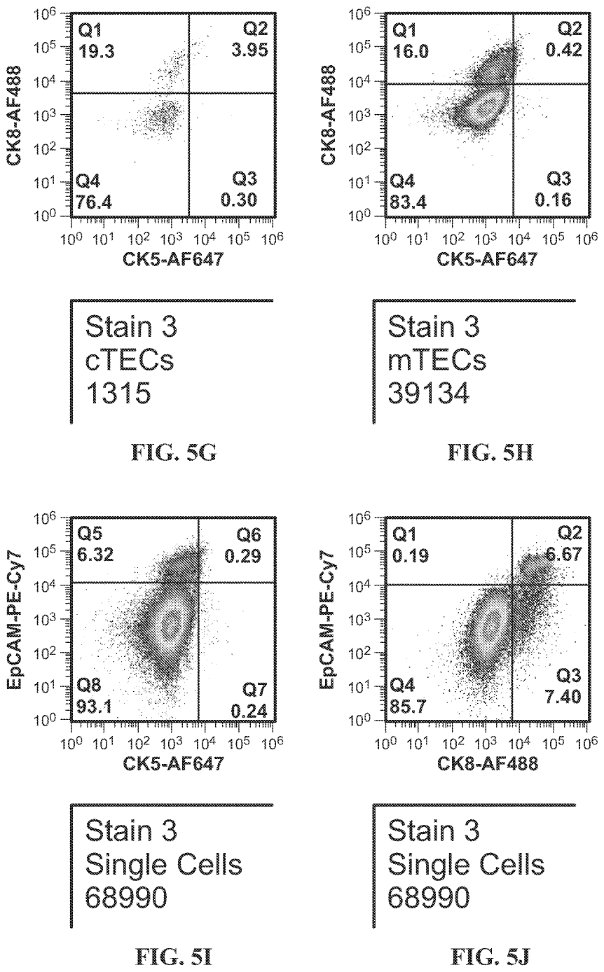

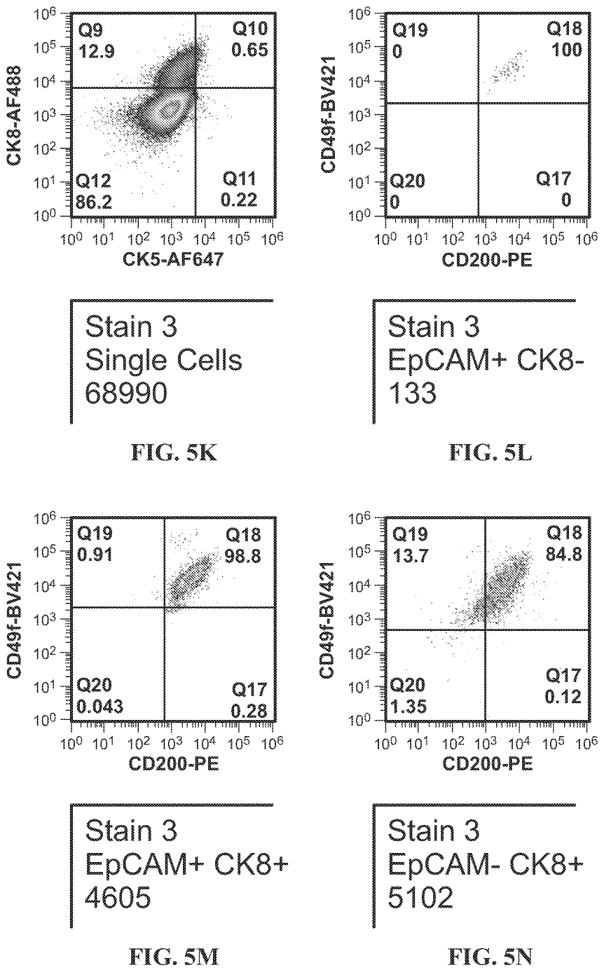

TSSs of the disclosure may be organized into structures that resemble cylinders, rods, strings, filaments, or networks. Such architecture can lead to enhanced integration of the compositions of the disclosure into host organisms as evidenced by improved blood supply, and/or improved vasculature. The TSSs may be patterned to allow for the organization of the cells into clusters or islands. Patterning may be performed according to the process of FIG. 1A or FIG. 5A of US Patent Publication US20210213171, the contents of which are herein incorporated by reference in their entirety. TSSs may also be fabricated using custom 3D printer technology.

### Polymers

In some embodiments, TSSs may include a polymer. Any substance or a blend of the natural or synthetic source may be used in thymic support systems. In some embodiments, TSSs may be a biogenic polymer, a synthetic polymer, or a composite thereof.

In some embodiments, the polymer may be a hydrophilic polymer. Hydrophilic polymers contain polar or charged functional groups, rendering them soluble in water. They can interact with or be dissolved by water or other polar substances. In some embodiments, the one or more polymers (or monomers thereof) or at least one of the one or more polymers has one or more hydrophilic groups. In some embodiments, each of the one or more hydrophilic groups may be individually selected from the group of: —NH2, —COOH, —OH, —CONH2, —CONH−, and —SO3H. Each of the one or more polymers can be individually selected from a biogenic polymer and a synthetic polymer. In some embodiments, all the polymers in the hydrogel are biogenic polymers. In some embodiments, all the polymers in the hydrogel are synthetic polymers. In some embodiments, where two or more polymers are present, at least one polymer is a biogenic polymer and at least one polymer is a synthetic polymer.

In some embodiments, the TSSs may be temperature-responsive polymers (e.g., pNIPAM, PVME) that transition between hydrophobic and hydrophilic states at certain temperatures, allowing the control of cell culture and growth and subsequently the deposition of ECM and the formation of cell sheets that adhere to biological surfaces.

The molecular weight of the polymers can be any suitable molecular weight. In some embodiments, the polymers present in the hydrogel each has an average molecular weight that is independently selected from about 100 to about 1000 Da, about 100 to about 900 Da, about 100 to about 800 Da, about 100 Da to about 700 Da, about 200 Da to 600 Da, about 300 Da to about 600 Da, about 400 Da to about 600 Da, about 500 Da to about 600 Da, about 525 Da to about 600 Da, about 550 Da to about 600 Da, 575 Da to about 600 Da.

In one embodiment, the cells of the disclosure such as, but not limited to iPS cells, AFE cells, VPE cells, DE cells, TEP cells, and/or TECs are encapsulated in the polymers described herein. The 3-D configuration of TECs in the thymic microenvironment is important to maintain their thymic epithelial gene signature. Thymic cells prepared as aggregates in biocompatible hydrogel can maintain their molecular properties and prolong their survival for up to 7 days in vitro. Encapsulating the cells of the disclosure is also advantageous in scaling up the manufacturing of thymic cells for clinical applications. In one embodiment, the polymers of the disclosure provide critical 3-D matrix support for the survival of iPSC-derived thymic cells.

In some embodiments, the concentration of the polymer is about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, about 50%, about 55%, about 60%, about 65%, about 70%, about 75%, about 80%, or more, about 0.1% to 0.5%, about 0.5% to 1%, about 1% to 1.5%, about 1.5% to 2%, about 2% to 2.5%, about 2.5% to 3%, about 3% to 3.5%, about 3.5% to 4%, about 4% to 4.5%, about 4.5% to 5%, about 5% to 5.5%, about 5.5% to 6%, about 6% to 6.5%, about 6.5% to 7%, about 7% to 7.5%, about 7.5% to 8%, about 8% to 8.5%, about 8.5% to 9%, about 9% to 9.5%, about 9.5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 70% to 75%, about 75% to 80%, or more.

### Biogenic Polymers

Any polymer derived from a natural source, or an organism may be defined as a biogenic polymer and may be used in the thymic support systems.

Biogenic polymers may include, but are not limited to, one or more of a protein, a polypeptide, a polysaccharide, a lipid, a nucleic acid, and a glycosaminoglycan. Biogenic polymers may include, but are not limited to, one or more of silk, fibroin, sericin, keratin, alpha-keratin, beta-keratin, alginate, elastin, fibrillin, fibrillin-1, fibrillin-2, fibrillin-3, fibrillin-4, fibrinogen, fibrin, fibronectin, laminin, collagen, collagen I, collagen II, collagen III, collagen IV, collagen V, collagen VI, vimentin, neurofilament, light chain neurofilament (NF-L), medium chain neurofilament (NF-M), heavy chain neurofilament (NF-H), amyloid, alpha-amyloid, beta-amyloid, actin, myosin, titin, gelatin, chitin, hyaluronic acid, D-glucuronic acid, legumine, viciline, and/or D-N-acetylglucosamine.

Based on their monomeric units and structure, biogenic polymers may be categorized as polypeptide-based biogenic polymers, polysaccharide-based biogenic polymers, and polynucleotide-based biogenic polymers.

In some embodiments, the biogenic polymer may be a polypeptide-based biogenic material. Non-limiting examples of polypeptide-based biogenic polymers include, collagen, fibrin, fibrinogen, gelatin, silk, elastin, myosin, keratin, and/or actin. In some embodiments, the biogenic polymer may be collagen, which is a primary structural element of the ECM and has several functional characteristics that help to bind the cell, proliferation, differentiation, and secretion of the ECM. In some embodiments, collagen may consist of three identical chains (homotrimers) e.g., collagen type II, III, VII, VIII, and/or X. Collagen may also include two or more different chains (heterotrimers) e.g., in collagen types I, IV, V, VI, IX, and XI. Collagen composition may be modified to achieve improved biological activity and mechanical properties of the final scaffold by combining with other molecules such as hyaluronic acid (HA), chitosan, and chondroitin sulphate (CS). In some embodiments, the biogenic polymer may be gelatin, which is the result of degradation derived from insoluble collagen by disintegration or denaturation. In some embodiments, the biogenic polymer may be silk, a natural protein-based polymer derived from various Lepidoptera larvae, such as spiders, as well as silkworms.

In some embodiments, the biogenic polymer may be a polysaccharide-based biogenic polymer. Non-limiting examples of polysaccharide-based biogenic polymers include, chitin, chitosan, alginate, hyaluronic acid, cellulose, agarose, dextran, and/or glycosaminoglycans, polysaccharide-based biogenic polymer may be made of different units of monosaccharide or disaccharide chains (e.g., starch, cellulose). The effect is an incredibly large number of structurally diverse polysaccharides as numerous distinct saccharide isomers are mixed by utilizing a range of chemical bonds. In some embodiments, the biogenic polymer may be a based on a structural polysaccharide e.g., cellulose in plants and chitin in crustacean shells. Chitin is generally found in shells of crustaceans and its derivative chitosan is obtained by deacetylation of chitin. These are glycosaminoglycan like natural cationic polysaccharides. In one embodiment, the polymer may be sulfated dextran.

In some aspects, the biogenic polymer may be starch and glycogen.

In some embodiments, the biogenic polymer may be hyaluronic acid (HA), a linear polysaccharide, ubiquitous and extremely biologically compatible in the ECM of mammals. HA includes functional groups such as carboxylic acids and alcohols that can be used for the implementation of functional domains or the development of a hydrogel by connecting them.

In some embodiments, the biogenic polymer may be a polynucleotide-based biogenic polymer. Non-limiting examples of polynucleotide-based biogenic polymers include, DNA, linear plasmid DNA, and/or RNA.

In some embodiments, the polymers of the disclosure may be a combination of two biogenic polymers e.g., heparin and dextran as described in International Patent Publication WO2022015902, the contents of which are herein incorporated by reference in their entirety.

### Synthetic Polymers

In some embodiments, the TSS may include a synthetic polymer. As used herein, the term “synthetic” means produced, prepared, and/or manufactured by the hand of man. Synthesis of polymers or other molecules of the present disclosure may include chemical or enzymatic synthetic methods.

In some embodiments, the synthetic polymer may be Poly(α-hydroxy esters) including PCL, PGA, PLA, and their copolymer PLGA and poly(ethers) including PEO and PEG, PVA, and PU.

In some embodiments, the synthetic polymer may be poly lactic acid (PLA), a gradually crystallizing semicrystalline polymer. PLA may be prepared from the lactic acid (LA) monomer through the fermentation process of natural resources such as wheat and grain or by various routes of polymerization as a petrochemical derivative. In some embodiments, PLA may be poly(L-lactic acid) (PLLA), poly(D,L-lactic acid) (PDLA), and/or poly(D,L-lactic acid) (PDLLA). In some embodiments, the synthetic polymer may be polyglycolic acid (PGA).

In some embodiments, the synthetic polymer may be polylactic-co-glycolic acid (PLGA), a random ring-opening copolymer of PLA and PGA. In some embodiments, the synthetic polymer may be Polycaprolactone (PCL), a semicrystalline and aliphatic polymer. In some embodiments, the synthetic polymer may be poly(ethylene oxide) (PEO), a hydrophilic polymer that is inert with minimal antigenicity, immunogenicity, cell adhesion, and protein binding. In some embodiments, the synthetic polymer may be polyurethane (PU) which contains a urethane moiety in its repeating units. The reaction of diisocyanate with polyol normally produces these polymers.

Synthetic polymers may include, but are not limited to, one or more of a poly(urethane), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), a PLA, a PGA, a PLGA, a polyhydroxybutyrate (PHB), Polypropylene fumarate (PPF), a polyvinyl alcohol (PVA), a polypropylene carbonate, a polyanhydride, a polyphosphazene, a polygermane, a polyorthoester, a polyester, a polyamide, a polyolefin, a polycarbonate, a polyaramide, a polyimide, a PCL, and a copolymer, derivative, or combination thereof.

In some embodiments, the one or more polymers are each individually selected from polyethylene glycol (PEG), chitosan, Poly(2-hydroxyethyl methacrylate) (PHEMA), 2-Hydroxyethyl methacrylate (HEMA), Hydroxyethoxyethyl metha-crylate (HEEMA), Hydroxy diethoxyethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic polyallylammonium chloride, Poly(N-isopropyl acrylamide) (PNIPAM), self-assembling peptides, acrylate-modified PEG and acrylate-modified hyaluronic acid, heparin, amine end-functionalized 4-arm star-PEG, or any combination thereof. In some embodiments, at least one of the one or more polymers is PEGDA. In some embodiments, one or more of the polymers is PEGDA.

In some embodiments, the present disclosure provides a synthetic polymer comprising a polysaccharide that has been modified by converting one or more groups present in the polysaccharide into negatively charged functional groups, wherein the negatively charged groups provide an amount of negative charge to the synthetic polymer sufficient to promote one or more of binding of growth factors, growth factor activity and vascularization. In some embodiments, the functional groups in the polysaccharide that are converted into the negatively charged groups may be hydroxyl groups. In some embodiments, the polysaccharide of the synthetic polymer may be dextran, alginate, agarose, chondroitin sulfate, chitin/chitosan, cellulose, starch, hyaluronic acid, galactogen, inulin, pectin, or glycogen. As a non-limiting example, the synthetic polymer may be a heparin mimetic described in International Patent Publication WO2022015902, the contents of which are herein incorporated by reference in their entirety.

### Composite Polymers

Biogenic and synthetic polymers may be combined to produce composite polymers that combine the features of both polymer types to produce composite polymers that have improved features. For example, thymic cells of the disclosure may be cultured with the composite polymer of collagen-PCL (see D. J. Choi, et al. J. Biotechnol. 2015, 205, 47; the contents of which are herein incorporated by reference in its entirety). Thymic cells and/or thymic cell organoids may also be prepared using composite polymers of gelatin and PEG (see A. B. Suraiya, ACS Biomater. Sci. Eng. 2020, 6, 2198; the contents of which are herein incorporated by reference in its entirety).

### Hydrogels

In some embodiments, the polymer may be a hydrogel. Hydrogels are generally prepared by translating hydrophilic polymers solution into 3D network structure via physical or chemical crosslinking. During this process, hydrogels can encapsulate cells homogeneously and provide cells a 3D microenvironment similar to the native extracellular matrix (ECM). Cell behaviors and functions in vivo are affected by the stimuli that are produced by the surrounding ECM. Similarly, the structures and physiochemical properties of hydrogels provide critical cues to control the functions of the embedded cells. The structures and physiochemical properties of hydrogels may be designed and controlled through selecting different polymers, crosslinking methods, and fabrication strategies.

Hydrogels may be prepared from biogenic polymers such as, but not limited to, polypeptide-based polymers (such as gelatin, collagen, fibrin, and silk fibroin) and polysaccharide-based biogenic polymers (such as hyaluronic acid (HA), chondroitin sulfate (CS), alginate, chitosan). Collagen, as the main ECM component of various tissue, may be used for hydrogel preparation. In some embodiments, collagen derivative, gelatin, which has a higher solubility, may also be used to prepare hydrogels in the present disclosure. Hyaluronic acid (HA), a glycosaminoglycan is commonly prevalent in body fluids and ECM may be used for hydrogel preparation. Other polysaccharide-based biogenic polymers, such as alginate (obtained from bacteria and brown seaweed) and chitosan (derived from chitin that is produced from the shells of crabs and shrimps), may also be used in the present disclosure.

Hydrogels of the present disclosure may also be prepared using synthetic polymers such as, but not limited to, poly(ethylene glycol) (PEG), poly(vinyl alcohol) (PVA), poly(N-isopropylacrylamide) (PNIPAM), and polyacrylamide (PAM) (Darnell et al., 2013).

Hydrogels as described herein may include EAK16-II/EAKIIH6 self-assembling hydrogels as described by A. Tajima et al. Clin. Immunol. 2015, 160, 82; the contents of which are herein incorporated by reference in their entirety.

In some embodiments, hydrogels may be prepared from polymers of hydrophilic monomers. As used herein, a hydrophilic monomer refers to any monomer which, when polymerized, yields a hydrophilic polymer capable of forming a hydrogel when contacted with an aqueous medium such as water. Examples of hydrophilic monomers include, but are not limited to, hydroxyl-containing monomers such as 2-hydroxyethyl methacrylate, 2-hydroxyethyl acrylate, 2-hydroxyethyl methacrylamide, 2-hydroxyethyl acrylamide, N-2-hydroxyethyl vinyl carbamate, 2-hydroxyethyl vinyl carbonate, 2-hydroxypropyl methacrylate, hydroxyhexyl methacrylate and hydroxyoctyl methacrylate; carboxyl-containing monomers such as acrylic acid, methacrylic acid, itaconic acid, fumaric acid, crotonic acid, maleic acid and salts thereof, esters containing free carboxyl groups of unsaturated polycarboxylic acids, such as monomethyl maleate ester, monoethyl maleate ester, monomethyl fumarate ester, monoethyl fumarate ester and salts thereof; amide containing monomers such as (meth)acrylamide, crotonic amide, cinnamic amide, maleic diamide and fumaric diamide; thiol-containing monomers such as methanethiole, ethanethiol, 1-propanethiol, butanethiol, tert-butyl mercaptan, and pentanethiols; sulfonic acid-containing monomers such as p-styrenesulfonic acid, vinylsulfonic acid, p-a-methylstyrene sulfonic acid, isoprene sulfonide and salts thereof.

In some embodiments, a hydrogel may be formed by self-assembly of one or more types of hydrophilic polymers in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains may aggregate or coagulate in an aqueous medium, which may form a three-dimensional network, thereby entrapping molecules of water to form a hydrogel.

Composites of biogenic polymers and synthetic polymers may also be used. For example, PAM, PVA, PNIPAM, and PEG hydrogels may be blended with gelatin. The cell spreading, and proliferation in these hybrid hydrogels may be enhanced compared with the cells 3D cultured in synthetic polymer-based hydrogels.

### Cross-Linked Polymers

Cross-linking, as used herein may be defined as the induction of chemical or physical links among polymer chains. Polymers of the disclosure may be cross-linked to modify mechanical, biological and/or degradation properties of the polymers of the disclosure.

In some embodiments, hydrophilic polymers may be cross-linked to form hydrogels.

Various methods for cross-linking of polymers chains are known in the art and may be used herein. The methods may be selected depending on the materials chemistry and expected functions. In some embodiments, cross-links may be formed by covalent bonds or ionic bond.

Polymers may be physically crosslinked under very mild conditions without the utilization of crosslinking agents that often cause toxicity to cells or may affect the activity of biological molecules Physical cross-linking methods include, but are not limited to ionic interaction, guest-host interaction, and thermo-gelation.

In some embodiments, polymers described herein may be chemically cross-linked. In some embodiments, some or all of the crosslinking are formed via a Thiol-Michael addition reaction, such as a Thiol-Michael addition click reaction. Chemically cross-linked hydrogels may have stronger binding energy and improved flexibility due to the nature of the cross-linking reactions. Hydrophilic polymers have many functional groups, such as OH, COOH, and NH2. 3D network can be established by covalent bonding between these functional groups using glutaraldehyde and EDC/NHS. In some embodiments, chemical cross-linking may be achieved by photopolymerization (using photoreactive moieties such as methacrylate or acrylate groups), enzyme-enabled crosslinking (using enzymes like transglutaminase and horse radish peroxidase), click chemistry, and/or using Schiff base reaction (via the coupling between aldehyde and amine groups in polymer chains).

Examples of chemical compounds that act as cross-linking agent include, but are not limited to, dextran dialdehyde, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisaciylamide, N,N′-methylenebis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, or mixtures thereof.

In some embodiments, cross-links may be degradable or non-degradable cross-links.

In some embodiments, the cross-linkers may be a peptide cross-linker. As a non-limiting example, the cross-linker may be a protease degradable cross-linker e.g., VPM peptide GCRDVPMSMRGGDRCG (SEQ ID NO: 1) that is rapidly cleaved by matrix metalloproteinase (MMP)-1 and MMP-2 proteases. In some embodiments, the cross-linker may be a matrix metalloproteinase (MMP)-cleavable peptide. In some embodiments, said peptide comprises an amino acid sequence CGPQGIAGQGCR (SEQ ID NO: 2), GPQGIAGQ (SEQ ID NO: 3), GPQGIWGQ (SEQ ID NO: 4), VPMSMRGG (SEQ ID NO: 5), QPQGLAK (SEQ ID NO: 6), GPLGLSLGK (SEQ ID NO: 7), or GPLGMHGK (SEQ ID NO: 8). Additional MMP-cleavable peptides may be found in Tu, Y. et al. Smart Pharmaceutical Nanocarriers; 83-116 (2016), the entire contents of which are incorporated by reference herein.

In some embodiments, the polymers of the disclosure may be cross-linked PEG polymers. As a non-limiting example, PEG polymers may be cross-linked with a maleimide functional group. In one embodiment, the cross-linked PEG polymer may be a 4-arm PEG-maleimide (PEG-4MAL).

### Extracellular Matrix Components (ECMC)

The compositions of the disclosure may include one or more components of the extracellular matrix. In some embodiments, ECMC may be tethered, or decorated onto polymers of the disclosure for presentation to the cells of the disclosure.

In some embodiments, ECMC may be extracellular matrix proteins. In some aspects, the ECMC may be a biogenic polymer. Non-limiting examples of extracellular matrix proteins include, but are not limited to, fibronectin, laminin, vitronectin, tenascin, entactin, thrombospondin, elastin, gelatin, collagen, fibrin, merosin, ankaline, chondronectin, link protein, bone sialo acid protein, osteocalcin, osteopontin, epinectin, byaluronectin, unjulin, Epiligrin and carinine.

Extracellular matrix proteins useful in the present disclosure may be biogenic or purified from human or animal tissue. Alternatively, the ECM protein may be a genetically engineered recombinant protein or an original synthetic product. The ECM protein may be a whole protein or a peptide fragment. Examples of ECM protein that may be useful include laminin, type I collagen, type IV collagen, fibronectin and vitronectin.

In some embodiments, the extracellular matrix component may be Matrigel (or similar commercially available products such as Geltrex), a laminin-111-rich basement membrane extracted from Engelbreth-Holm-Swarm mouse sarcoma. In some embodiments, ECMC may be generated from naturally occurring materials, such as fibrin, collagen, or hyaluronic acid, or from synthetic hydrogel.

In some embodiments, ECMCs may include peptides that match short key amino acid sequences of ECM proteins. ECMCs may include peptides that binding to integrin receptor subunits α5, α6, αv, β1 and β5. In some embodiments, ECMCs may be fibronectin-derived three-amino-acid peptide Arg-Gly-Asp (RGD), which binds to both αvβ3 and αvβ5 integrins. A cyclic form of RGD was identified as the most effective peptide for hPSC culture; the cyclic RGD peptide compared favorably with other peptides derived from laminin, fibronectin and vitronectin and may be used in the present disclosure (Lambsbead J W et al. Sci. Rep 8, 701 (2018); the contents of which are herein incorporated by reference in its entirety). In some embodiments, the peptide may be CGRGDS (SEQ ID NO: 9).

Other examples of ECMCs include peptides corresponding to the collagen motif GFXGER (SEQ ID NO: 10), the laminin motif IKVAV (SEQ ID NO: 11) and YIGSR (SEQ ID NO: 12), MNYYSNS (SEQ ID NO: 13) or CNYYSNS (SEQ ID NO: 14), DAPS (SEQ ID NO: 15), AELDVP (SEQ ID NO: 16), VALDEP (SEQ ID NO: 17), NGRAHA (SEQ ID NO: 18), peptides derived from vitronectin, and/or bone sialoprotein.

In some embodiment, the ECMC may be a synthetic matrix component.

In some embodiments, ECMC may include growth factors, such as transforming growth factor (TGF) family peptides (for example, TGF-β), fibroblast growth factors (FGFs), integrins, as well as enzymes, such as matrix metalloproteinases (MMPs).

### Agents

TSSs may be prepared with one or more agents. The one or more agents can be released (either continuously or controlled), from the TSSs to be available for interactions with cells of the disclosure. Agents may be biological agents or chemical agents. The agents may promote survival, growth, expansion, differentiation, and/or one or more functions of the cells of the disclosure.

In some embodiments, an agent may be a biological agent. As used herein, “biological agent” refers to any compound, composition, biopolymer, molecule and the like that is made by a living organism and include, without limitation, polynucleotides (e.g., DNA, RNA), peptides and polypeptides, and chemical compounds.

In one embodiment, the biological agent may be a protein tag e.g., a biotin molecule.

In some embodiments, the biological agent may be an antibody or fragment thereof. As a non-limiting example, the antibody may be an EpCAM antibody that binds with polymers of the disclosure via an adaptor complex. Such antibodies may allow for sequester thymic cells of the disclosure within, or on the TSSs of the disclosure (see A. Tajima, et al. Fan, Clin. Immunol. 2015, 160, 82; the contents of which are herein incorporated by reference in its entirety).

The one or more agents can each individually be selected from the peptides, polypeptides, antibodies, aptamers, ribozymes, guide sequences for ribozymes that inhibit translation or transcription of essential proteins and genes, hormones, immunomodulators, antipyretics, anxiolytics, antipsychotics, analgesics, antispasmodics, anti-inflammatories, anti-histamines, anti-infectives, radiation sensitizers, agent sensitizers, imaging agents, chemotherapeutic agents, chemokines, cytokines, anti-migratory compounds capable of inhibiting chemokine receptors to decrease cell invasion, or any combination thereof.

In some embodiments, the biological agent may be a ligand, which may interact with a counterpart receptor that is specific to the ligand and together transmit a message or signal to take on a particular function or phenotype. For example, cells as described herein can have an exogenous nucleotide sequence encoding a ligand that may be introduced (or have been previously introduced) into the cells by transfection or transduction. As a non-limiting example, the ligand may be a Notch ligand. The term “Notch ligand” as used herein includes intact (full-length), partial (a truncated form), or modified (comprising one or more mutations, such as conservative mutations) notch ligands as well as Notch ligands from any species or fragments thereof that retain at least one activity or function of a full-length Notch ligand. Also included are peptides that mimic notch ligands. Notch ligands may be “canonical notch ligands” or “noncanonical notch ligands.” Canonical notch ligands are characterized by extracellular domains typically comprising an N-terminal (NT) domain followed by a Delta/Serrate/LAG-2 (DSL) domain and multiple tandemly arranged Epidermal Growth Factor (EGF)-like repeats. The DSL domain together with the flanking NT domain and the first two EGF repeats containing the Delta and OSM-11-like proteins (DOS) motif are typically required for canonical ligands to bind Notch. The intracellular domains of some canonical ligands contain a carboxyterminal PSD-95/Dlg/ZO-l-ligand (PDZL) motif that plays a role independent of Notch signaling. C. elegans DSL ligands lack a DOS motif but have been proposed to cooperate with DOS-only containing ligands to activate Notch signaling. Illustrative canonical notch ligands include, but are not limited to, Delta-like ligand 4 (DLL4), Delta-like ligand 1 (DLLl), Jagged 1 (JAG1), Jagged 2 (JAG2), Delta-like ligand 3 (DLL3), and X-delta 2; other similar illustrative canonical ligands are contemplated in additional embodiments. Non-canonical notch ligands lack a DSL domain (Delta/Serrate/LAG-2), are structurally diverse, and include integral- and GPI-linked membrane proteins as well as various secreted proteins. Where a “notch ligand fragment” or a “canonical notch ligand fragment” is referenced herein, it is contemplated that the fragment is a fragment that binds notch. Examples of non-canonical notch ligands include, but are not limited to, Contactin-1, NOV/CCN3, Contactin-6, Periostin/OSF-2, DLK2/EGFL9, Pref-1/DLKl/FAl, DNER, Thrombospondin-2, MAGP-1/MF AP2, Thrombospondin-3, MAGP- 2/MF AP5, Thrombospondin-4, and Netrin-1. In some embodiment, the ligand may be JAG1; JAG2 and/or Delta-like-1. In some aspects, the ligand may be VCAM1. Shukla S et al. and Michaels et al. have shown that both DLL4 and vascular cell adhesion molecule (VCAM1) play an important role in the differentiation of effector cells (e.g., T cells) (see Shukla et al. Nature Methods. 2017; 14(5):531-8 and Michaels Y S, et al. bioRxiv. 2021.; the contents of each of which are herein incorporated by reference in their entirety). In some embodiment, the ligand may be modulator of apoptosis, such as Fas Ligand (FasL) The Fas-receptor/Fas ligand pathway has been shown to play crucial role in tolerance to self-antigen, and expression of Fas-L at transplant site induced tolerance of allogenic tissues (see Ji Lei et al. Sci Adv, 8, 2022; Lau et al Science 1996 273:109). In some embodiments, the agent may be a growth factor. Exemplary growth factors include without limitation vascular endothelial growth factor (VEGF), bone morphogenetic protein(s) (BMP), a transforming growth factor (TGF) such as transforming growth factor beta, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF), GCSF, Erythropoietin, TPO, GDF, neurotrophins, MSF, SGF, GDF, activin, CTGF, Epigen, Galectin, KGF, leptin, MMIF, MIA (melanoma inhibitory activity), myostatin, noggin, NOV, omentin, Oncostatin-M, Osteopontin, OPG, periostin, placental growth factor, placental lactogen, prolactin, RANK ligand, retinol binding protein (RBP), stem cell factor, amphiregulin, lymphocyte function associated Antigen-3, myeloid derived growth factor, osteoclast stimulating factor, progranulin, colony stimulating factor and combinations thereof. Growth factors may be one or more of stem cell factor (SCF), granulocyte-colony stimulating factor (G-CSF), granulocyte-macrophage stimulating factor (GM-CSF), stromal cell-derived factor-1, steel factor, VEGF, TGFb, platelet derived growth factor (PDGF), angiopoetins (Ang), epidermal growth factor (EGF), bFGF, HNF, NGF, fibroblast growth factor (FGF), hepatocye growth factor, liver growth factor (LGF) insulin-like growth factor (IGF-1), colony-stimulating factors, thrombopoietin, erythropoietin, fit3-ligand, tumor necrosis factor a (TNFa), a growth factor of the bone morphogenetic protein (BMP) family (e.g. BMP1, BMP2, BMP3, BMP4, BMP5, BMP6, BMP7, BMP8a, BMP8b, BMP10, BMP11, BMP15), a growth factor that functions in the Wnt signaling pathway (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT 16), and other growth factors.

In some embodiments, the ligand may promote long term survival of the thymic cells of the disclosure. For example, the ligand that promotes the long term survival of thymic cells may be FGF2, FGF7, RANKL (see Lee H-W et al. Expt & Mol. Med. 2008; 40(1):59-70; the contents of which are herein incorporated by reference in its entirety). In some embodiments, the ligands may promote vascularization e.g., VEGF (see de Barros S C. The Journal of Immunology. 2020; 205(9):2423-36; and Chung B, et al. Stem Cells, 2014; 32(9):2386-96; the contents of each of which are herein incorporated by reference in their entirety).

In some embodiments, an agent may be an immune modulator. Suitable immuno modulators include, but are not limited to, prednisone, azathioprine, 6-MP, cyclosporine, tacrolimus, methotrexate, interleukins (e.g., IL-2, IL-7, IL-6, IL-3, IL-la, IL-Ib, IL-6, IL-7, IL-8, IL-11, IL-13, IL-12), cytokines, chemokines, cytosine phosphateguanosine, oligodeoxynucleotides, glucans, antibodies, and aptamers. In some embodiments, one or more of the one or more agents is a chemokine. Exemplary chemokines include, without limitation, e.g., CCL3, CCL26, CXCL13, CXCL14, CCL6, CCL27, CXCL16, CXCL17, CXCL6, CXCL5, eotaxin, CCL2, CX3CL1, CXCL1, 2, 3, CCL14, CCL1, CXCL8, CXCL11, CC3L1, XCL1, CCL2, 7, 8, 12, 13, CCL22, CCL28, CXCL9, CCL3, 4, 9, 15, CXCL7, CCL4, CXCL4, CXCL12, CCL17, CCL21, CCL25, CCL16, FAM19A5, CXCL15, and any combination thereof. In some embodiments, one or more of the one or more agents is a cytokine. Exemplary cytokines include, without limitation, interferons (e.g. IFN-a, IFN-J3, IFN-s, IFN-K, IFN-CO, and IFN-y), granulocyte colony-stimulating factor, imiquimod, 4-1BB, adiponectin, AITR, AIFl, B-cell activating factor, beta defensin, betacellulin, BMP, BST1, B type Natriuretic peptide, cardiotrophin, CTLA4, EBB, Endoglin, epiregulin, FAS, Flt3 ligand, follistatin, hedgehog protein, interferons (e.g. interferon alpha, interferon gamma, interferon tau, interferon beta, interferon regulatory factor), interleukins (e.g., IL-1, IL-2, IL-3, IL-4, IL-S, IL-6, IL-7, IL-8, IL-9, IL-10, IL-11, IL-12, IL-13, IL-14, IL-15, IL-16, IL-17, IL-8, IL-19, IL-20, IL-21, IL-22, IL-23, IL-24, IL-27, IL-28A, IL-29, IL-31, IL-32, IL-33, IL-34, IL-35, IL-36, IL-37), otoraplin, resistin, leukemia inhibitory factor, serum amyloid A, TPO, trefoil factor, thymic stromal lymphopoietin, tumor necrosis factor, uteroglobin, visfatin, wingless-type MMTV nitration site family, AIMP1, CLCF1, CYTL1, EMAP II, TAFA2, Vaspin, and any combination thereof. In some embodiments, the immune modulator may be an agent such as CXCL12 that promotes migration of dendritic cells towards or away from the cells and/or TSSs of the disclosure (see Ramos S A, et al. Journal of Allergy and Clin. Imm. 2021; the contents of which are herein incorporated by reference in its entirety). In some embodiments, the immuno modulator may be a ligand for CCR7 and/or CCR9 that may promote the recruitment of effector cells, such as T cells to the thymic cells or the TSSs of the disclosure (Gameiro J et al. Cell adhesion & migration. 2010; 4(3):382-90; the contents of which are herein incorporated by reference in its entirety). In some embodiments, the immuno modulator may be thymic stromal lymphopoietin (TSLP) and/or IL10 that may promote the development of effector T cells e.g., regulatory T cells (see Alawam A S, et al. Frontiers in immunology. 2020; 11:858; the contents of which are herein incorporated by reference in its entirety).

In some embodiments, the agent may be a hormone. Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol). Exemplary hormones include, without limitation, endothelin, exendin, follicle stimulating hormone, growth hormone releasing hormone, growth hormone releasing peptides, ipamorelin, glucagon, glucagon-like peptides, insulin, chorionic gonadotropin, inhibin-Beta C Chain, inhibin alpha, inhibin alpha chain, luteinizing hormone, luteinizing hormone releasing hormone, peptide hormones (e.g., adrenocorticotropic hormone, alarelin, antide, atosiban, buserelin, cetrorelix, desmopressin, deslorelin, elcatonin, ganirelx, ghrelin, goserelin, hexarelin, gistrelin, lanreotide, leuprolide, lypressin, melanotan-I and -II, nafarelin, octreotide, pramlintide, secretin, sincalide, somatostatin, terlipressin, thymopentin, triptorelin, vasopressin, neuropeptide Y, cholecystokinin), procalcitonin, prolactin, oxytocin, parathyroid hormone, estrogen, testosterone, stanniocalcin-1 and -2, thymosin, thyrostimulin, thyroid stimulating hormone, agouti-related protein, calcitonin, corticotrophin releasing hormone binding protein, prouroguanylin, oxyntomodulin, thyrotropin releasing hormone, and any combination thereof.

In some embodiments, the agents of the disclosure may promote angiogenesis.

An agent can be a gene modifying agent. Exemplary gene modifying agents include, but are not limited to, RNA guided or programmable nuclease systems such as a CRISPR-Cas system, Meganucleases, Zinc Finger Nucleases, and/or the like. Such systems are generally known in the art.

As used herein, “deoxyribonucleic acid (DNA)” and “ribonucleic acid (RNA)” generally refer to any polyribonucleotide or poly deoxyribonucleotide, which may be unmodified RNA or DNA or modified RNA or DNA. RNA can be in the form of non-coding RNA such as IRNA (transfer RNA), snRNA (small nuclear RNA), IRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

In some embodiments, an agent is a chemical agent. As used herein, “chemical agent” refers to a chemical substance, molecule, or composition. Exemplary chemical agents are those that are suitable for use as a pharmaceutical agent in an animal as well as those that are not. In some embodiments, the chemical agent is a hazardous chemical agent. In some embodiments, the chemical agent is not hazardous. In some embodiments, the chemical agent can be a carcinogen. In some embodiments, the chemical agent is biocompatible. The term “biocompatible,” as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

### Devices

Cells and/or TSSs of the disclosure may be incorporated into devices. Mechanical parameters, such as flow, shear stresses, pressure, and movements, can have an impact thymic cell culture, differentiation, and tissue homeostasis. For example, blood-flow-induced shear stresses on the cells can trigger terminal maturation. Similarly, diverse physical forces are involved in normal and pathologic function of cells, tissues, organs and/or organisms. Integration of the compositions of the disclosure within devices that allow the generation of forces and movements allowing the compositions to more closely mimic the thymic environment in vivo. Organ-on-a-chip technology has introduced many techniques for the generation of forces and their maintenance and may be useful in the present disclosure. Organ-on-a-chip-like devices also allow the growth of epithelia on 2D membranes with microfluidic channels on both apical and basal sides, providing bilateral accessibility and the ability to apply fluid flow. Such microfabrication-based devices provide the opportunity to combine biophysical and biochemical stimuli and, thus, increase the physiological relevance of in vitro models and the robustness of their generation protocols.

Microfluidic devices may be utilized to integrate access channels for waste removal and nutrient supply within populations of cells, and to enable independent control over experimental conditions.

Any of the microfluidic devices described in US Patent Publication US20210115369 and U.S. Pat. No. 10,829,727 may be useful in the present disclosure. The contents of each of these publications are hereby incorporated by reference in their entirety.

### Methods

The present disclosure provides methods of differentiating pluripotent stem cells into thymic cells. In some embodiments, the present disclosure provides method of differentiating induced pluripotent stem cells into thymic cells.

In some embodiments, the steps involved in the differentiation of iPSCs to thymic cells can include encapsulation of single iPSCs. In some other embodiments, the steps involved in the differentiation of iPSCs to thymic cells do not include encapsulation of single iPSCs.

In some embodiments, one or more of the steps involved in the differentiation of iPSCs to thymic cells can include the activation of WNT signaling. As a non-limiting example, the activator of WNT signaling can be CHIR99021.

In some embodiments, one or more of the steps involved in the differentiation of iPSCs to thymic cells can include the inhibition of WNT signaling. As a non-limiting example, the inhibitor of WNT signaling can be IWR1 (or IWR-1).

In some embodiments, one or more of the steps involved in the differentiation of iPSCs to thymic cells can include the inhibition of BMP signaling. In some embodiments, the inhibition of BMP signaling can be achieved using BMP pathway inhibitor, LDN193189.

In some embodiments, one or more of the steps involved in the differentiation of iPSCs to thymic cells can include the inhibition of SHH signaling. In some embodiments, the inhibition of SHH is achieved by using an SHH antagonist, SANT-1.

In some embodiments, one or more of the steps involved in the differentiation of iPSCs to thymic cells can include the inhibition TGFβ signaling. In some embodiments, the inhibition of TGFβ signaling is achieved by using TGFβ inhibitor, SB431542.

In some embodiments, one or more of the steps involved in the differentiation of iPSCs to thymic cells can include a cell culture medium containing Insulin Transferrin Selenium (ITS), knockout replacement serum (KSR), Penicillin Streptomycin (also referred to herein as “Pen Strep”) and non-essential amino acids (NEAA).

In one embodiment, cells of the disclosure are cultured in the presence of one or more polymers described herein. In one embodiment, the cells of the disclosure are encapsulated in the polymers of the disclosure. In one embodiment, the polymer is alginate.

In one embodiment, iPS cells are cultured in suspension and/or are encapsulated in alginate. In one embodiment, DE cells are cultured in suspension and/or are encapsulated in alginate. In one embodiment, AFE cells are cultured in suspension and/or are encapsulated in alginate. In one embodiment, VPE cells are cultured in suspension and/or are encapsulated in alginate. In one embodiment, TEP cells are cultured in suspension and/or are encapsulated in alginate. In one embodiment, TECs are cultured in suspension and/or are encapsulated in alginate.

In one embodiment, iPS cells are cultured in suspension and not encapsulated in alginate. In one embodiment, DE cells are cultured in suspension and not encapsulated in alginate. In one embodiment, AFE cells are cultured in suspension and not encapsulated in alginate. In one embodiment, VPE cells are cultured in suspension and are not encapsulated in alginate. In one embodiment, TEP cells are cultured in suspension and are not encapsulated in alginate. In one embodiment, TECs are cultured in suspension and are not encapsulated in alginate.

In one embodiment, iPS cells are cultured in suspension and not encapsulated. In one embodiment, DE cells are cultured in suspension and not encapsulated. In one embodiment, AFE cells are cultured in suspension and not encapsulated. In one embodiment, VPE cells are cultured in suspension and are not encapsulated. In one embodiment, TEP cells are cultured in suspension and are not encapsulated. In one embodiment, TECs are cultured in suspension and are not encapsulated.

In one embodiment, iPS cells are differentiated in suspension and/or are encapsulated in alginate. In one embodiment, DE cells are differentiated in suspension and/or are encapsulated in alginate. In one embodiment, AFE cells are differentiated in suspension and/or are encapsulated in alginate. In one embodiment, VPE cells are differentiated in suspension and/or are encapsulated in alginate. In one embodiment, TEP cells are differentiated in suspension and/or are encapsulated in alginate. In one embodiment, TECs are differentiated in suspension and/or are encapsulated in alginate.

In one embodiment, iPS cells are differentiated in suspension and not encapsulated in alginate. In one embodiment, DE cells are differentiated in suspension and not encapsulated in alginate. In one embodiment, AFE cells are differentiated in suspension and not encapsulated in alginate. In one embodiment, VPE cells are differentiated in suspension and are not encapsulated in alginate. In one embodiment, TEP cells are differentiated in suspension and are not encapsulated in alginate. In one embodiment, TECs are differentiated in suspension and are not encapsulated in alginate.

In one embodiment, iPS cells are differentiated in suspension and are not encapsulated. In one embodiment, DE cells are differentiated in suspension and are not encapsulated. In one embodiment, AFE cells are differentiated in suspension and are not encapsulated. In one embodiment, VPE cells are differentiated in suspension and are not encapsulated. In one embodiment, TEP cells are differentiated in suspension and are not encapsulated. In one embodiment, TECs are differentiated in suspension and are not encapsulated.

In one embodiment, alginate encapsulated iPS cells are differentiated in suspension to DE cells. In one embodiment, alginate encapsulated iPS cells are differentiated in suspension to DE and AFE cells. In one embodiment, alginate encapsulated iPS cells are differentiated in suspension to DE or AFE cells. In one embodiment, alginate encapsulated iPS cells are differentiated in suspension to DE, AFE, VPE, and thymic cells. In one embodiment, alginate encapsulated iPS cells are differentiated in suspension to DE, AFE, VPE, or thymic cells.

In one embodiment, alginate encapsulated DE and AFE cells are differentiated in suspension into VPE cells. In one embodiment, alginate encapsulated DE and AFE cells are differentiated in suspension into VPE and thymic cells. In one embodiment, alginate encapsulated DE or AFE cells are differentiated in suspension to VPE or thymic cells. In one embodiment, alginate encapsulated AFE and VPE cells are differentiated in suspension to thymic cells. In one embodiment, alginate encapsulated AFE or VPE cells are differentiated in suspension to thymic cells. In one embodiment, alginate encapsulated VPE cells are differentiated in suspension to thymic cells.

In one embodiment, non-encapsulated iPS cells are differentiated in suspension to DE cells. In one embodiment, non-encapsulated iPS cells are differentiated in suspension to DE and AFE cells. In one embodiment, non-encapsulated iPS cells are differentiated in suspension to DE or AFE cells. In one embodiment, non-encapsulated iPS cells are differentiated in suspension to DE, AFE, VPE, and thymic cells. In one embodiment, non-encapsulated iPS cells are differentiated in suspension to DE, AFE, VPE, or thymic cells.

In one embodiment, non-encapsulated DE and AFE cells are differentiated in suspension into VPE cells. In one embodiment, non-encapsulated DE and AFE cells are differentiated in suspension into VPE and thymic cells. In one embodiment, non-encapsulated DE or AFE cells are differentiated in suspension to VPE or thymic cells. In one embodiment, non-encapsulated AFE and VPE cells are differentiated in suspension to thymic cells. In one embodiment, non-encapsulated AFE or VPE cells are differentiated in suspension to thymic cells. In one embodiment, non-encapsulated VPE cells are differentiated in suspension to thymic cells.

### Methods of Alginate Encapsulation

In some embodiments, oil/water emulsion methods are used for alginate encapsulation. The emulsion encapsulation allows for even cell spatial distribution within the small microcapsule. Alginate solutions containing cells are stirred with nontoxic paraffin oil and then cross linked resulting in cell laden microcapsules. Using this technique, parameters such as stirring rate can allow for tuning of capsule size optimal for differentiation. In some embodiments, extrusion (drop) methods can be used for alginate encapsulation.

In one embodiment, the cells of the disclosure are encapsulated by extrusion. Autoclaved alginate solution is prepared with alginate, gelatin, HEPES, and/or NaCl. Cells can be resuspended in an alginate solution at varying concentrations e.g., 200,000 cells/mL. Solutions are loaded into a 1 ml syringe equipped with a 27-gauge needle tip. The syringe is then placed into a syringe pump and extruded into a CaCl₂ gelling bath at a predetermined rate. Alginate capsules are allowed to remain in the bath for an additional time. The capsules are then washed with PBS and suspended into Stem Scale media containing Y-27632. Cells are cultured for 1-5 days before differentiation.

In one embodiment, the cells of the disclosure are encapsulated by an emulsion method using autoclaved solutions of alginate, gelatin, HEPES, NaCl, and/or CaCO₃. Mineral oil can be added to a sterile containing a 35 mm stir bar magnetic. Cells are resuspended in an alginate solution at a varying concentration. Cell-alginate solution is added to the oil while stirring and is allowed to emulsify. After the emulsification, mineral oil containing acetic acid can be added and gelling is allowed to continue while stirring. After completion of gelling, culture media can be added to neutralize pH. Oil separation and capsule collection are achieved by centrifugation. Capsules were washed and resuspended in cell culture media for differentiation.

In some embodiments, cells of the disclosure are encapsulated in alginate. In one embodiment, single-cells are obtained by pretreating a confluent cell population with Y-27632 dihydrochloride (R&D Systems) prior to dissociating with Accutase (StemPro). In some embodiments, cells are suspended in 1.1 w/v % low viscosity alginate (Sigma) at a pre-determined density (e.g., 5×10⁵ per ml alginate). In some embodiments, the single-cell alginate mixture is polymerized dropwise with a stirred solution of CaCl₂) (for example, 100 mM CaCl₂)) with HEPES (e.g., 10 mM HEPES) to form capsules. In one embodiment, the capsules are spherical. In some embodiments, the capsules are washed three times with DMEM/F12 (Gibco) before culturing in mTeSRI (StemCell Technologies) supplemented with 10 μM Y-27632 dihydrochloride for 4-6 days before starting differentiation.

### Methods of Alginate Decapsulation

At any step of the differentiation, the encapsulated cells of the disclosure can be decapsulated, that is, the alginate encapsulation is removed. In one embodiment, the iPS cells are encapsulated in alginate and subsequently differentiated to thymic cells using methods described herein. Differentiated thymic cells are then decapsulated prior to implantation in vivo.

In one embodiment, cells of the disclosure are decapsulated in a solution containing sodium citrate, HEPES, and/or NaCl. Capsules are suspended in the de-encapsulation solution for while shaking to promote de-encapsulation. The aggregates are collected by centrifugation and then treated with accutase to dissociate into single cells. The single cells are collected by centrifugation and used for further testing.

In one embodiment, cells of the disclosure are decapsulated with EDTA. In one embodiment, the cells are placed in a Ficoll gradient to remove both capsular debris and dead cells. In some embodiments, the live cell layer is pipetted off the gradient and dissociated for analysis.

### Preparation and Maintenance of Thymic Cells

Provided herein are methods of differentiating pluripotent stem cells into thymic cells. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes DMEM-F12, Activin A, CHIR99021, insulin transferrin selenium (ITS), and knockout serum replacement (KSR). In some aspects, the second growth medium includes DMEM-F12, bFGF, Activin A, LDN193189, ITS and KSR. In some embodiments, the cells are cultured in the presence of PI-103. The concentration of PI-103 can be from about 1 nM to 1000 nM. In one embodiment, the concentration of PI-103 can be 50 nM.

The definitive endoderm cells can be further cultured and differentiated into anterior foregut cells by contacting or incubating the definitive endoderm cells with at least one of SB431542, LDN-193189 and KSR. In some embodiments, the anterior foregut cells can be cultured and differentiated into pharyngeal endoderm cells by contacting or incubating the anterior foregut cells with at least one of EGF, retinoic acid, FGF8B, and SHH. In some embodiments, the pharyngeal endoderm cells can be cultured and differentiated into, thymic epithelial cells by contacting or incubating the pharyngeal endoderm cells with at least one of BMP4, FGF8b, EGF, SANT, CHIR99021, Ascorbic Acid, or a combination thereof. In some embodiments, the differentiation is performed from about 14 days to 25 days. For example, the differentiation is performed for about 18 days, 19 days, 20 days, 21 days, 22 days, 23 days, 24 days, or 25 days.

In some embodiments, the present disclosure provides methods for the preparation of one or more cells or cell types described herein. In some embodiments, the cells can be thymic cells.

An accumulating body of data in public databases provide single cell transcriptomes of primary human and murine thymuses (See Bautista et al. 2021 Nat Commun 12, 1096; Kernfeld, et al. Immunity. 2018 Jun. 19; 48(6):1258-1270.e6; Zeng et al. Immunity. 2019 Nov. 19; 51(5):930-948.e6; the contents of each of which are herein incorporated by reference in its entirety). This provides a rich source of material for identification of factors that promote the differentiation and/or maturation of cells of the disclosure to thymic cells. By analysis of scRNA sequencing data, the present disclosure identifies potential factors and/or supporting cells that can promote and/or maintain thymic cell phenotype.

In some embodiments, cells of the present disclosure can be isolated from an organism. In some embodiments, the organism can be a mammal. Mammalian cells can be isolated from human, rodent, porcine and/or bovine sources. Human sources of cells of the disclosure can be autologous or allogeneic. In some embodiments, the tissues that contain the cells of the disclosure can be harvested and used as such for applications described herein. Cells of the disclosure can be obtained from embryonic, fetal, adult organism. In some aspects, the organism can be alive or can be a cadaver organism.

Cells described herein can be derived from other cell types. As a non-limiting example, the cells of the disclosure can be derived from pluripotent stem cells (PSCs). In some embodiments, the cells of the disclosure can be derived from progenitor cells. In some embodiments, cells of the disclosure can be derived by the differentiation of PSCs and/or progenitor cells.

In some embodiments, thymic cells can be prepared from PSCs. In this regard, the methods can comprise culturing the pluripotent stem cells for a time and under conditions sufficient to differentiate the pluripotent stem cells into thymic cells. For example, the method can comprise culturing the pluripotent stem cells in the presence of the factors and/or inhibitors that drive the differentiation of PSCs to thymic cells. Methods for differentiating PSCs into thymic cells are known in the art. The methods for differentiating PSCs into thymic cells can include the use of one or more parameters know in the art for differentiation or combinations thereof. The parameters include, but are not limited to, (i) factors promoting differentiation (ii) inhibitors promoting differentiation (iii) duration of time for promoting differentiation (iv) temperature (v) substrate and/or (vi) supporting cells that promote differentiation. Any of the methods or parameters for differentiating PSCs into thymic cells described in following references can be used herein and include Parent et al. Cell Stem Cell. 2013 Aug. 1; 13(2):219-29; Soh et al. Stem Cell Rep. 2014 Vol. 2 j 925-937; Sun et al. Cell Stem Cell. 2013 Aug. 1; 13(2):230-6; Okabe et al. Cell. Reprog. 2015 Vol 17, No. 5; Su et al. Sci. Rep. 2015 5, 9882; Otsuka et al. Sci Rep 2020 10:224; International Patent Publications, WO2019060336, WO2020205859, WO2020220040, WO2014134213, WO2010143529 WO2011139628; and Chinese Patent Publication CN201110121243; the contents of each of which are herein incorporated by reference in their entirety.

In one embodiment the iPS cells are encapsulated in polymers such described herein, such as alginate, prior to their differentiation into thymic cells.

### Preparation and Maintenance of Definitive Endoderm (DE) Cells

The present disclosure provides method for preparing definitive endoderm cells which can subsequently be differentiated into thymic cells. In some embodiments, the definitive cells can be prepared by culturing cells in two-dimensional culture or three-dimensional culture. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes Activin A, CHIR99021, insulin transferrin selenium (ITS), and/or knockout serum replacement (KSR). In one embodiment the iPS cells are encapsulated in polymers such described herein, such as alginate, prior to their differentiation. Encapsulated cells are grown in three dimensional cultures.

In some aspects, the second growth medium includes basic fibroblast growth factor (bFGF), Activin A, LDN193189, ITS and KSR. In some embodiments, the second growth medium includes CHIR99021.

In some embodiments, the concentration of CHIR99021 is from about 0.1 μM to 100 μM. In some embodiments, the concentration of CHIR99021 is about 2 μM-3 μM.

In some embodiments, the cells are cultured in the presence of PI-103. The concentration of PI-103 can be from about 1 nM to 1000 nM. In one embodiment, the concentration of PI-103 can be 50 nM. In some aspects, the concentration of PI-103 can be 25 nM. In some embodiments, the pluripotent stem cells can be cultured for about three to five days. The PI-103 can be added for 1-2 days. The pluripotent stem cells can be embryonic stem cells or induced pluripotent stem cells.

In some embodiments, the pluripotent stem cells can be cultured for about three to five days. Stem cells can be cultured in the first growth medium for about one to two days and in the second growth medium for about two to three days. The pluripotent stem cells can be cultured in the first growth medium for two days and in the second growth medium for three days. In some embodiments, the concentration of Activin A can be about 100 ng/ml. In some embodiments, the concentration of CHIR99021 can be 2 μM. In some embodiments, the concentration of bFGF can be 10 ng/ml. In some embodiments, the concentration of LDN193189 can be 200 nM. In some embodiments, CHIR99021 can be added to the second growth medium for about one day.

Provided herein are methods of differentiating pluripotent stem cells into thymic cells. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes DMEM-F12, Activin A, CHIR99021, insulin transferrin selenium (ITS), and knockout serum replacement (KSR). In some aspects, the second growth medium includes DMEM-F12, bFGF, Activin A, LDN193189, ITS and KSR. In some embodiments, the second growth medium includes CHIR99021. In some embodiments, the concentration of CHIR99021 is from about 0.1 μM to 100 μM. In some embodiments, the concentration of CHIR99021 is 2 μM. In some embodiments, the cells are cultured in the presence of PI-103. The concentration of PI-103 can be from about 1 nM to 1000 nM. In one embodiment, the concentration of PI-103 can be 50 nM.

### Preparation of Anterior Foregut Endodermal (AFE) Cells

The definitive endoderm cells can be further cultured and differentiated into anterior foregut cells. In some embodiments, the AFE cells can be prepared by culturing cells in two-dimensional culture or three-dimensional culture. Definitive endoderm cells can be differentiated into AFE cells by contacting DE cells with a BMP inhibitor, a TGFβ inhibitor, at least one FGF, and/or Ascorbic acid.

In one embodiment the cells are encapsulated in polymers such described herein, such as alginate, prior to their differentiation. Encapsulated cells are grown in three dimensional cultures.

In some embodiments the cell culture medium utilized for the differentiation of DE cells to AFE cells can include N2-supplement (GIBCO, Waltham, Massachusetts), Basal Medium Eagle (BME), GLUTAMAX (GIBCO, Waltham, Massachusetts), B27™ serum-free supplement, non-essential amino acids, KSR and/or ITS.

In some embodiments, the cell culture medium utilized for the differentiation of DE cells to AFE cells does not include B27™ serum-free supplement.

In some embodiments the BMP inhibitor can be LDN193189. In some embodiments, the concentration of LDN193189 is from about 0.1 nM to about 1000 nM. In some aspects, the concentration of LDN193189 is from about 100 to 200 nM.

In some embodiments, the TGFβ inhibitor can be SB431542. In some embodiments, the concentration of SB431542 is from about 1 μM to about 100 μM. As a non-limiting example, the concentration of SB431542 is 10 μM.

In some embodiments, the FGF can be FGF8. In some embodiments, the concentration of FGF8 is from about Ing/ml to about 100 ng/ml. As a non-limiting example, the concentration of FGF8b is about 25-50 ng/ml.

DE cells can be differentiated to AFE cells for about 1 day, 2 days, 3 days, 4 days, or 5 days.

### Preparation of Ventral Pharyngeal Endoderm (VPE) Cells

Differentiation of AFE to VPE cells is performed as a single step process or as a multistep process. The multi-step process can be a two-step process. In a first step, the AFEs are cultured in a VPE1 media and in the second step, the cells are cultured in a VPE2 media. In one embodiment the cells are encapsulated in polymers such described herein, such as alginate, prior to their differentiation. Encapsulated cells are grown in three dimensional cultures.

In some embodiments, the VPE cells can be prepared by culturing cells in two-dimensional culture or three-dimensional culture. The VPE1 step can include culturing cells for about 1 day, 2 days, 3 days, 4 days, or 5 days. The VPE2 step can include culturing cells for about 2 days, 3 days, 4 days, 5 days, or 6 days.

In some embodiments, the VPE1 media can include Retinoic Acid, at least one FGF, a WNT inhibitor, TGFβ inhibitor, and/or Ascorbic acid.

In some embodiments, the VPE2 media can include Noggin, BMP inhibitor, WNT activator (e.g., CHIR99021), at least one FGF, Retinoic Acid, an SHH antagonist, and/or Ascorbic acid.

In some embodiments, the FGF can be FGF8, FGF7, and/or FGF10. In some embodiments, the concentration of FGF8 is from about Ing/ml to about 100 ng/ml. As a non-limiting example, the concentration of FGF8b is about 25-50 ng/ml.

In some embodiments, the WNT inhibitor is IWR1. The concentration of IWR1 can be from about 0.01 to 10 μM. As a non-limiting example, the concentration of IWR1 is 2.5 μM.

In some embodiments, the TGFβ inhibitor can be SB431542. In some embodiments, the concentration of SB431542 is from about 1 μM to about 100 μM. As a non-limiting example, the concentration of SB431542 is 10 μM.

In some embodiments, the concentration of Ascorbic Acid is from about 0.1 to 30 μM. As a non-limiting example, the concentration of Ascorbic Acid can be 10 μM.

In some embodiments the BMP inhibitor can be LDN193189. In some embodiments, the concentration of LDN193189 is from about 0.1 nM to about 1000 nM. In some aspects, the concentration of LDN193189 is from about 100 to 200 nM.

In some embodiments, the SHH inhibitor can be SANT-1. In some embodiments, the concentration of SANT-1 is from about 0.01 μM to about 10 μM. As a non-limiting example, the concentration of SANT-1 is 0.25 μM.

In some embodiments, the anterior foregut cells can be cultured and differentiated into pharyngeal endoderm cells by contacting or incubating the anterior foregut cells with at least one of EGF, retinoic acid, FGF8B, and/or SHH.

In some embodiments the VPE1 and/or VPE2 media can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

### Preparation of Thymic Epithelial Progenitor (TEP) Cells

Differentiation of VPE cells to TEP cells can be performed by culturing the cells in a TEP media. In some embodiments, the TEP cells can be prepared by culturing cells in two-dimensional culture or three-dimensional culture. The TEP step can include culturing cells for about 1 day, 2 days, 3 days, 4 days, 5 days, or 6 days.

In one embodiment the cells are encapsulated in polymers such described herein, such as alginate, prior to their differentiation. Encapsulated cells are grown in three dimensional cultures.

In some embodiments, the VPE cells can be differentiated into TEP cells using BMP (e.g., BMP4, BMP2), a WNT activator e.g., CHIR99021, at least one FGF, and/or Ascorbic acid.

In some embodiments the TEP media can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

In some embodiments, the BMP can be BMP2 or BMP4. The concentration of BMP can be Ing/ml to about 100 ng/ml. In some aspects, the concentration of BMP can be 50 ng/ml.

In some embodiments, the FGF can be FGF8, FGF7, FGF1, and/or FGF10. In some embodiments, the concentration of FGF is from about 1 ng/ml to about 100 ng/ml. As a non-limiting example, the concentration of FGF is about 25-50 ng/ml.

In some embodiments, the pharyngeal endoderm cells can be cultured and differentiated into, thymic epithelial cells by contacting or incubating the pharyngeal endoderm cells with at least one of BMP4, FGF8b, EGF, SANT-1 (SHH antagonist), CHIR99021, Ascorbic Acid, or a combination thereof.

### Preparation of Thymic Epithelial Cells (TEC)

TEP cells can be further differentiated in vitro into TECs. Differentiation to TECs can be performed in 2D or 3D culture. In some embodiments, the differentiation of TEPs can be performed for about 2 days, 3 days, 4 days, 5 days, 6 days, or 7 days.

In one embodiment the cells are encapsulated in polymers such described herein, such as alginate, prior to their differentiation. Encapsulated cells are grown in three dimensional cultures.

In some embodiments, the differentiation of TEPs to TECs is carried out in a TEC medium.

TEC medium can include RANKL, Interleukin e.g. (IL22), at least one FGF, at least one BMP (e.g., BMP4), a WNT activator, and/or ascorbic acid.

In some embodiments, the concentration of RANKL can be from about Ing/ml to about 100 ng/ml. In some aspects, the concentration of RANKL can be from about 20 ng/ml to about 50 ng/ml.

In some embodiments, the FGF can be FGF8, FGF7, FGF1, and/or FGF10. In some embodiments, the concentration of FGF is from about Ing/ml to about 100 ng/ml. As a non-limiting example, the concentration of FGF is about 25-50 ng/ml.

In some embodiments, the concentration of interleukins is from about Ing/ml to about 100 ng/ml. As a non-limiting example, the concentration of IL22 is about 20 ng/ml.

The TEC medium can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

In some embodiments, the differentiation is performed from about 14 days to seventeen days. In some embodiments, the cells of the disclosure can be cultured as aggregates. In some aspects, the cells disclosure can be cultured in an extracellular matrix-based medium e.g., Geltrex.

### Preparation of Effector Cells

Also provided herein are methods of preparing effector cells. In some embodiments, the effector cells can be lymphocytes. Effector cells can be obtained from primary cells from a mammal or from an established cell line. If obtained from a mammal, the effector cells can be obtained from numerous sources, including but not limited to blood, bone marrow, lymph node, thymus, spleen, or other tissues or fluids. Effector cells can also be enriched for or purified. In some embodiments, the effector cells can be T cells. The T cells can be any type of T cells and can be of any developmental stage, including but not limited to, CD4+/CD8+ double positive T cells, CD4+ helper T cells, e.g., Th1 and Th2 cells, CD4+ T cells, CD8+ T cells (e.g., cytotoxic T cells), peripheral blood mononuclear cells (PBMCs), peripheral blood leukocytes (PBLs), tumor infiltrating cells (TILs), memory T cells, naïve T cells. Methods of isolating and/or enriching lymphocytes are known in the art. Methods of enriching a population of lymphocytes obtained from a mammal or a donor can be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), and/or bead-based column separation.

In some embodiments, effector cells described herein can be derived from pluripotent stem cells. In some embodiments, effector cells can be derived from embryonic stem cells, hematopoietic stem or progenitor cells, cells isolated from bone marrow, cord blood, peripheral blood, thymus, or the stem or progenitor cells can have been differentiated from embryonic stem cells (ESC) or induced pluripotent stem cells (iPSC) in vitro. Stem or progenitor cells from primary tissue or ESC or iPSC can be from human or non-human animals (e.g., mouse) in origin.

In some embodiments, effector cells can be prepared differentiating the pluripotent stem cells or progenitor cells into lymphocytes by culturing PSC or progenitor cells with supporting cells that ectopically express a Notch ligand. In some embodiments, the supporting cells can be OP9 cells. In some embodiments, the Notch ligand is Delta-like 1 (DLL1). In some embodiments, the Notch ligand is Delta-like 4 (DLL4). In some embodiments, the Notch ligand is one described herein or in the art, such as in U.S. Pat. No. 7,795,404, which is herein incorporated by reference in its entirety. Effector cells of the present disclosure can be prepared using the Artificial Thymic Organoid (ATO) cell culture system which utilizes supporting cells that ectopically express OP9-DLL1. In some embodiments, the method further comprises contacting the co-cultured stem or progenitor cells and stromal cells with Flt-3 ligand and/or IL-7 and/or Stem Cell Factor/Kit ligand and/or thrombopoietin. In some embodiments, differentiating the stem or progenitor cell into a T cell comprises: culturing a three dimensional (3D) cell aggregate, comprising: a) a selected population of supporting cells that express an exogenous Notch ligand; b) a selected population of stem or progenitor cells; with a serum-free medium comprising B-27® supplement, xeno-free B-27® supplement, GS2 l ™ supplement, ascorbic acid, Flt-3 ligand, IL-7, or a combination thereof. Any of the methods for generating lymphocytes from stem cells or progenitor cells described in International Patent Publication WO2017075389 can be useful in the present disclosure (the contents of which are herein incorporated by reference in their entirety).

In some embodiments, the effector cell can be or can be derived from a hematopoietic cell. Methods of preparing hematopoietic cells from pluripotent stem cell are known in the art, for example as described in U.S. Pat. No. 9,834,754 and can include one or more of the following steps such as (i) inducing hematopoietic differentiation in a population of human pluripotent stem cells, wherein activin/nodal signaling is inhibited between day 1 and day 4 of differentiation; (ii) sorting the induced population based on expression of CD34 and CD43; and/or (iii), selecting a fraction of the cell population that is CD34 positive and/or CD43 negative and wherein the sorting and cell fraction selection is performed on a day selected from about day 6 to day 11 of differentiation (the contents of U.S. Pat. No. 9,834,754 are herein incorporated by reference in its entirety).

In some embodiments, thymic cells and/or effector cells can be cultured in the presence of extracellular vesicles (e.g., exosomes) derived from thymic cells and/or effector cells. Methods of preparing exosomes of thymic cells are described in US Patent Publication US2020299641 and (the contents of which are herein incorporated by reference in its entirety). In some embodiments, the exosomes can be derived from thymic cells engineered to ectopically express DLL1.

In some embodiments, effector cells such as T cells, can be derived by differentiation of other cell types. T cell differentiation can include four stages: 1) Mesoderm induction (at about days 1-4), 2) hematopoietic specification (at about days 4-8) 3) hematopoietic commitment and expansion (at about days 8-10), and/or 4) T-lymphoid differentiation. PSCs (iPSCs or ESCs) can be utilized as the starting cell population for mesoderm differentiation. These cells can be differentiated into mesoderm cells. The mesoderm cells can further be differentiated into hematopoietic cells which can be expanded in cell numbers. The cell culture systems for use in the present disclosure include, but are not limited to, a first cell culture media for mesoderm induction, a second cell culture media for hematopoietic specification and expansion, and a third cell culture media for T-lymphoid differentiation. The first cell culture media can include BMP4 (e.g., human BMP4) and bFGF (e.g., human bFGF). PSCs or ESCs can be used as the starting cell population. Undifferentiated PSCs or ESCs can be transferred to low-attachment plates to allow for the formation of embryoid bodies (EBs). The formation of EBs during the first stage can be facilitated by an overnight incubation in the presence of hBMP4. EBs can then be cultured with BMP4 and bFGF until day 4 to allow for mesoderm induction. The successful induction of mesoderm can be tested by, e.g., by measuring the percentage of KDR+PDGFR− cells. The second cell culture media can include VEGF (e.g., hVEGF), and a cocktail of hematopoietic cytokines. The cocktail of hematopoietic cytokines can include SCF (e.g., hSCF), Flt3L (e.g., hFlt3L), at least one cytokine, and bFGF for hematopoietic specification. The cytokine can be a Th1 cytokine, which includes, but is not limited to IL3, IL15, IL7, IL12 and IL21. EBs can be cultured in the second cell culture media for hematopoietic specification until about day 10. The EBs can be immunophenotypically analyzed by FACS for expression of CD34, CD31, CD43, CD45, CD41a, c-kit, Notch1, IL7Ra. In some embodiments, CD34+ cells from about day EBs express the highest levels of key transcription factors for lymphoid differentiation, e.g., CD127 (IL7Ra) and Notch1. The third cell culture media can include a feeder cell and SCF, Flt3L and at least one cytokine. The cytokine can be a Th1 cytokine, which includes, but is not limited to, IL3, IL15, IL7, IL12 and IL21. In some embodiments, at about day 10, the EBs can be dissociated, and the hematopoietic precursors can be transferred onto a feeder cell to induce T-lymphoid differentiation in an established co-culture system in the presence of the SCF, Flt3L and Th1 cytokine(s) (e.g., IL-7). In some embodiments, co-culture system can include thymic cells and/or feeder cultures, e.g., OP9-DLII feeder cells.

In some embodiments, co-culture can be performed using a co-culture medium. In some embodiments, the co-culture medium can include StemSpan SFEM II and StemSpan™ T Cell Progenitor Maturation Supplement. In some embodiments, the co-culture medium can include αMEM, 4% B27 supplement, 30 μM Ascorbic acid, 50 ng/ml IL7, 50 ng/ml FLT3L, 50 ng/ml TPO, 50 ng/ml SCF, and/or 1× Pen Strep. In some embodiments, the co-culture medium can include DMEM/F12, 1% B27 supplement without vitamin A, 50 μM Ascorbic acid, 50 ng/ml FGF8b, 50 ng/ml BMP, 50 ng/ml FGF10, 2 uM CHIR99021, 0.1% ITS, 0.0025% KSR, 0.5× Pen Strep, 1×NEAA, 1% N2, 1% Glutamax, 1% β-ME, 50 ng/ml IL7, 50 ng/ml FLT3L, 50 ng/ml TPO, and/or 50 ng/ml SCF.

In some embodiments, progenitors of effector cells are co-cultured with thymic cells of the disclosure to promote their differentiation. The co-culture can further include TSS and one or more supporting cells.

### Aggregate Size

In some embodiments, cells of the disclosure can be cultured in 3-dimensional culture. In some embodiment, cells of the disclosure can be in the form of aggregates or spheroids. The term “spheroid” refers to clusters of cells and/or cell colonies. Spheroids can be formed from various cell types, for example, thymic cells, pluripotent cells, effector cells, stem cells, and/or supporting cells. Spheroids can have sphere-like or irregular shapes. Spheroids can contain heterogeneous populations of cells, cell types, cells of different states, such as proliferating cells, quiescent cells, and necrotic cells.

In some embodiments, spheroid/aggregate size can be tuned. For example, aggregate size in pluripotent stem cells can be critical during expansion period since the size of the aggregate can determine oxygen distribution within the cell spheroid resulting in discrete zones composed of outside, middle, and inside spheroid regions along the oxygen supply from high to low, exhibiting proliferating, quiescent viable and apoptotic core property, respectively (Langan et al. Plos One. 2016; 11(2); the contents of which are herein incorporated by reference in its entirety). In some embodiments, the aggregates can be from about 50 μm to 500 μm, from about 100 μm to 1000 μm, from about 200 μm to 2000 μm, from about 250 μm to 2500 μm, from about 300 μm to 3000 μm, from about 400 μm to 4000 μm. In embodiment, the spheroid/aggregate size can be 250 μm.

The present disclosure provides methods of treating or preventing a condition in a subject. The methods may include administering to the mammal any of the populations of cells described herein, or a pharmaceutical composition comprising any of the populations of cells described herein, in an amount effective to treat or prevent the condition in the subject. The condition may be a cancer, an immunodeficiency, an autoimmune condition, an infection, or a blood condition.

### Thymic Indications

Thymic cells, effector cells and/or the compositions described herein may be used in the treatment or prevention of one or more diseases or indications associated with the absence, decline or aberrant functioning of the thymus of the subject. In some embodiments, thymic cells, effector cells and/or compositions described herein may be used in the treatment of athymia, a condition in which a subject may be born with a severely deficient thymus or wherein the thymus is completely absent in the subject. Conditions associated with athymia include, but are not limited to complete or partial Di George syndrome, complete or partial CHARGE syndrome, thymomas, thymus cancer, type A thymoma, type B thymoma, thymic atrophy, age-related thymic atrophy, thymic cyst, thymic hyperplasia, thymic hypoplasia, thymic aplasia, thymic dysplasia, thymic irradiation, myasthenia gravis, thymic carcinoma, thymic hyperplasia, thymic irradiation, age or infection associated decline in thymic function, thymic cells, effector cells and/or compositions may be used for the treatment of athymia associated with mutations, defects or deletions in genes involved in the development of thymic tissue, thymic cells, effector cells and/or compositions may be used for the treatment of athymia associated with mutations, defects, or deletions in PAX1 gene (herein referred to as PAX1 deficiency), thymic cells, effector cells and/or compositions may be used for the treatment of athymia associated with mutations, defects, or deletions in TBX1 gene (herein referred to as TBX1 deficiency), thymic cells, effector cells and/or compositions may be used for the treatment of athymia associated with mutations, defects, or deletions in FOXN1 gene (herein referred to as FOXN1 deficiency).

In some embodiments, thymic cells, effector cells and/or compositions described herein may be used in the treatment of thymic insufficiency related to advanced age, chemotherapy, radiotherapy, immunosuppressive drug treatment, graft-vs-host disease, T cell-depleted hematopoietic stem cell transplant and/or HIV infection.

### Surgery

In certain embodiments, the thymic cells, effector cells and/or compositions described herein may be used subject to treat a subject who may undergo a thymectomy surgery. In certain embodiments, the subject may have a congenital heart defect and may have received or is receiving an open heart surgery. A subject may undergo thymectomy for the treatment of one or more indications associated with the thymus e.g., myasthenia gravis or thymoma.

### Immunodeficiency

In some embodiments, compositions of the present disclosure may be used to treat immunodeficiency. The term immunodeficiency may refer to any condition in which a subject's immune system is compromised and/or in need of reconstitution, e.g., after irradiation or chemotherapy. Immunodeficiency may be a primary immunodeficiency, caused by an inherited or a genetic factor or a secondary immunodeficiency, caused by an environmental factor. In some embodiments, compositions of the present disclosure may be used to treat primary immunodeficiencies such as, but not limited to, Wiscott-Aldrich syndrome, severe combined immunodeficiency disease (SCID), DiGeorge syndrome, ataxia-telangiectasia, chronic granulomatous disease, transient hypogammaglobulinemia of infancy, agammaglobulinemia, complement deficiencies, T cell lymphopenia, and/or selective IgA deficiency. In some embodiments, compositions of the present disclosure may be used to treat secondary immunodeficiencies caused by diseases such as AIDS and/or hepatitis.

Lymphopenia as used herein, refers to a condition in which there is a lower-than-normal number of lymphocytes (a type of white blood cell) in the blood. When the lymphopenia is associated with a reduction in the number of T cells, it may be referred to as T cell lymphopenia. Compositions of the disclosure may be used to treat inherent or acquired lymphopenia which may be caused by hematopoietic stem cell therapy, bone marrow transplantation therapy, radiation, chemotherapy, surgery, immunosenescence and/or aging.

### Cancer

Various cancers may be treated with pharmaceutical compositions of the present disclosure. As used herein, the term “cancer” refers to any of various malignant neoplasms characterized by the proliferation of anaplastic cells that tend to invade surrounding tissue, metastasize to new body sites, and refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of carcinomas which may be treated with the compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma, Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

### Autoimmune Diseases

In some embodiments, thymic cells, effector cells, or any of the compositions described herein may be useful in the treatment of autoimmune diseases. Autoimmune diseases may arise in a subject when self-antigen(s) is/are recognized by the effector cells in extra-thymic tissue and/or when such recognition triggers an activated immune response in a subject. The present disclosure provides methods of preparing an effector cell capable of effecting an immune tolerance response in a subject. Effector cells and pharmaceutical compositions including the same may be useful in training the immune system of the subject. In some embodiments, engineered thymic cells of the present disclosure may also be administered to a subject for the treatment of autoimmune diseases.

In some embodiments, the compositions of the present disclosure may be used to treat type 1 autoimmune polyglandular syndrome (APS-1) or autoimmune polyendocrinopathy-candidiasis-ectodermal dystrophy (APECED) syndrome. More than 60 mutations of the autoimmune regulator (AIRE) gene are associated with the development of type 1 autoimmune polyglandular syndrome (APS-1). AIRE plays an important part in shaping the T-cell repertoire through its role in the elimination of T cells that are reactive to self-antigens in the thymus. The clinical manifestations associated with APS-1 classically involve mucocutaneous candidiasis, hypoparathyroidism, and adrenal insufficiency, chronic mucocutaneous candidiasis, hypoparathyroidism, hypergonadotropic hypogonadism, ovarian failure, and/or autoimmune hepatitis. In some embodiments compositions of the present disclosure may be administered with the current standard of care therapies of APS-1, such as, but not limited to, lifelong anti-fungals, calcium modulators, endocrine hormone replacement, corticosteroids with or without 5 azacytidine, 5 azacytidine with mycophenolate (autoimmune hepatitis), and/or 5 azacytidine with or without rituximab (autoimmune pneumonitis).

Autoimmune diseases may be rheumatoid arthritis multiple sclerosis, inflammatory bowel disease and allergic encephalomyelitis (EAE), systemic lupus erythematosus, rheumatoid arthritis, graft versus host disease, autoimmune pulmonary inflammation, autoimmune encephalomyelitis, Guillain-Barre syndrome, autoimmune thyroiditis, insulin dependent diabetes mellitus, Crohn's disease, scleroderma, psoriasis, Sjögren's syndrome, autoimmune inflammatory eye disease, primary biliary cirrhosis, Sjögren's syndrome, Temporal arteritis, Ulcerative Colitis, Vasculitis, Wegener's granulomatosis, Mixed Connective Tissue Disease, myasthenia gravis, narcolepsy, Guillain-Barré syndrome, Celiac disease, alopecia areata, polymyalgia, asthma, and Hashimoto's disease.

In some embodiments, the autoimmune disease may be immuno dysregulation polyendocrinopathy enteropathy X-linked syndrome (IPEX).

In some embodiments, the compositions of the present disclosure may be used in combination with a second therapeutic agent for the treatment of autoimmune diseases. The second therapeutic agents may be immunosuppressants or anti-inflammatory agents, which may include, but are not limited to, alkylating agent, an antimetabolite, a cytotoxic antibiotic, a folic acid analog, a purine analog, an antibody, a TNF binding protein, an interferon, an opioid, a mycophenolate, and a calcineurin inhibitor. Compositions of the present disclosure may also be used in combination with peripheral lymphodepletion.

In some aspects, thymic cells may be engineered to express MHC molecules, whose expression may be associated with a disease or a disorder. In some embodiments, the expression of the MHC molecules may be protective against a particular disease or disorder in a subject. In some aspects, the disease or disorder may be an autoimmune disease.

In some aspects, the MHC haplotypes expressed by thymic cells of the disclosure may be associated with a disease or a disorder in a subject. In some embodiments, the expression of the MHC haplotype may be protective against a particular disease or disorder. As a non-limiting example, thymic cells may be engineered to express DR1501-DQ6 haplotype as therapeutic strategy for the treatment and/or prevention of type I diabetes in a subject (see Wen et al., Science Immunology 5.44 (2020); the contents of which are herein incorporated by reference in their entirety).

### Cell, Tissue, and Organ Transplantation

In some embodiments, cells, compositions, and pharmaceutical compositions of the present disclosure may be used to improve the acceptance of cell, tissue, or organ transplantation and/or prevent the rejection of a cell, tissue, or organ transplantation. In some embodiments, the cells, compositions and/or pharmaceutical compositions of the present disclosure may be used to treat or prevent graft versus host disease (GvHD). In some embodiments thymic cells of the present disclosure may be administered to the subject prior to cell, tissue, or organ transplantation. Pre-grafting of thymic cells may result in an immune tolerance response in the subject. In some aspects, the compositions may be administered in combination with therapeutic agents known to suppress the immune system. Non limiting examples of immune suppressive therapeutic agents include calcineurin inhibitors, e.g., tacrolimus, cyclosporine; antiproliferative agents, e.g., mycophenolate mofetil, mycophenolate sodium and azathioprine; mTOR inhibitors, e.g., sirolimus and/or steroids, e.g., prednisone.

In some embodiments, the cells of the disclosure, e.g., thymic cells may be administered concurrently with transplantation of the cell, tissue, or organ, or after the cell, tissue, or organ transplantation has occurred.

In some embodiments, the organ transplant may be a solid organ transplantation (SOT) which represents a treatment modality for end-stage organ failure of the kidney, liver, pancreas, heart, and lung. SOT may also include small intestine and vascularized composite allografts.

In some embodiments, the organ transplant may be a hematopoietic stem cell transplant (HSCT). Non-limiting examples of indications requiring HSCT include, non-malignant indications such as, aplastic anemia, Fanconi anemia, Diamond-Blackfan syndrome, sickle cell disease, Thalassemia, Paroxysmal nocturnal hemoglobinuria, Chediak-Higashi syndrome, Chronic granulomatous disease, Glanzmann thrombasthenia, Osteopetrosis, Lysosomal storage disorders, Gaucher disease, Niemann-Pick, Mucopolysaccharidosis, Glycoproteinoses, Immune deficiencies, Ataxia telangiectasia, DiGeorge syndrome, Severe combined immunodeficiency (SCID), Wiscott-Aldrich, Kostmann syndrome, Shwachman-Diamond syndrome. Non-limiting examples of indications requiring HSCT include, malignant indications such as, but not limited to, leukemias such as acute myelogenous leukemia, acute lymphoblastic leukemia, hairy cell leukemia, chronic lymphocytic leukemia, myelodysplasia; lymphomas such as Hodgkin disease, Non-Hodgkin lymphoma, multiple myeloma, myeloproliferative neoplasms, myelofibrosis, myelofibrosis, chronic myelogenous leukemia; solid tumors such as neuroblastoma, desmoplastic small round cell tumor, Ewing sarcoma, and/or choriocarcinoma.

In some embodiments, the tissue transplantation may be a bone marrow transplantation (BMT).

Compositions, and cells e.g., thymic cells of the disclosure may be used to treat T cell lymphopenia observed following hematopoietic stem cell transplantation or bone marrow transplantation.

### Infectious Diseases

Cells, compositions, and pharmaceutical compositions of the disclosure may be used to treat infectious diseases. Infectious disease causing organisms include, but are not limited to, any one or more bacterial species (spp.) including, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, Enterococcus faecum), Escherichia spp. (e.g., Escherichia coli). Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Porphyromonas spp. (e.g., P. Gingavalis), Pseudomonas spp. (e.g., Pseudomonas aeruginosa), Rickettsia spp. (e.g., Rickettsia rickettsii), Salmonella spp. (e.g., Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (Yersinia pestis). Additional microorganisms causing infectious diseases may include, for example, one or more parasitic organisms (spp.) (e.g., parasite target(s)) including, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp., Hymenolepis spp. (e.g., H. nana, H. diminuta), Leishmania spp., Loa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, and/or Wuchereria bancrofti.

### Pharmaceutical Compositions

Pharmaceutical compositions of the present disclosure may include compositions with one or more cells described herein, and one or more pharmaceutically or physiologically acceptable carriers, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect prepared for intravenous administration.

In some embodiments, pharmaceutical formulations may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier may be supplemented with human serum albumin.

In some embodiment, the pharmaceutical composition may be substantially free of, e.g., there are no detectable levels of a contaminant, e.g., selected from endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiment, the bacterium is at least one selected from of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

### Buffers

In some embodiments, pharmaceutical compositions of the present disclosure are prepared with one or more buffering agents.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or combinations thereof.

Non-limiting examples include aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al., the contents of which are incorporated herein by reference in their entirety.

In some embodiments, pharmaceutical compositions of the present disclosure are formulated in parenteral dosage forms. The parenteral formulations may be aqueous solutions containing carriers or excipients such as salts, carbohydrates, and buffering agents (e.g., at a pH of from 3 to 9), or sterile non-aqueous solutions, or dried forms which may be used in conjunction with a suitable vehicle such as sterile, pyrogen-free water. For example, an aqueous solution of the therapeutic agents of the present disclosure comprises an isotonic saline, 5% glucose or other pharmaceutically acceptable liquid carriers such as liquid alcohols, glycols, esters, and amides, for example, as disclosed in U.S. Pat. No. 7,910,594 to Vlahov et al. (Endocyte), the contents of which are incorporated herein by reference in their entirety. In another example, an aqueous solution of the therapeutic agents of the present disclosure comprises a phosphate buffered formulation (pH 7.4) for intravenous administration as disclosed in Example 23 of WO2011014821 to Leamon et al., the contents of which are incorporated herein by reference in their entirety. The parenteral dosage form may be in the form of a reconstitutable lyophilizate comprising the dose of the therapeutic agents of the present disclosure. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

### Nutrients

In some embodiments, the pharmaceutical compositions of the present disclosure include one or more nutrients that promote the health, survival, and/or proliferation of the cells described herein.

In some embodiments, the pharmaceutical formulations include vitamins. In some embodiments, the pharmaceutical compositions include 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B 12, or the pharmaceutical compositions includes combinations thereof or salts thereof. In some embodiments, the pharmaceutical compositions include or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B 12. In some embodiments, the vitamins include or consist essentially of biotin, DL alpha tocopherol acetate, DL alpha-tocopherol, vitamin A, or combinations or salts thereof.

In some embodiments, the pharmaceutical compositions further include proteins. In some embodiments, the proteins include albumin or bovine serum albumin, a fraction of BSA, catalase, insulin, transferrin, superoxide dismutase, or combinations thereof. In some embodiments, the pharmaceutical compositions include one or more of the following: corticosterone, D Galactose, ethanolamine, glutathione, L-carnitine, linoleic acid, linolenic acid, progesterone, putrescine, sodium selenite, or triodo-I-thyronine, or combinations thereof.

In some embodiments, the pharmaceutical compositions include amino acids, inorganic ions, and/or monosaccharides. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions include sodium, potassium, calcium, magnesium, nitrogen, or phosphorus, or combinations or salts thereof. In some embodiments, the pharmaceutical compositions further include one or more of the following: molybdenum, vanadium, iron, zinc, selenium, copper, or manganese, or combinations. In some embodiments, the pharmaceutical compositions further include one or more of the following: corticosterone, D-Galactose, ethanolamine, glutathione, L-carnitine, linoleic acid, linolenic acid, progesterone, putrescine, sodium selenite, or triodo-I-thyronine, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

### Preservatives

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include, ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

### Dosing and Administration

The cells and compositions of the present disclosure described above may be administered by any delivery route, systemic delivery, or local delivery, which results in a therapeutically effective outcome.

In some embodiments, thymic cells and effector cells may be co-delivered to the same anatomic location in a subject. In some embodiments, thymic cells and effector cells may be delivered to the different anatomic locations in a subject.

In some embodiments, thymic cells and effector cells may be delivered to the subject at the same time through the same delivery route or through a different delivery route.

In some embodiments, thymic cells may be administered to the subject prior to the administration of effector cells.

In some embodiments, thymic cells may be administered to the subject after the administration of effector cells.

Non-limiting examples of delivery routes include, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions containing cells of the disclosure may be delivered intrathymically (into the thymus).

In some embodiments, compositions containing cells of the disclosure may be surgically placed in the subject. As non-limiting examples, the cells may be surgically placed in the kidney capsule or in the quadriceps muscles in the thigh.

In some embodiments, thymic cells and/or compositions, containing cells of the disclosure may be administered intra hepatically, via intrasplenic injection or via intraportal injection.

thymic cells and/or compositions described herein may be provided to the subject by directly injecting into the marrow of the bone (herein referred to as intraosseous infusion). The bone may be a long bone such as the tibia, fibula, femur, metatarsals, phalanges of the lower limbs, the humerus, radius, ulna, metacarpals, and/or phalanges of the upper limb.

### Parenteral and Injectable Administration

In some embodiments, the cells and compositions described herein may be administered parenterally.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues. Lymph node administration

In certain embodiments, the thymic cells and/or the compositions of the present disclosure may be delivered into the subject for engraftment into the lymph node. In some embodiments, the thymic cells and/or the compositions of the present disclosure may be delivered into the subject in an amount effective to form an ectopic thymus tissue in the lymph node. In certain embodiments, the methods and compositions described herein are used to deliver thymic cells and/or compositions of the disclosure into a lymph node of the subject, allowing the thymic cells to engraft and produce an ectopic thymus in the lymph node. In certain embodiments, the ectopic thymus may restore the thymic function of the subject, e.g., supplements or augments one or more functions that a normal healthy thymus organ can perform. For example, but not by way of limitation, the ectopic thymus may participate in immunomodulation of the body by promoting in T cell growth, development, maturation, and selection.

In certain embodiments, the thymic cells may be delivered into the subject as a liquid suspension.

Non-limiting examples of lymph nodes in which the thymic cells can be delivered include abdominal lymph nodes, celiac lymph nodes, paraaortic lymph nodes, splenic hilar lymph nodes, porta hepatis lymph nodes, left gastric lymph nodes, right gastric lymph nodes, left gastroomental (gastroepiploic) lymph nodes, right gastroomental (gastroepiploic) lymph nodes, retroperitoneal lymph nodes, pyloric lymph nodes (e.g., supra pyloric lymph nodes, sub pyloric lymph nodes, retro pyloric lymph nodes), pancreatic lymph nodes (e.g., superior pancreatic lymph nodes, inferior pancreatic lymph nodes, splenic lineal lymph nodes lymph nodes), splenic lymph nodes, hepatic lymph nodes (e.g., cystic lymph nodes, foraminal lymph nodes, foramen of Winslow), pancreaticoduodenal lymph nodes (e.g., superior pancreaticoduodenal lymph nodes, inferior pancreaticoduodenal lymph nodes), superior mesenteric lymph nodes, ileocolic lymph nodes, prececal lymph nodes, retrocecal lymph nodes, appendicular lymph nodes, mesocolic lymph nodes (e.g., paracolic lymph nodes, left colic lymph nodes, middle colic lymph nodes, right colic lymph nodes, inferior mesenteric lymph nodes, sigmoid lymph nodes, superior rectal lymph nodes), common iliac lymph nodes (e.g., medial common iliac lymph nodes, intermediate common iliac lymph nodes, lateral common iliac lymph nodes, subaortic common iliac lymph nodes, common iliac nodes of promontory), and external iliac lymph nodes (e.g., medial external iliac lymph nodes, intermediate external iliac lymph nodes, lateral external iliac lymph nodes, medial lacunar-femoral lymph nodes, intermediate lacunar-femoral lymph nodes, lateral lacunar-femoral lymph nodes, interiliac external iliac lymph nodes, obturator-external iliac obturatory lymph nodes).

As a non-limiting example, the any of the methods for transplantation of thymic tissue into the lymph node described in International Patent Publication WO2021026195 may be useful in the present disclosure (the contents of which are herein incorporated by reference in its entirety).

### Depot Administration

As described herein, in some embodiments, cells and compositions including pharmaceutical compositions of the present disclosure are formulated in depots for extended release. Generally, specific organs or tissues (“target tissues”) are targeted for administration. In some embodiments, localized release is affected via utilization of a biocompatible device. For example, the biocompatible device may restrict diffusion of the cells in the subject.

In some aspects of the present disclosure, cells, compositions, and pharmaceutical compositions are spatially retained within or proximal to target tissues. Provided are methods of providing pharmaceutical compositions, to target tissues of mammalian subjects by contacting target tissues (which include one or more target cells) with pharmaceutical compositions, under conditions such that they are substantially retained in target tissues, meaning that at least 10, 20, 30, 40, 50, 60, 70, 80, 85, 90, 95, 96, 97, 98, 99, 99.9, 99.99, or greater than 99.99% of the composition is retained in the target tissues. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions administered to subjects are present at a period of time following administration.

### Dose and Regimen

The present disclosure provides methods of administering cells, compositions, and pharmaceutical compositions in accordance with the present disclosure to a subject in need thereof. The pharmaceutical compositions including the cells described may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, and the route of administration.

In certain embodiments, cells described herein, compositions, and pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In some embodiments, a dose of cell, compositions and/or pharmaceutical compositions described herein may be about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10 8, 3×10⁸, or 5×10⁸ cells/kg. In In some embodiments, a dose of cell, compositions and/or pharmaceutical compositions described herein may be at least about 1×10⁶, 1.1×10⁶, 2×10⁶, 3.6×10⁶, 5×10⁵, 1×10⁷, 1.8×10⁷, 2×10⁷, 5×10⁷, 1×10⁸, 2×10⁸, 3×10⁸, or 5×10⁸ cells/kg. In some embodiments, a dose of cell, compositions and/or pharmaceutical compositions described herein may be up to about 1×10⁶, 1.1×10⁶, 2×10⁶, 3.6×10⁶, 5×10⁵, 1×10⁷, 1.8×10⁷, 2×10⁷, 5×10⁷, 1×10⁸, 2×10⁸, 3×10⁸, or 5×10⁸ cells/kg. In some embodiments, a dose of cell, compositions and/or pharmaceutical compositions described herein may be about 1.1×10⁶-1.8×10⁷ cells/kg. In some embodiments, a dose of cell, compositions and/or pharmaceutical compositions described herein may be about 1×10⁷, 2×10⁷, 5×10⁷, 1×10⁸, 2×10⁸, 3×10⁸, 5×10⁸, 1×10⁹, 2×10⁹, or 5×10° cells/kg. In some embodiments, a dose of cell, compositions and/or pharmaceutical compositions described herein may be about 1×10⁷, 2×10⁷, 5×10⁷, 1×10⁸, 2×10⁸, 3×10⁸, 5×10⁸, 1×10⁹, 2×10⁹, or 5×10° cells/kg. In some embodiments, a dose of cell, compositions and/or pharmaceutical compositions described herein may be about 1×10⁷, 2×10⁷, 5×10⁷, 1×10⁸, 2×10⁸, 3×10⁸, 5×10⁸, 1×10⁹, 2×10⁹, or 5×10° cells/kg. In some embodiments, a dose of cell, compositions and/or pharmaceutical compositions described herein may be about 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells/kg. In some embodiments, a dose of cell, compositions and/or pharmaceutical compositions described herein may be about 1-3×10⁷ to 1-3×10⁸ cells/kg.

In certain embodiments, the cell described herein or pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site.

The desired may be delivered at least once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

The desired dosage of the cells of the present disclosure may be administered one time or multiple times. The cells, compositions and pharmaceutical formulations may be administered regularly with a set frequency over a period of time, or continuously as a “continuous flow.” A total daily dose, an amount given or prescribed in 24-hour period, may be administered by any of these methods, or as a combination of these methods.

In some embodiments, delivery of the cell(s) to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The cells of the present disclosure may be used in combination with one or more other therapeutic, prophylactic, research or diagnostic agents, or medical procedures, either sequentially or concurrently. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

For example, the cells of the present disclosure is administered as a biocompatible device that restricts diffusion in the subject to increase bioavailability in the area targeted for treatment. The cell(s) of the present disclosure may also be administered by local delivery.

The term “conditioning regime” refers to a course of therapy that a patient undergoes before stem cell transplantation. For example, before hematopoietic stem cell transplantation, a patient may undergo myeloablative therapy, non-myeloablative therapy or reduced intensity conditioning to prevent rejection of the stem cell transplant even if the stem cell originated from the same patient. The conditioning regime may involve administration of cytotoxic agents. The conditioning regime may also include immunosuppression, antibodies, and irradiation. Other possible conditioning regiments include antibody mediated conditioning (see e.g., Czechowicz et al., 318 (5854) Science 1296-9 (2007); Palchaudari et al., 34 (7) Nature Biotechnology 738-745 (2016); Chhabra et al., 10:8(351) Science Translational Medicine 351ra105 (2016)) and CAR-T mediated conditioning (see, e.g., Arai et al., 26 (5) Molecular Therapy 1181-1197 (2018); each of which is hereby incorporated by reference in its entirety), Conditioning needs to be used create space in the brain for microglia derived from engineered HSCs to migrate into to deliver the protein of interest (recent gene therapy trials for ALD and MLD). The conditioning regimen is also designed to create niche “space” to allow the transplanted cells to have a place in the body to engraft and proliferate. In hematopoietic stem cell transplantation, for example, the conditioning regimen creates niche space in the bone marrow for the transplanted hematopoietic stem cells to engraft into. Without a conditioning regimen the transplanted hematopoietic stem cells cannot engraft. In some embodiments, a subject may be dosed with cells, compositions and/or pharmaceutical formulation of the present disclosure following treatment with a conditional regime.

Use of the cells described in the present disclosure for treatment of a disease, disorder, or condition is also encompassed by the disclosure.

Certain embodiments provide the disease, the disorder, or the condition as selected from cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions.

In certain embodiments, the disease, the disorder, or the condition affects at least one system of the body selected from muscular, skeletal, circulatory, nervous, lymphatic, respiratory endocrine, digestive, excretory, and reproductive systems.

Effector cell: As used herein, an “effector cell” refers to any cell or cell type which, when in contact with or in proximity to a thymic cell, acquires the ability to execute, initiate or propagate a signal or a cell death trigger. “Contact or proximity” refers to spatiotemporal closeness sufficient to enable cell-intrinsic or cell-extrinsic (e.g., cell-to-cell) signaling or other communication or interaction.

Immune tolerance response: As used herein, “immune tolerance response” refers to a class of immune response characterized by reduced or eliminated tendency of the immune system to mount an activated immune response to at least one antigen.

Infectious agent antigen: As used herein, the term “infectious agent antigen” refers to a class of antigens which are derived from infectious disease causing agents or microorganisms. Non limiting examples of infectious agent antigens include viral antigens, bacterial antigens, protozoan antigens, prion antigens, and/or fungal antigens.

Lymphocyte: As used herein, a “lymphocyte” embraces the meanings and uses that a person of ordinary skill in the art would understand the term to embrace, and additionally refers to a type of immune cell originating in the bone marrow that resides in lymphoid tissues or blood. In some embodiments, lymphocytes undergo maturation in the thymus.

Negative: As used herein, the term “negative” (which may be abbreviated as “−”), as used herein with reference to expression of the indicated cell marker, means that the cell does not express the indicated cell marker at a detectable level.

Neoantigen: As used herein, the term “neoantigen” embraces the meanings and uses that a person of ordinary skill in the art would understand the term to embrace, and additionally refers to a class of tumor antigens which arises from tumor-specific mutations in expressed proteins.

Positive: As used herein, the term “positive” (which may be abbreviated as “+”), with reference to expression of the indicated cell marker, means that the cell expresses the indicated cell marker at any detectable level, which may include, for example, expression at a low (but detectable) level as well as expression at a high (hi) level.

Pre-T cell: As used herein, a “pre-T cell” refers to a lymphocyte that is capable of maturing or differentiating into a T cell.

Self-antigen: As used herein, the term “self-antigen” embraces the meanings and uses that a person of ordinary skill in the art would understand the term to embrace, and additionally refers to a class of antigens derived from the one or more cells or cell types of a parent organism that induce an immune response in another organism, but does not induce an immune response in a healthy parent organism from which it was derived.

Thymic origin or lineage: As used herein, “thymic origin or lineage” refers to a cell with one or more phenotypic or genotypic markers associated with a cell derived from the thymus or a cell destined to become a cell of the thymus. As used herein, the thymus may be an embryonic, a fetal or an adult thymus.

Tumor antigen: As used herein, the term “tumor antigen” embraces the meanings and uses that a person of ordinary skill in the art would understand the term to embrace, and additionally refers to a class of antigens which are derived from cancer cells.

Tumor associated antigen (TAA): As used herein “tumor associated antigen” refers to a class of tumor antigens derived from tumor cells that may also be derived from one or more non-tumor cells or cell types.

Tumor specific antigen (TSA): As used herein “tumor specific antigen” refers to a class of tumor antigen characterized in that it is specific to a particular tumor or cancer cell.

Variant: The term “variant” as used in reference to a biomolecule refers to a biomolecule that is related to or derived from a parent molecule. The variant can be, for example, a modified form, a truncated form, a mutated form, a homologous form, or other altered form of the parent molecule. The term variant can be used to describe either polynucleotides or polypeptides.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### Equivalents And Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1. Preparation of Thymic Cells with Alginate Encapsulation

### Expansion of IPSC Line

On day 0, iPSC line (3 to 4 days in suspension; 300-400 microns in diameter) was treated using Accutase to prepare single cells from aggregates. Cells were then resuspended in Stemscale media (Thermo Fisher) containing 1% pen strep and 10 μM Y-27632 and placed on ice for 30 minutes before encapsulation.

Encapsulation of iPSC by Extrusion

Autoclaved alginate solution was prepared with 1.1% alginate, 0.2% gelatin, 10 mM HEPES, and 0.9% NaCl. Cells were resuspended in an alginate solution at a concentration of 200,000 cells/mL. Solutions were loaded into a 1 ml syringe equipped with a 27-gauge needle tip. The syringe was then placed into a syringe pump and extruded into a 100 mM CaCl₂) gelling bath at a rate of 0.25 mL/minute. Alginate capsules were allowed to remain in the bath for an additional 5 minutes. The capsules were then washed with PBS and suspended into Stem Scale media containing 1% pen strep and 10 μM Y-27632. Cells were cultured in this medium for 5 days before differentiation. Differentiation was carried out using 3 mL of each differentiation media per mL of capsules. Complete media changes were done daily.

Differentiation of iPSCs into Definitive Endoderm (DE)

On day 1, cells were washed with PBS and were resuspended in Media A. Media A was prepared to include Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), and NEAA (1:400). On day 2, 1 ml supernatant from each well was replaced with 1 ml freshly prepared Media A.

On day 3, cells were washed with PBS and was resuspended in Media B. Media B was prepared to include Basal Media: DMEM-F12, Activin A (100 ng/mL), LDN193189 (200 nM), PI-103 (25 nM), Insulin-Transferrin-Selenium (ITS-G) (1:1000), KSR (0.05%), PenStrep (1:200), and NEAA (1:400).

On day 4-5, 1 ml supernatant from each well was replaced with 1 ml freshly prepared Media B. Media B was prepared to include Basal Media: DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200), and NEAA (1:400).

Differentiation of Definitive Endoderm (DE) into Anterior Foregut Endoderm (AFE)

24-well plates were coated with Geltrex (1:100) placed in room temperature for 1 hour. On day 6 the supernatant alongside alginate capsules were transferred into 15-ml conical tubes. The alginate capsules were centrifuged at 250 G for 5 min at room temperature and aspirate the supernatant. The alginate capsules were washed with PBS and resuspended in AFE media and cultured from day 6-8. AFE media was prepared to include Basal Media: DMEM-F12, LDN193189 (200 nM), SB431542 (10 μM), FGF8b (50 ng/ml), Ascorbic Acid (10 μM), Pen Strep (1:200), B27 (without RA) (1:200), N2 (1:100), Glutamax (1:100), BME (1:100), and NEAA (1:400).

On day 7, equal amount of freshly prepared AFE media (500 μl) was added per well. On day 8, 50% of supernatant was removed and freshly prepared AFE media (500 μl) added on each well.

### Differentiation of Anterior Foregut Endoderm (AFE) to Ventral Pharyngeal Endoderm (VPE)

On day 9, equal amount of freshly made VPE1 media (500 ul) was replaced gently without disturbing the alginate capsules. VPE1 was prepared to include Basal Media: DMEM-F12, SB431542 (10 μM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2 (1:100) and NEAA (1:400). On day 10, equal amount of freshly prepared VPE1 media (500 μl) was added per well.

Day 12, equal amount of freshly made VPE2 media (500 ul) was replaced gently without disturbing the alginate capsules. VPE2 media was prepared to include Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), N2 (1:100)

On day 13, equal amount of freshly prepared VPE2 media (500 μl) was added per well.

### Differentiation of Ventral Pharyngeal Endoderm (VPE) to Thymic Epithelial Progenitors (TEP)

On day 14, equal amount of freshly made TEP media (500 μl) was replaced gently without disturbing the alginate capsules. The cells were cultured in TEP media from days 14-18. TEP media was prepared to include Basal Media: DMEM-F12, FGF10 (50 ng/ml), BMP4 (50 ng/ml), FGF8b (50 ng/ml), CHIR99021 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2 (1:100), and NEAA (1:400). On day 15, equal amount of freshly prepared TEP media (500 ul) was added per well. On days 16-18, 50% of supernatant was removed and freshly prepared TEP media (500 ul) was added per well.

On days 19-22, cells were cultured in TEC media. On day 19, equal amount of freshly made TEC media (500 μl) was replaced gently without disturbing the alginate capsules. TEC media was prepared to include Basal Media: DMEM-F12, FGF10 (50 ng/ml), IL-22 (20 ng/ml), FGF7/KGF (50 ng/ml), RANKL/TRANCE (50 ng/ml), BMP4 (50 ng/ml), FGF8b (50 ng/ml), CHIR99021 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2 (1:100), NEAA (1:400), Glutamax (100×), and BME (100×). On day 20, equal amount of freshly prepared TEC media (500 ul) along with Plurisin #1 (20 μM) was added (Groups-Untreated and Treated). On day 21, equal amount of freshly made TEC media (500 ul) was replaced gently without disturbing the alginate capsules.

### Removal of Alginate to Harvest Differentiated Cells

Alginate capsules were disassociated using a de-encapsulation solution containing 55 mM sodium citrate, 10 mM HEPES, and 0.9% NaCl. Capsules were suspended in the de-encapsulation solution for 10 minutes while shaking at 100 rpm. The aggregates were collected by centrifugation (1100 rpm for 4 minutes) and then treated with Accutase to dissociate into single cells. The single cells were collected by centrifugation (1100 rpm for 4 minutes) and used for further testing.

### Example 2. Characterization of TEPs Derived by Differentiation of Alginate Encapsulated iPS Cells in 3D Suspension

Single cell iPS cells were encapsulated in low density alginate microbeads and cultured in 3D suspension. By day 4, aggregates of different sizes were observed, indicating that iPS cells can proliferate within alginate and form colonies. Differentiation was induced as described in Example 1 by addition of culture medium for DE, AFE, VPE, TPE and TEC. By the TEC stage (day 15-16), aggregates were bigger (around 600 microns) and fewer in number.

The expression of TEP markers was compared between iPSC aggregates that had been differentiated in alginate capsules using 3D suspension culture and iPS differentiated in 2D conditions and also after culture in 2D and subsequent freeze and thaw. FOXN1 expression was observed in alginate TEC at a level comparable to that in two dimensional culture conditions prior to and after being frozen and rethawed into aggregate suspension culture in TEC medium for 5 days. FOXN1 was expressed at highest level after freeze- and thaw. The expression of HOXA3 and DLL4 was higher in alginate TEC and only the alginate TEC showed expression of PAX1. The data showed that iPS cells can be differentiated in 3D aggregates all the way from iPS to TEC (FIG. 1).

### Example 3. Differentiation of Chitosan-Coated, Alginate-Encapsulated iPSC Aggregates into Thymic Cells Using 3D Suspension Culture

Expansion of IPSC line (Day −4 or Day −3)

An iPSC line was thawed and grown in Stemscale media (Thermo Fisher) in six well plates containing Pen Strep (1:100) and 10 μM Y-27632 in an incubator at 37 C, 5% CO₂, on a platform shaking at 70 rotations per minute (rpm). Once the aggregates reached an average diameter of 300-400 μm (3-4 days), the expanded iPSCs were placed on ice for 30 minutes before encapsulation.

### Preparation of Solutions for Chitosan-Alginate Encapsulation

A buffered saline solution was prepared which contained 10 mM HEPES and 0.9% NaCl. An encapsulation solution was prepared which contained 10 mM HEPES, 0.9% NaCl, 0.2% Gelatin Type A, and 1.1% low viscosity sodium alginate. This was mixed at maximum stirring speed for at least 2 hours. CaCO₃ was added to bring the solution to a concentration of 24 mM, and the entire solution was autoclaved.

Sterile glacial acetic acid was filtered using a 22 um filter and added to sterile mineral oil to create a 0.4% glacial acetic acid and mineral oil mixture which was then stirred at maximum speed for 1-2 minutes and autoclaved.

A 1.66% low molecular weight Chitosan coating solution was prepared using 0.5 M HCl. It was allowed to dissolve for two hours while stirring at 1000 rpm, and then autoclaved.

A de-encapsulation solution was prepared which contained 10 mM HEPES, 95 mM NaCl, and 55 mM sodium citrate.

Encapsulation of hiPSC Aggregates in Chitosan-Coated Alginate Microbeads Via Emulsification

Approximately 200,000 hiPS cells were resuspended in 10 mL of encapsulation solution and vortexed for 20-30 seconds to create a homogenous mixture. All 10 mL of this homogenized mixture was slowly added to 20 mL of mineral oil and stirred at 500 rpm for 12 minutes to create an emulsion. Under continuous stirring, 10 mL of a 0.4% glacial acetic acid and mineral oil mixture was added to the emulsion and stirring was continued for 8 minutes. After completion of gelling, 40 mL of DMEM/F12 media was added and stirred for 1 minute to neutralize the pH. The emulsion was separated into two 50 ml conical tubes and centrifuged at 700×g for 4 minutes to separate the oil and collect the capsules. The oil layer was removed and a vacuum trap was used to remove as much media as possible. Alginate beads were suctioned out of each conical tube then resuspended in 40 mL of buffered saline. Separately, 9 mL of chitosan coating solution was combined with 21 mL of buffered saline solution to create 30 mL of chitosan-saline solution. 10 mL of this chitosan-saline solution was added to each conical tube in order to resuspend the hiPS-containing alginate microbeads. The resuspended alginate microbeads were slowly added into the remaining chitosan+buffered saline solution and stirred at 500 rpm for 1 minute. The solution was then strained using 40 um cell strainers. The strained chitosan-coated alginate microbeads, which contained hiPSC aggregates, were washed with 10 mL of buffered saline and placed into shaker flasks with 24 mL of StemScale media supplemented with 1% PenStrep and 2 mM Y-27632. The shaker flasks were put on a shaker platform at 70 rpm and incubated for 3-4 days at 37° C. and 5% CO₂ before differentiation begins.

### Preparation of Cell Culture Media

Media A was prepared with DMEM-F12 as the basal media and the following reagents: Activin A (100 ng/mL), CHIR99021 (2 uM), Insulin-Transferrin-Selenium (ITS-G) (1:1000), KSR (0.05%), PenStrep (1:200), PI-103 (25 nM), NEAA (1:400).

Media B was prepared with DMEM-F12 as the basal media and the following reagents: Activin A (100 ng/mL), LDN 193189 (200 nM), Insulin-Transferrin-Selenium (ITS-G) (1:1000), KSR (0.05%), PenStrep (1:200), PI-103 (25 nM), NEAA (1:400).

AFE Media was prepared with DMEM-F12 as the basal media and the following reagents: LDN 193189 (200 nM), SB431542 (10 uM), FGF8b (50 ng/ml), Ascorbic Acid (10 uM), PenStrep (1:200), B27 (without retinoic acid) (1:200), N2 (1:100), BME (1:100), KSR (0.05%), NEAA (1:400), Glutamax (1:100).

VPE1 Media was prepared with DMEM-F12 as the basal media and the following reagents: SB431542 (10 uM), FGF8b (50 ng/ml), Retinoic Acid (0.1 nM), Ascorbic Acid (10 uM), Insulin-Transferrin-Selenium (ITS-G) (1:1000), KSR (0.05%), PenStrep (1:200), B27 (without vitamin A) (1:200), Glutamax (1:100), BME (1:100), N2 (1:100), NEAA (1:400).

VPE2 Media was prepared with DMEM-F12 as the basal media and the following reagents: Noggin (50 ng/mL), CHIR99021 (2 uM), FGF8b (50 ng/mL), Retinoic Acid (0.1 nM), Ascorbic Acid (10 uM), Insulin-Transferrin-Selenium (ITS-G) (1:1000), KSR (0.05%), PenStrep (1:200), NEAA (1:400), B27 (without vitamin A) (1:200), Glutamax (1:100), BMB (1:100) and N2 (1:100).

TEP Media was prepared with DMEM-F12 as the basal media and the following reagents: FGF10 (50 ng/mL), BMP4 (50 ng/mL), FGF8b (50 ng/mL), CHIR99021 (2 uM), Ascorbic Acid (10 uM), Insulin-Transferrin-Selenium (ITS-G) (1:1000), KSR (0.05%), PenStrep (1:200), and NEAA (1:400).

TEC Media was prepared with DMEM-F12 as the basal media and the following reagents: IL-22 (20 ng/mL), FGF10 (50 ng/mL), FGF7/KGF (50 ng/mL), RANKL/TRANCE (50 ng/mL), BMP4 (50 ng/ml), FGF8b (50 ng/mL), CHIR99021 (2 uM), Ascorbic Acid (10 uM) and Insulin-Transferrin-Selenium (ITS-G) (1:1000).

TEC Freeze/Thaw (FT) Media was prepared with DMEM-F12 as the basal media and the following reagents: IL-22 (20 ng/mL), FGF10 (50 ng/mL), FGF7/KGF (50 ng/ml), RANKL/TRANCE (50 ng/mL), BMP4 (50 ng/mL), FGF8b (50 ng/mL), CHIR99021 (3 uM), Ascorbic Acid (10 uM), Insulin-Transferrin-Selenium (ITS-G) (1:1000), KSR (0.0025%), PenStrep (1:200), B27 without vitamin A (1:200), Glutamax (1:100), BME (1:100), N2 (1:100) and NEAA (1:400).

Differentiation of Chitosan-Coated, Alginate Encapsulated hiPSCs into Definitive Endoderm (DE)

On Day 0 the hiPSCs were disaggregated using Accutase. 3-5 million single iPS cells were placed in suspension in 6-well ultra-low adhesion plates along with Media A and ROCK inhibitor Y-27632 and incubated at 37° C. and 5% CO₂ at 70 rpm. The incubator conditions remained constant during the iPS differentiation steps described herein, and the plated cells were returned to the incubator after each differentiation step, unless expressly stated otherwise.

On Day 1, 1 mL of supernatant was removed from each well of each 6-well plate and spun down. The pelleted cells were washed with PBS and resuspended in 1 mL of pre-warmed Media A and Y-27632 before being added back to the sixth well of each plate (well #6).

On Day 2, approximately 1.8 mL of the supernatant from each well was removed and 2 mL of pre-warmed Media A (without Y-27632) was added to each well. The supernatant was spun down and the pelleted cells were washed with PBS and resuspended in 2 mL of Media A (without Y-27632) before being added to the sixth well of each plate.

On Day 3, the cells along with the supernatant were collected in a 50 mL conical tube and allowed to settle. Remaining aggregates were collected by adding 2 mL of PBS to each well and transferring the remaining cells to a new conical tube. 1 mL of fresh Media B was added to each well and the plates were returned to the incubator. The supernatant (Media A) was aspirated from the settled cells in each conical tube. 20 mL of phospho-buffered saline (PBS) was added to each tube, and the tubes were gently vortexed and spun down at 1100 rpm for 4 minutes. The cells were resuspended in the required amount of Media B (based on the number of plates) and distributed equally into the wells of each plate.

On Days 4 and 5, 1 mL supernatant was removed from each well replaced with 1 mL of pre-warmed Media B. The collected supernatant was spun down and residual cells were added to the sixth well of each plate (well #6).

Differentiation of DE Cells into Anterior Foregut Endoderm (AFE) Cells

On Day 6, the aggregates were collected along with supernatant and allowed to settle. 1-2 mL of PBS were added to each well and the remaining aggregates were collected in a new tube. 1 mL of freshly prepared AFE media was added to each well and the plates were returned to the incubator. The supernatant was aspirated from the settled cells and 20 mL PBS was added before spinning the cells down at 1100 rpm for 4 minutes. The pelleted cells were resuspended in the required amount of AFE media and distributed equally in the six-well plates.

On Days 7-8, 1 mL of supernatant from each well was replaced with 1 mL freshly prepared AFE media. The 1 mL of supernatant from each well was spun down and removed, and the residual cells were resuspended in AFE media and added back to the last well.

### Differentiation of Anterior Foregut Endoderm (AFE) Cells to Ventral Pharyngeal Endoderm (VPE) Cells

On the start of day 9, the aggregates were collected with the supernatant in 50 mL conical tubes and allowed to settle. 1-2 mL of PBS were added to each well and the remaining aggregates were collected in a new tube. 1 mL of freshly prepared VPE1 media was added to each well and the plates were returned to the incubator. The supernatant was aspirated from the settled cells and 20 mL PBS was added before spinning the cells down at 1100 rpm for 4 minutes. The pelleted cells were resuspended in the required amount of VPE1 media and distributed equally in the six-well plates.

On Day 10, 1 mL of the supernatant was collected from each well and replaced with 1 mL of pre-warmed VPE1 media. The collected supernatant was spun down and removed, and the residual cells were resuspended in the required amount of VPE1 media and added to the sixth well of each plate.

On Day 11, the aggregates were collected with the supernatant in 50 mL conical tubes and allowed to settle. 1-2 mL of PBS were added to each well and the remaining aggregates were collected in a new tube. 1 mL of freshly prepared VPE2 media was added to each well and the plates were returned to the incubator. The supernatant was aspirated from the settled cells and 20 mL PBS was added before spinning the cells down at 1100 rpm for 4 minutes. The pelleted cells were resuspended in the required amount of VPE2 media and distributed equally in the six-well plates.

On Day 12, 1 mL of supernatant was collected from each well and replaced with 1 mL of pre-warmed VPE2 media. The collected supernatant was spun down and removed, and the residual cells were resuspended in the required amount of VPE2 media and added back to the sixth well of each plate.

### Differentiation of Ventral Pharyngeal Endoderm (VPE) to Thymic Epithelial Progenitors (TEP)

On Day 13, the aggregates along with the supernatant were collected in 50 mL conical tubes and allowed to settle. 1-2 mL of PBS were added to each well and the remaining aggregates were collected in a new tube. 1 mL of freshly prepared TEP media was added to each well and the plates were returned to the incubator. The supernatant was aspirated from the settled cells and 20 mL PBS was added before spinning the cells down at 1100 rpm for 4 minutes. The pelleted cells were resuspended in the required amount of TEP media and distributed equally in the six-well plates.

On Days 14, 15 and 16, 1 mL of the supernatant was collected from each well and replaced with 1 mL of pre-warmed TEP media. The collected supernatant was spun down and removed, and the residual cells were resuspended in the required amount of TEP media and added to the sixth well of each plate.

On day 17, about 1.8 mL of the supernatant from each well was collected and 2 mL of pre-warmed TEP media was added in its place. The collected supernatant was spun down and removed, and the residual cells were resuspended in TEP media and added back to the last well (well #6). In cases of large numbers of aggregates, the residual cells were added back to 2 or more wells.

On day 18, 1 mL of supernatant from each well was collected and 1 mL of pre-warmed TEP media was added in its place. The collected supernatant was spun down and removed, and the residual cells were resuspended in pre-warmed TEP media and added back to the last well (well #6). This step was repeated on day 19, day 20, day 21 and day 22.

### Removal of Chitosan-Alginate to Harvest and Freeze Differentiated Cells

On day 23, the chitosan-coated alginate capsules were de-encapsulated by suspending them for 10 minutes while shaking at 100 rpm in the de-encapsulation solution (55 mM sodium citrate, 10 mM HEPES, and 0.9% NaCl). The TEP aggregates were collected and the plate was washed with DPBS to collect any remaining TEP aggregates. After letting the TEP aggregates settle, the supernatant was aspirated. The TEP aggregates were treated with Accutase to dissociate into single cells, and a sample of 0.5 to 1.0 mL was removed for cell counting and qRT-PCR.

1-2 mL of fresh TEP media and 10 mL of pre-warmed DMEM-F12 media were added to the cells, and they were spun down at 1110 rpm for 4 minutes. 10 mL of fresh TEP media was added, and the TEP cells were counted using a Cell Counter. The remaining cells were spun down and the supernatant was removed and replaced with cryopreservation media 1 (500 uL per cryopreservation vial). Cryopreservation media 2 was added dropwise to the cells (500 uL per vial) 6-10 million cells were included in each cryopreservation vial and the vials were frozen in liquid nitrogen and stored at −80° C.

### Differentiation of Thymic Epithelial Progenitors to Thymic Epithelial Ells (TEP to TEC)

On day A, the TEPs were thawed and 1 mL of TEC Freeze/Thaw (FT) Media was added to each vial. The thawed cells were transferred to a conical tube and spun down at 1110 rpm for 4 minutes. The supernatant was removed and 6 mL of TEC FT media (with Y27632 at 1:1000) was added to each tube to resuspend the TEPs. TEC FT media with Y27632 at 1:1000 was also added to the wells of each 6-well plate (1 mL per well), and the 1 mL of resuspended cells was added into each well. The plates were placed into the incubator (shaking at 70 rpm, 37° C., 5% CO₂).

24 hours later, on day B, 1 mL of supernatant was removed from each well of each plate and 1 mL of pre-warmed TEC FT media was added to each well. The supernatant was spun down and aspirated, and the residual cells were resuspended in TEC FT media and added back to the last well (well #6) of each plate. These steps were repeated on each of the next three days: (days C, D and E).

On Day F, 1 mL of supernatant was removed from the well of each plate and 1 mL of pre-warmed TEC FT media with PluriSIn (20 μM) was added to each well. The supernatant was spun down and aspirated, and the residual cells were resuspended in TEC FT media with PluriSin (20 uM) and added back to the last well (well #6).

On Day G, the TEC aggregates were collected and washed with DPBS twice. The supernatant was then aspirated and the cells were resuspended in 10 mL of DPBS. Four separate (0.5 to 1 mL) samples were collected and used for cell counting, flow cytometry, qRT-PCR and CFU analyses. For qPCR analyses, the DPBS media was removed from the corresponding 0.5-1.0 mL sample and 200-400 uL RLT buffer was added. The TEC cells in the qPCR sample were lysed and stored at −80° C. for RNA extraction. For the flow cytometry analyses, the TEC aggregates in the 0.5 to 1.0 mL sample were washed with DPBS once and spun down at 1100 rpm for 4 minutes. The supernatant was aspirated and 1-2 mL of Accutase was added to each tube. After a 4 minute incubation, the TEC aggregates were dissociated, washed with 1 mL of DPBS then spun down. The Accutase/supernatant was aspirated and DPBS was added to the cells before they were put on ice for staining.

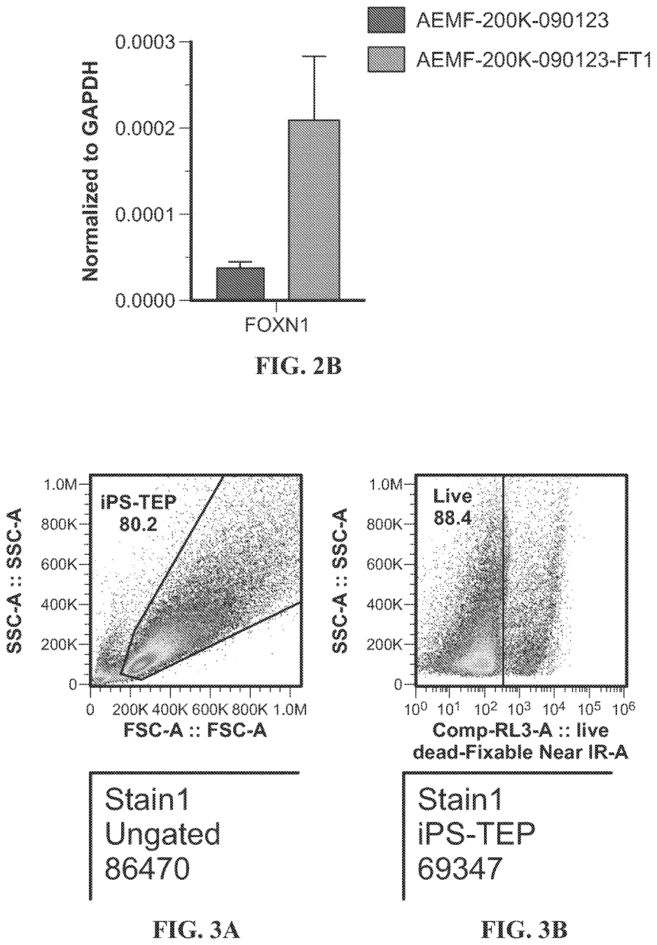

A schematic illustrating the process for differentiation of chitosan-coated alginate encapsulated hiPSC aggregates using 3D suspension culture is provided in FIG. 2A. (“3D Differentiation from Aggregates in Alginate”). The typical level of FOXN1 expression by these TECs at Day G (after having been frozen, thawed and grown for 7 days in TEC FT media), as measured by qRT-PCR normalized to GADPH at a cell concentration of 200,000 TEC cells/mL, is provided in the right-hand column shown in FIG. 2B (0.0002). This level is approximately five-fold higher than the FOXN1 expression level measured at day 23, before the TEC FT media was added to the cells and before they were frozen in liquid nitrogen and stored at −80° C.

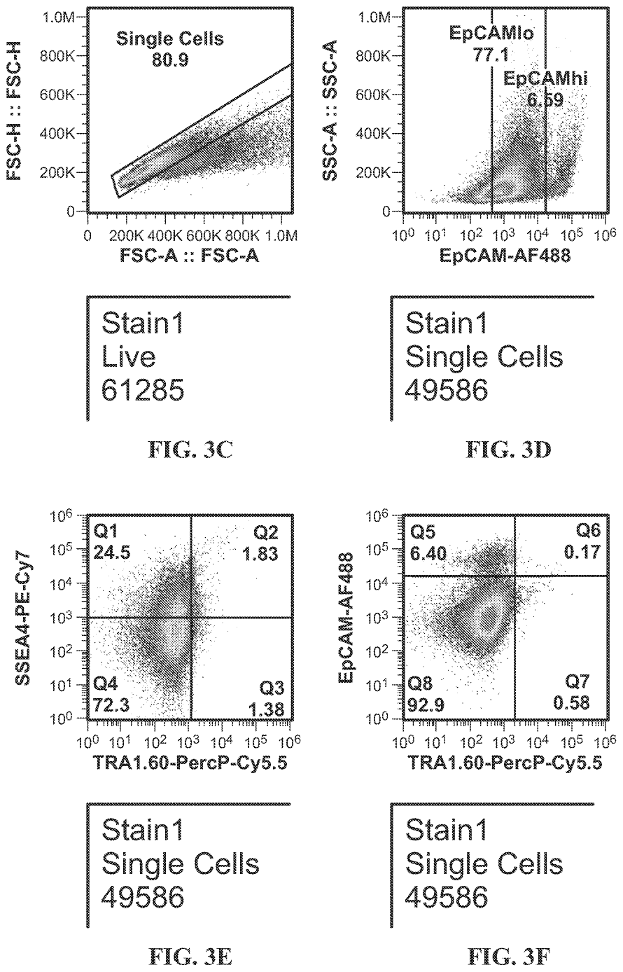

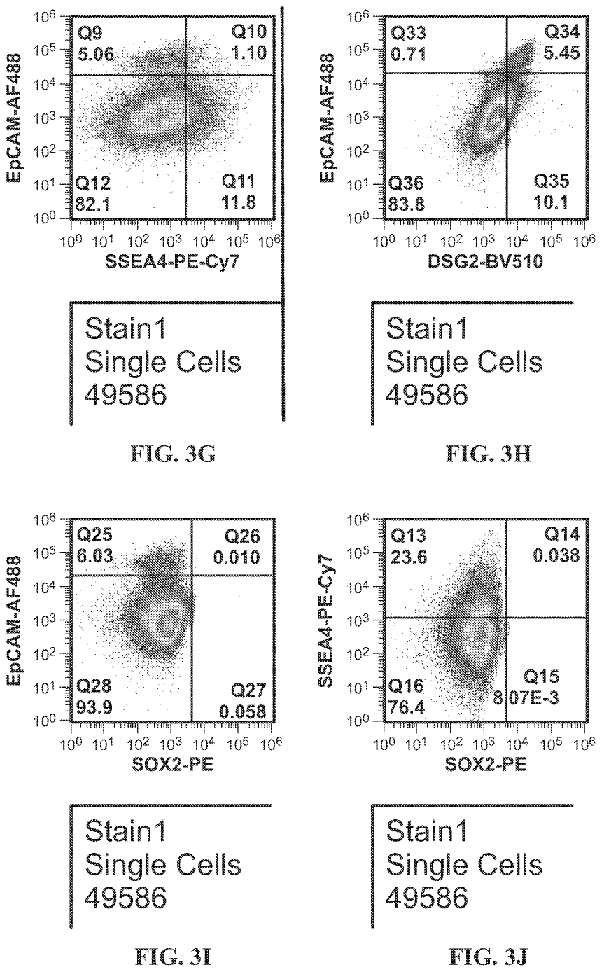

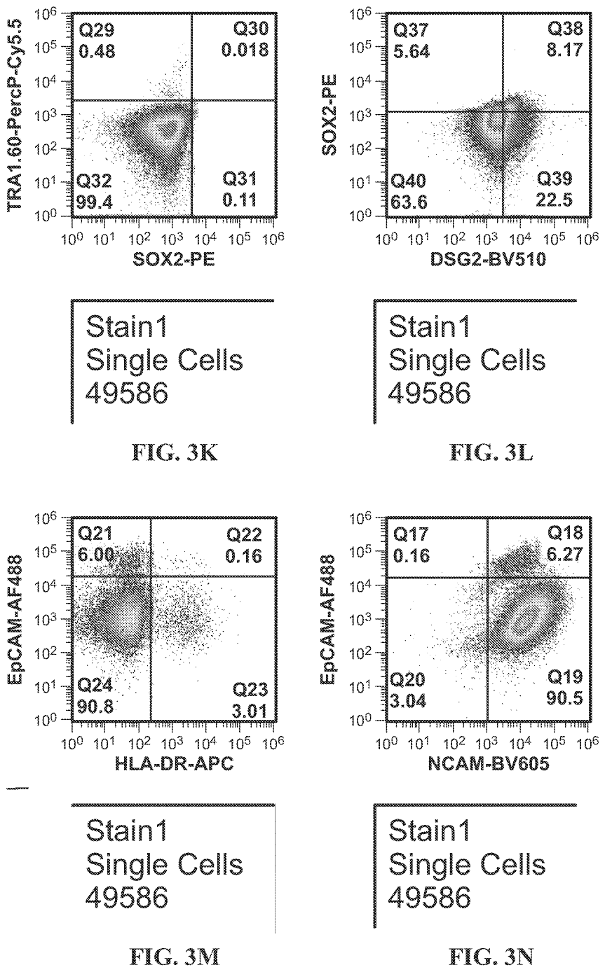

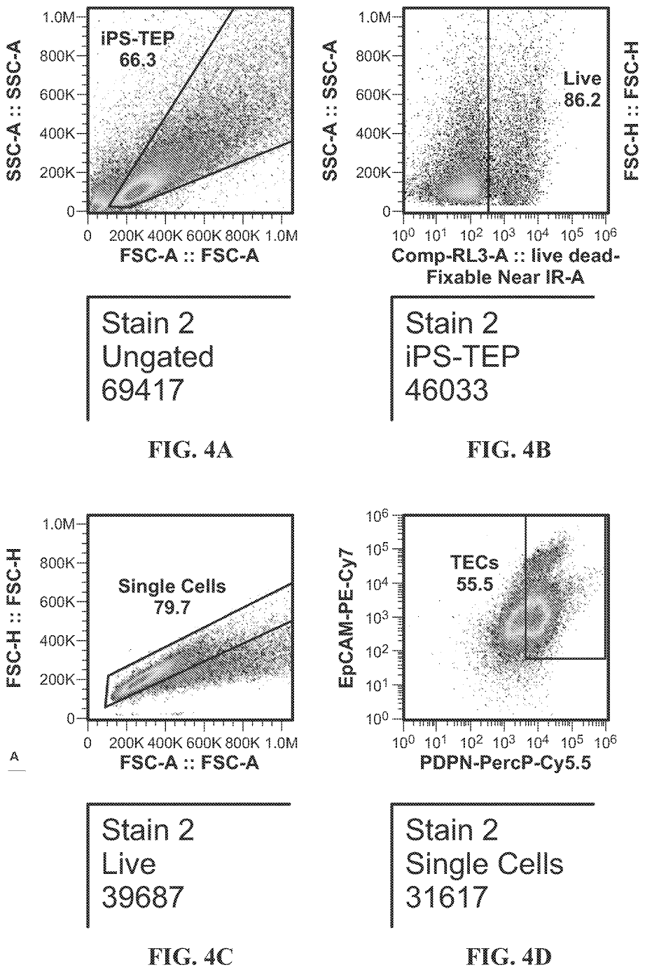

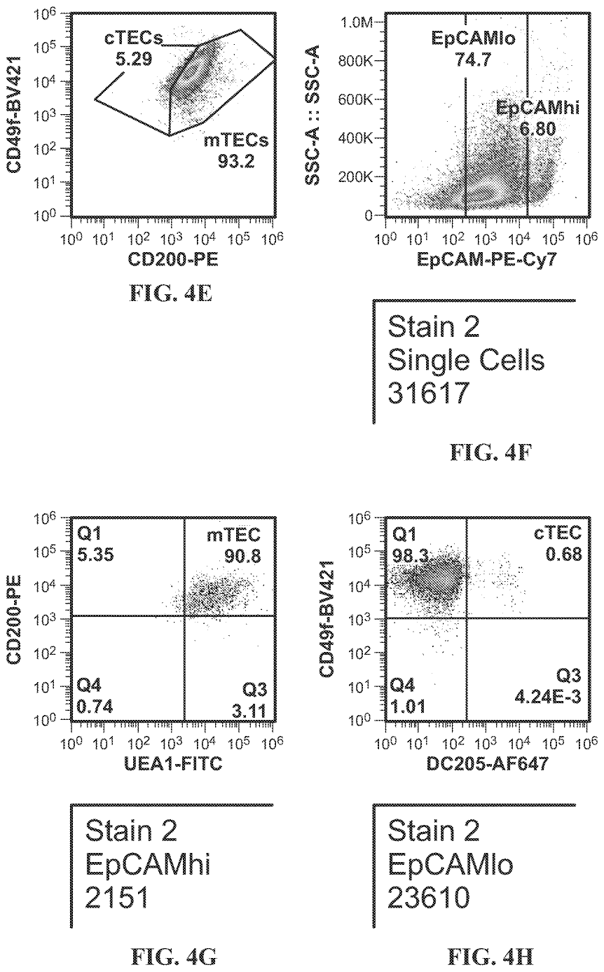

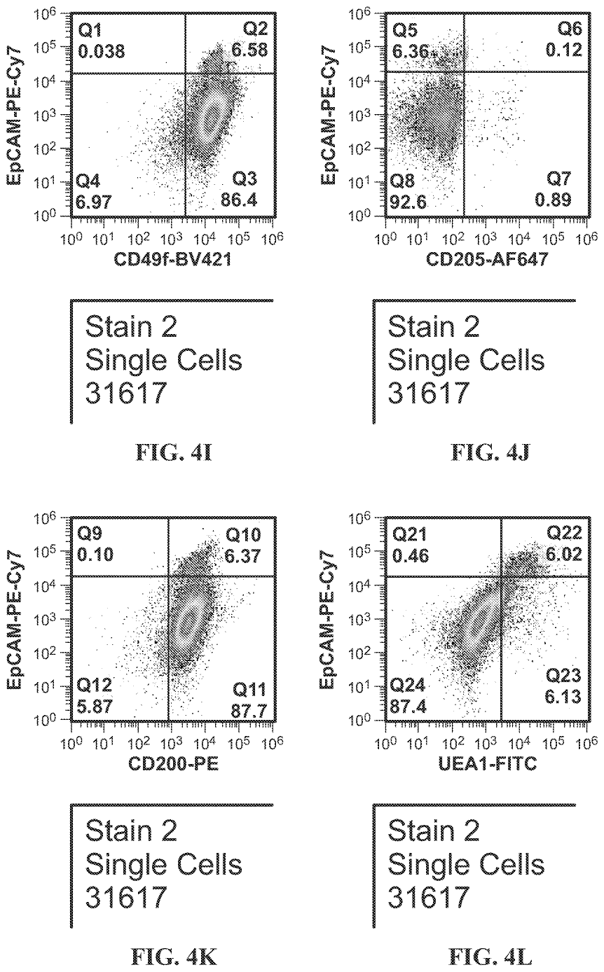

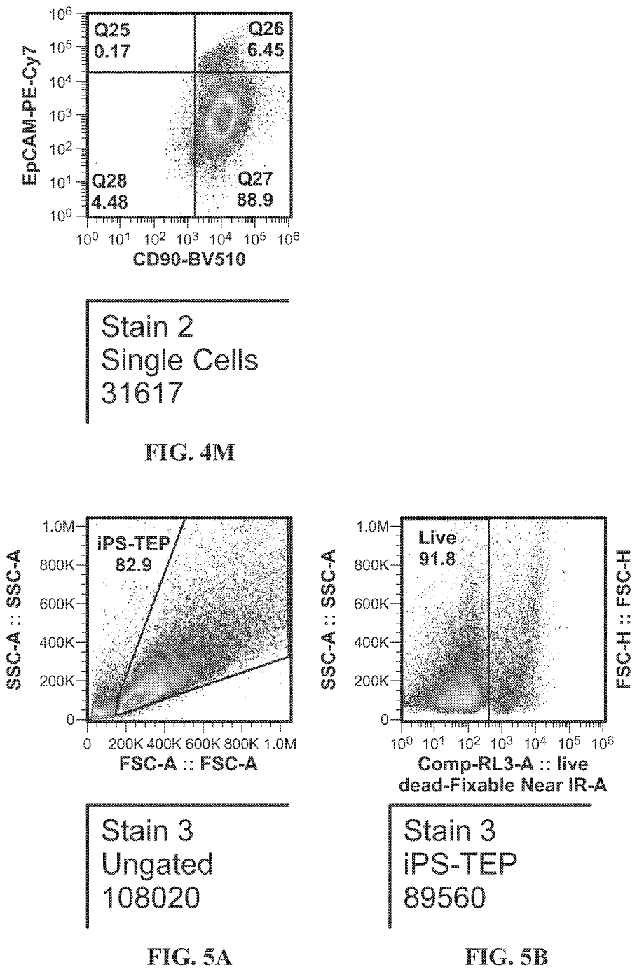

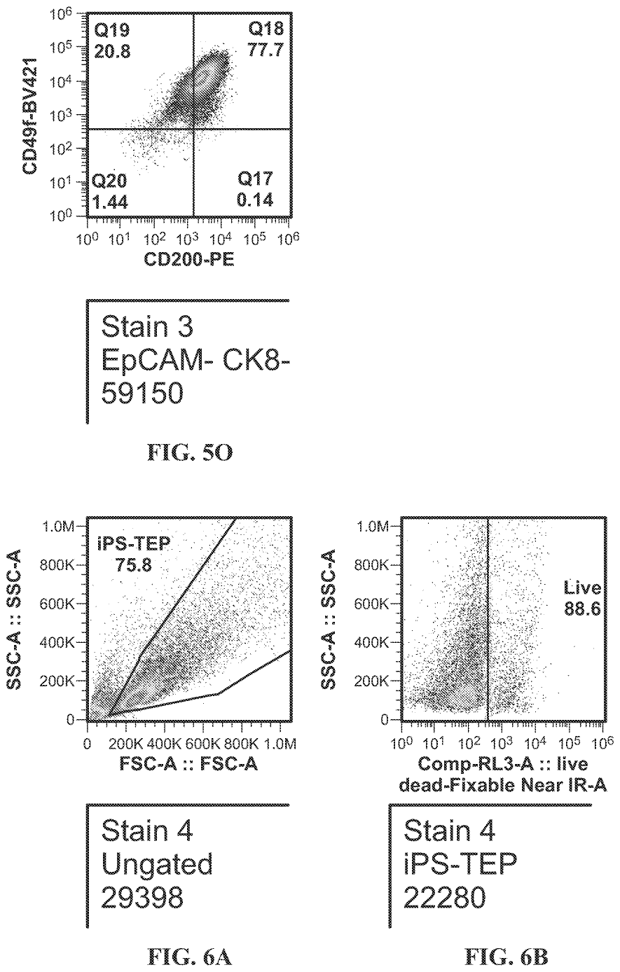

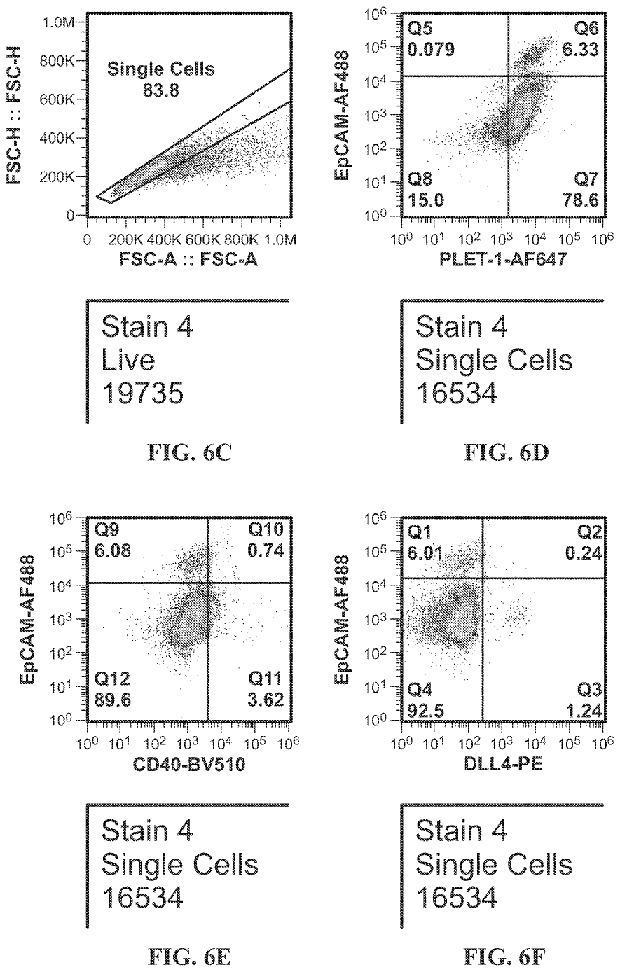

Flow cytometry results for the TEC aggregates are presented in FIG. 3, FIG. 4, FIG. 5 and FIG. 6. Three replicates were performed (Stain 1, FIG. 3A to 3N; Stain 2, FIG. 4A to 4M; Stain 3, FIG. 5A to 5O; Stain 4, FIG. 6A to 6F).

The expression levels for the following genes were assessed for the TEC cells on Day 23, prior to freezing (leftmost columns represent the sample entitled “AEMF-200K-090123 TEC”): Oct4, NANOG, HOXA3, TBX1, EYA1, EPCAM, PAX9, PAX1, FOXN1, DLL4, HLA-DRA, AIRE, CK5, CK8, FEZf2, CLDN3, CLDN4, PRSS16, LY75 and JAG2.

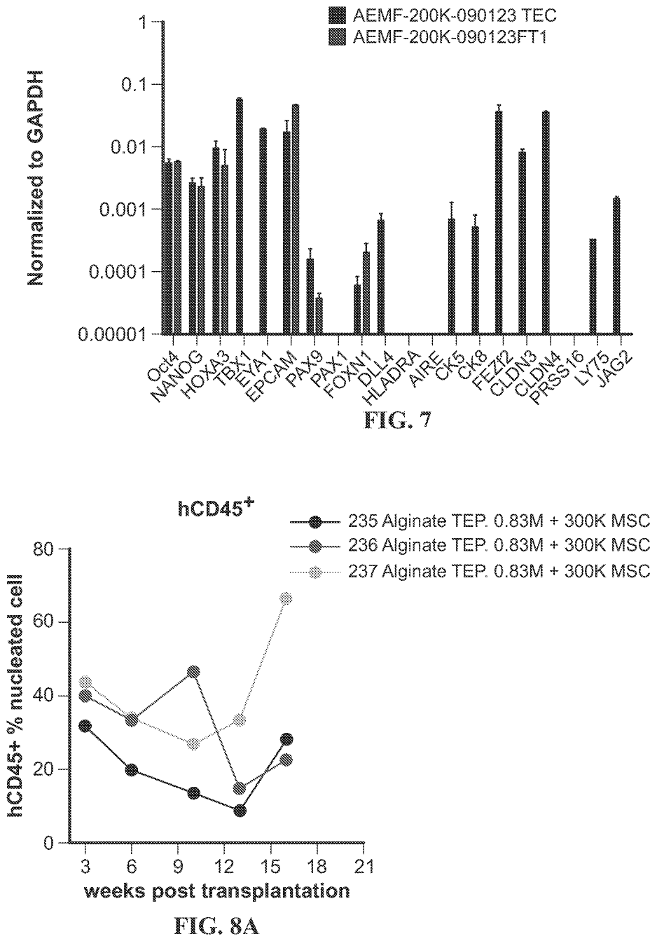

The expression levels for the following genes were assessed on Day G, (after the TEC cells that were frozen on Day 23 were thawed and grown for 7 days in TEC FT media); these results are shown in the columns on the right in FIG. 7: Oct4, NANOG, HOXA3, EPCAM, PAX9 and FOXN1 (“AEMF-200K-090123-FT1”). These results demonstrate the differentiated TECs display thymopoietic capacity for supporting T-cell development in vitro,

### Example 4. Intramuscular Transplantation of Differentiated TEPs Supports Human Thymopoiesis in a Humanized Animal Model

Human iPSC cells encapsulated in alginate and coated with chitosan were grown in 3D suspension culture and differentiated into TEP cells as described in Example 4 (prior to the freeze/thaw step). Approximately 0.83 million TEP cells plus 300K mesenchymal stem cells were injected intramuscularly into humanized NOD/SCID/IL2Rg^{Null} (NSG) MHC-I/II double knockout mice (Jackson Laboratories) (n=3, identified by the numbers 235, 236 and 237). The humanization was accomplished as follows: mice were sub-lethally irradiated, followed by injection of human umbilical cord-blood derived CD34+ hematopocitic stem cell progenitors; 2 weeks later, the 0.83 million TEP cells were injected intramuscularly into the quadricep each mouse (together with 300K mesenchymal stem cells). Humanized NSG mice have inefficient human T cell development due to residual thymic tissue. To create an athymic background in the NSG mice, mice can be thymectomized, but this step was avoided by using an NSG variant strain in which both the MHC Class I and MHC Class II genes have been knocked out. Since MHC is required for thymic function, the double knockout strain creates a functionally athymic background, and the NSG mouse does not produce FOXN1.

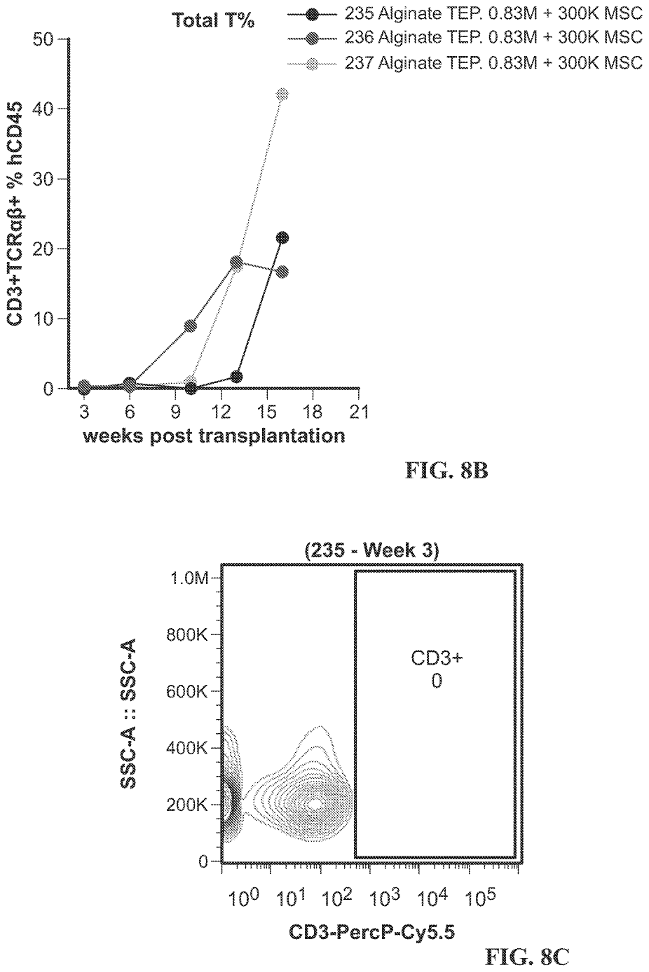

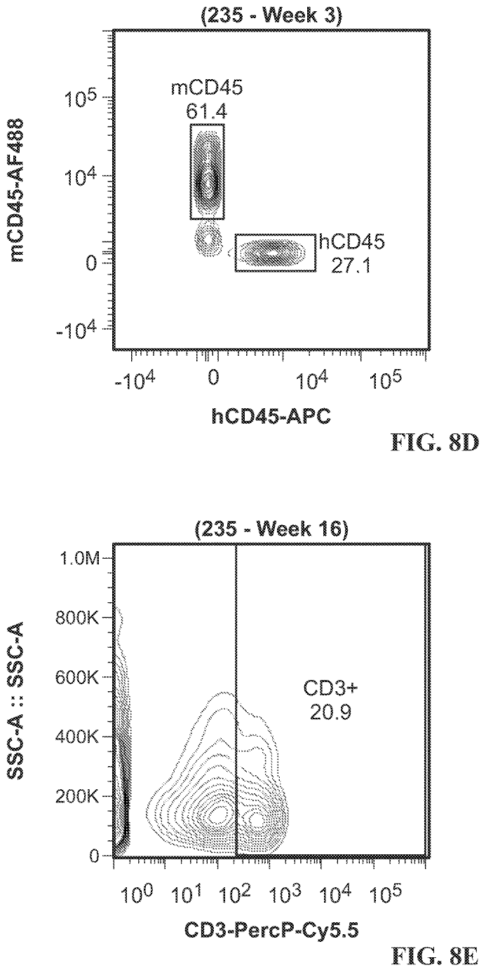

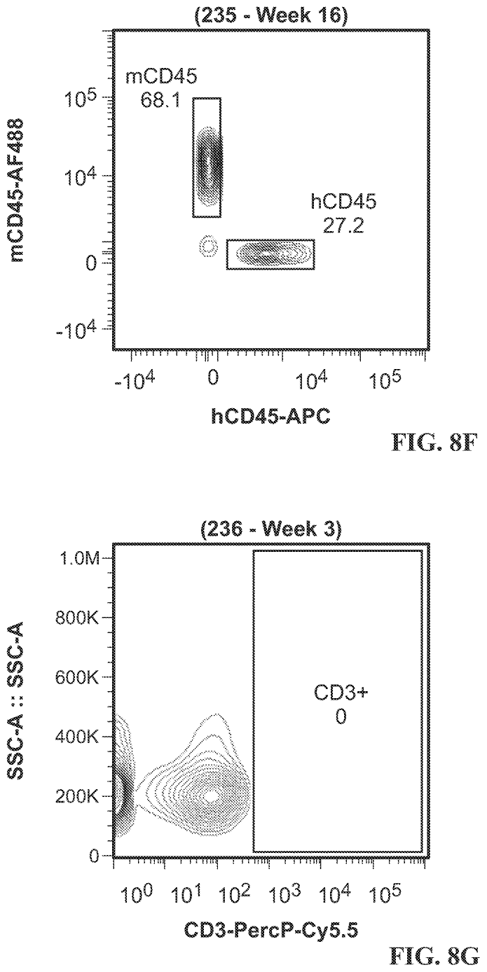

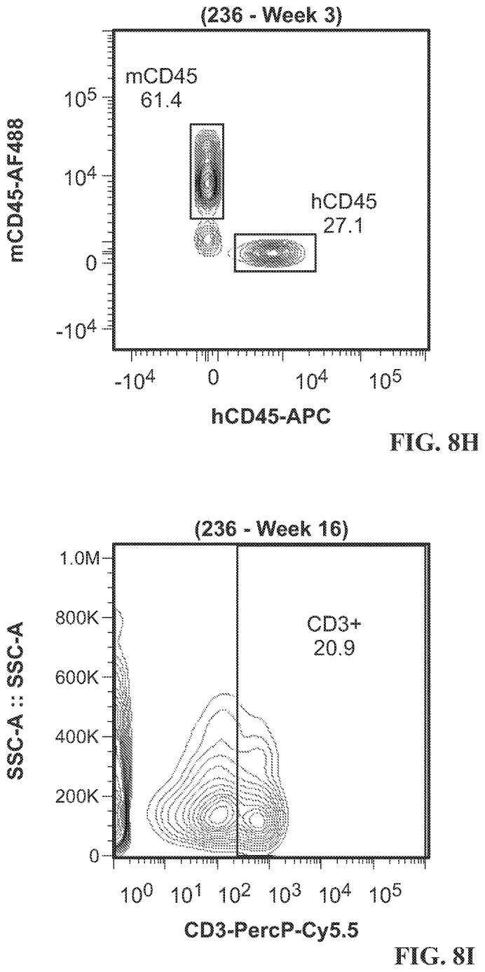

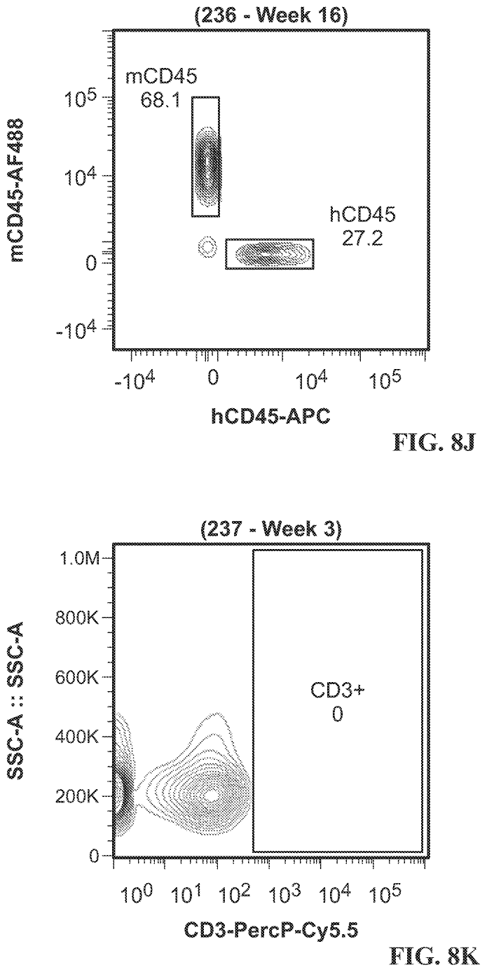

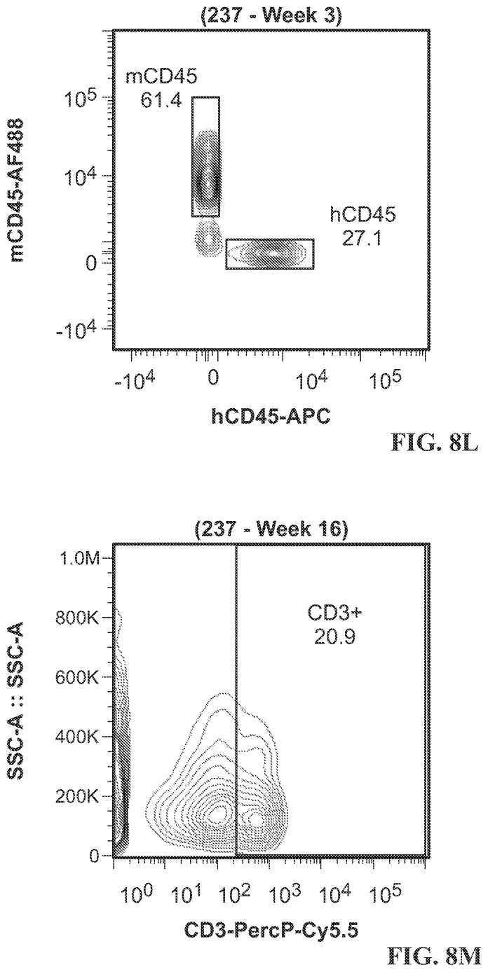

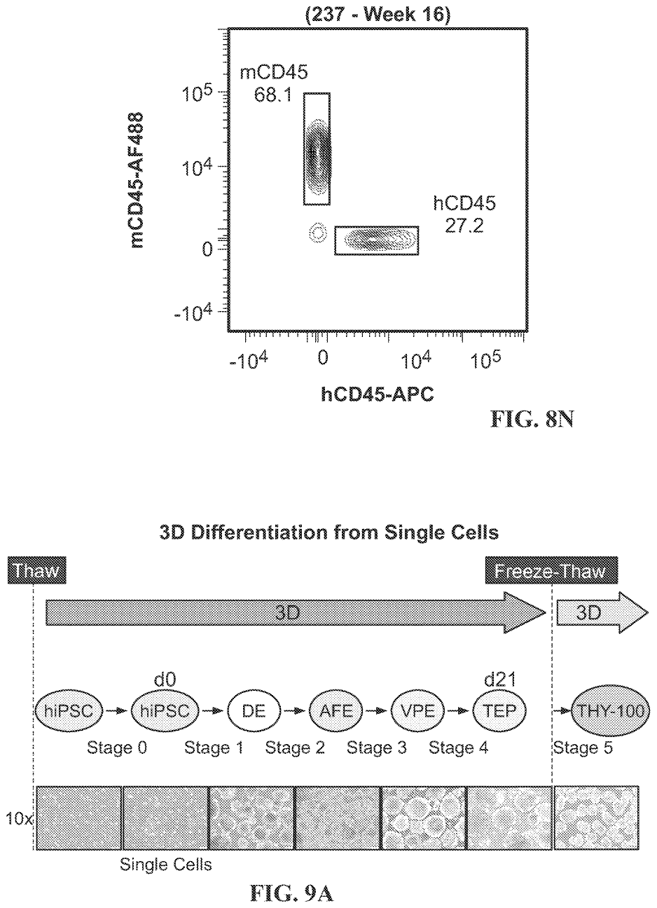

The efficiency of thymopoeisis and generation of T cells were assessed every three weeks via flow cytometry in order to track the emergence of T cells and their immunophenotypes. Markers for human hematopoietic cells (hCD45) and human T cells, such CD3 and TCRαβ, were observed, as shown in FIG. 8A to FIG. 8N. At week 3, none of the mice expressed any CD3+ (FIG. 8G, 8I, 8K). By week 16, CD3 expressing cells Within 13 weeks of the TEP intramuscular injection, human CD3+TCRαβ+ T cells ranged from 2% to 18% of total human blood (CD45+) cells, and by 16 weeks, the range was 15% to 43%. Importantly, no sham transplanted mice (negative control—not shown) developed detectable T cells in their blood, verifying that the mice were effectively athymic and thus an excellent model for testing the thymopoeitic capabilities of iPS-TEPs. These results demonstrated that human iPS-TEPs can efficiently induce the development of human T cells in vivo.

### Example 5. Differentiation of Non-Encapsulated Single hiPS Cells Into Thymic Cells Using 3D Suspension Culture

### Preparation of Cell Culture Media

Media A was prepared with DMEM-F12 as the basal media and the following reagents: Activin A (100 ng/mL), CHIR99021 (2 uM), Insulin-Transferrin-Selenium (ITS-G) (1:1000), KSR (0.05%), PenStrep (1:200), PI-103 (25 nM), NEAA (1:400) and B27 without vitamin A (1:400).

Media B was prepared with DMEM-F12 as the basal media and the following reagents: Activin A (100 ng/mL), LDN 193189 (200 nM), Insulin-Transferrin-Selenium (ITS-G) (1:1000), KSR (0.05%), PenStrep (1:200), PI-103 (25 nM), NEAA (1:400) and B27 without vitamin A (1:400).

AFE Media was prepared with DMEM-F12 as the basal media and the following reagents: LDN 193189 (200 nM), SB431542 (10 uM), FGF8b (50 ng/ml), Ascorbic Acid (10 uM), PenStrep (1:200), B27 (without retinoic acid) (1:200), N2 (1:100), BME (1:100), NEAA (1:400), Glutamax (1:100).

VPE1 Media was prepared with DMEM-F12 as the basal media and the following reagents: SB431542 (10 uM), FGF8b (50 ng/mL), Retinoic Acid (0.1 uM), Ascorbic Acid (10 uM), Insulin-Transferrin-Selenium (ITS-G) (1:1000), KSR (0.05%), PenStrep (1:200), B27 (without vitamin A) (1:200), Glutamax (1:100), BME (1:100), N2 (1:100), NEAA (1:400).

VPE2 Media was prepared with DMEM-F12 as the basal media and the following reagents: Noggin (50 ng/mL), CHIR99021 (2 uM), FGF8b (50 ng/mL), Retinoic Acid (0.1 uM), Ascorbic Acid (10 uM), Insulin-Transferrin-Selenium (ITS-G) (1:1000), KSR (0.0025%), PenStrep (1:200), NEAA (1:400), B27 (without vitamin A) (1:200), Glutamax (1:100), BME (1:100) and N2 (1:100).

TEP Media was prepared with DMEM-F12 as the basal media and the following reagents: FGF10 (50 ng/mL), BMP4 (50 ng/mL), FGF8b (50 ng/ml), CHIR99021 (2 uM), Ascorbic Acid (10 uM), Insulin-Transferrin-Selenium (ITS-G) (1:1000), KSR (0.0025%), PenStrep (1:200), B27 (without vitamin A) (1:200), Glutamax (1:100), BME (1:100), N2 (1:100) and NEAA (1:400).

TEC Media was prepared with DMEM-F12 as the basal media and the following reagents: IL-22 (20 ng/mL), FGF10 (50 ng/mL), FGF7/KGF (50 ng/mL), RANKL/TRANCE (50 ng/mL), BMP4 (50 ng/mL), FGF8b (50 ng/mL), CHIR99021 (3 uM), Ascorbic Acid (10 uM) and Insulin-Transferrin-Selenium (ITS-G) (1:1000), KSR (0.0025%), PenStrep (1:200), B27 (without vitamin A) (1:200), Glutamax (1:100), BME (1:100), N2 (1:100) and NEAA (1:400).

TEC Freeze/Thaw (FT) Media was prepared with DMEM-F12 as the basal media and the following reagents: IL-22 (20 ng/mL), FGF10 (50 ng/mL), FGF7/KGF (50 ng/ml), RANKL/TRANCE (50 ng/ml), BMP4 (50 ng/mL), FGF8b (50 ng/mL), CHIR99021 (3 uM), Ascorbic Acid (10 uM), Insulin-Transferrin-Selenium (ITS-G) (1:1000), KSR (0.0025%), PenStrep (1:200), B27 without vitamin A (1:200), Glutamax (1:100), BME (1:100), N2 (1:100) and NEAA (1:400).

Differentiation of Non-Encapsulated Single hiPSCs into Definitive Endoderm (DE)

On Day 0, the iPSCs were thawed and disaggregated using Accutase. 3-5 million single iPS cells were placed in suspension in 6-well ultra-low adhesion plates along with Media A and ROCK inhibitor Y-27632 and incubated at 37° C. and 5% CO₂ at 70 rpm. The incubator conditions remained constant during the iPS differentiation steps described herein, and the plated cells were returned to the incubator after each differentiation step, unless expressly stated otherwise.

On Day 1, 1 mL of supernatant was removed from each well of each 6-well plate and spun down. The pelleted cells were washed with PBS and resuspended in 1 mL of pre-warmed Media A and Y-27632 before being added back to the sixth well of each plate (well #6).

On Day 2, approximately 1.8 mL of the supernatant from each well was removed and 2 mL of pre-warmed Media A (without Y-27632) was added to each well. The supernatant was spun down and the pelleted cells were washed with PBS and resuspended in 2 mL of Media A (without Y-27632) before being added to the sixth well of each plate.

On Day 3, the cells along with the supernatant were collected in a 50 ml conical tube and allowed to settle. Remaining aggregates were collected by adding 2 mL of PBS to each well and transferring the remaining cells to a new conical tube. 1 mL of fresh Media B was added to each well and the plates were returned to the incubator. The supernatant (Media A) was aspirated from the settled cells in each conical tube. 20 mL of phospho-buffered saline (PBS) was added to each tube, and the tubes were gently vortexed and spun down at 1100 rpm for 4 minutes. The cells were resuspended in the required amount of Media B (based on the number of plates) and distributed equally into the wells of each plate.

On Days 4 and 5, 1 mL supernatant was removed from each well replaced with 1 mL of pre-warmed Media B. The collected supernatant was spun down and residual cells were added to the sixth well of each plate (well #6).

Differentiation of DE Cells into Anterior Foregut Endoderm (AFE)

On Day 6, the aggregates were collected along with supernatant and allowed to settle. 1-2 mL of PBS were added to each well and the remaining aggregates were collected in a new tube. 1 mL of freshly prepared AFE media was added to each well and the plates were returned to the incubator. The supernatant was aspirated from the settled cells and 20 mL PBS was added before spinning the cells down at 1100 rpm for 4 minutes. The pelleted cells were resuspended in the required amount of AFE media and distributed equally in the six-well plates.

On Days 7-8, 1 mL of supernatant from each well was replaced with 1 mL freshly prepared AFE media. The 1 mL of supernatant from each well was spun down and removed, and the residual cells were resuspended in AFE media and added back to the last well.

### Differentiation of Anterior Foregut Endoderm (AFE) to Ventral Pharyngeal Endoderm (VPE)

On the start of day 9, the aggregates were collected with the supernatant in 50 mL conical tubes and allowed to settle. 1-2 mL of PBS were added to each well and the remaining aggregates were collected in a new tube. 1 mL of freshly prepared VPE1 media was added to each well and the plates were returned to the incubator. The supernatant was aspirated from the settled cells and 20 mL PBS was added before spinning the cells down at 1100 rpm for 4 minutes. The pelleted cells were resuspended in the required amount of VPE1 media and distributed equally in the six-well plates.

On Day 10, 1 mL of the supernatant was collected from each well and replaced with 1 mL of pre-warmed VPE1 media. The collected supernatant was spun down and removed, and the residual cells were resuspended in the required amount of VPE1 media and added to the sixth well of each plate.

On Day 11, the aggregates were collected with the supernatant in 50 mL conical tubes and allowed to settle. 1-2 mL of PBS were added to each well and the remaining aggregates were collected in a new tube. 1 mL of freshly prepared VPE2 media was added to each well and the plates were returned to the incubator. The supernatant was aspirated from the settled cells and 20 mL PBS was added before spinning the cells down at 1100 rpm for 4 minutes. The pelleted cells were resuspended in the required amount of VPE2 media and distributed equally in the six-well plates.

On Day 12, 1 mL of supernatant was collected from each well and replaced with 1 mL of pre-warmed VPE2 media. The collected supernatant was spun down and removed, and the residual cells were resuspended in the required amount of VPE2 media and added back to the sixth well of each plate.

### Differentiation of Ventral Pharyngeal Endoderm (VPE) to Thymic Epithelial Progenitors (TEP)

On Day 13, the VPE aggregates along with the supernatant were collected in 50 mL conical tubes and allowed to settle. 1-2 mL of PBS were added to each well and the remaining aggregates were collected in a new tube. 1 mL of freshly prepared TEP media was added to each well and the plates were returned to the incubator. The supernatant was aspirated from the settled cells and 20 mL PBS was added before spinning the cells down at 1100 rpm for 4 minutes. The pelleted cells were resuspended in the required amount of TEP media and distributed equally in the six-well plates.

On Days 14, 15 and 16, 1 mL of the supernatant was collected from each well and replaced with 1 mL of pre-warmed TEP media. The collected supernatant was spun down and removed, and the residual cells were resuspended in the required amount of TEP media and added to the sixth well of each plate.

On day 17, about 1.8 mL of the supernatant from each well was collected and 2 mL of pre-warmed TEC media was added in its place. The collected supernatant was spun down and removed, and the residual cells were resuspended in TEC media and added back to the last well (well #6). In cases of large numbers of aggregates, the residual cells were added back to 2 or more wells.

On day 18, 1 mL of supernatant from each well was collected and 1 mL of pre-warmed TEC media was added in its place. The collected supernatant was spun down and removed, and the residual cells were resuspended in pre-warmed TEC media and added back to the last well (well #6). This step was repeated on day 19, day 20, day 21 and day 22.

### Harvesting, Sample Collection and Freezing of Differentiated Cells

On day 23, TEP aggregates were collected and the plate was washed with DPBS to collect any remaining TEP aggregates. After letting the TEP aggregates settle, the supernatant was aspirated. The TEP aggregates were treated with Accutase to dissociate into single cells, and a sample of 0.5 to 1.0 mL was removed for cell counting and qRT-PCR.

1-2 mL of fresh TEC media and 10 mL of pre-warmed DMEM-F12 media were added to the cells, and they were spun down at 1110 rpm for 4 minutes. 10 mL of fresh TEC media was added, and the TEC cells were counted using a Cell Counter. The remaining cells were spun down and the supernatant was removed and replaced with cryopreservation media 1 (500 uL per cryopreservation vial). Cryopreservation media 2 was added dropwise to the cells (500 uL per vial) 6-10 million cells were included in each cryopreservation vial and the vials were frozen in liquid nitrogen and stored at −80° C.

### Differentiation of Thymic Epithelial Progenitors to Thymic Epithelial Cells (TEP to TEC)

On day A, the TECs were thawed and 1 mL of TEC Freeze/Thaw (FT) Media was added to each vial. The thawed cells were transferred to a conical tube and spun down at 1110 rpm for 4 minutes. The supernatant was removed and 6 mL of TEC FT media (with Y27632 at 1:1000) was added to each tube to resuspend the TECs. TEC FT media with Y27632 at 1:1000 was also added to the wells of each 6-well plate (1 mL per well), and the 1 mL of resuspended cells was added into each well. The plates were placed into the incubator (shaking at 70 rpm, 37° C., 5% CO₂).

On days B, C, D and E, 1 mL of supernatant was removed from each well of each plate and 1 mL of pre-warmed TEC FT media was added to each well. The supernatant was spun down and aspirated, and the residual cells were resuspended in TEC FT media and added back to the last well (well #6) of each plate.

On Day F, 1 mL of supernatant was removed from the well of each plate and 1 mL of pre-warmed TEC FT media with PluriSIn (20 uM) was added to each well. The supernatant was spun down and aspirated, and the residual cells were resuspended in TEC FT media with PluriSIn (20 uM) and added back to the last well (well #6).

On Day G, the TEC aggregates were collected and washed with DPBS twice. The supernatant was then aspirated and the cells were resuspended in 10 mL of DPBS. Four separate (0.5 to 1 mL) samples were collected and used for cell counting, flow cytometry, qRT-PCR and CFU analyses. For qPCR analyses, the DPBS media was removed from the corresponding 0.5-1.0 mL sample and 200-400 uL RLT buffer was added. The TEC cells in the qPCR sample were lysed and stored at −80° C. for RNA extraction. For the flow cytometry analyses, the TEC aggregates in the 0.5 to 1.0 mL sample were washed with DPBS once and spun down at 1100 rpm for 4 minutes. The supernatant was aspirated and 1-2 mL of Accutase was added to each tube. After a 4 minute incubation, the TEC aggregates were dissociated. washed with 1 mL of DPBS then spun down. The Accutase/supernatant was aspirated and DPBS was added to the cells before they were put on ice for staining.

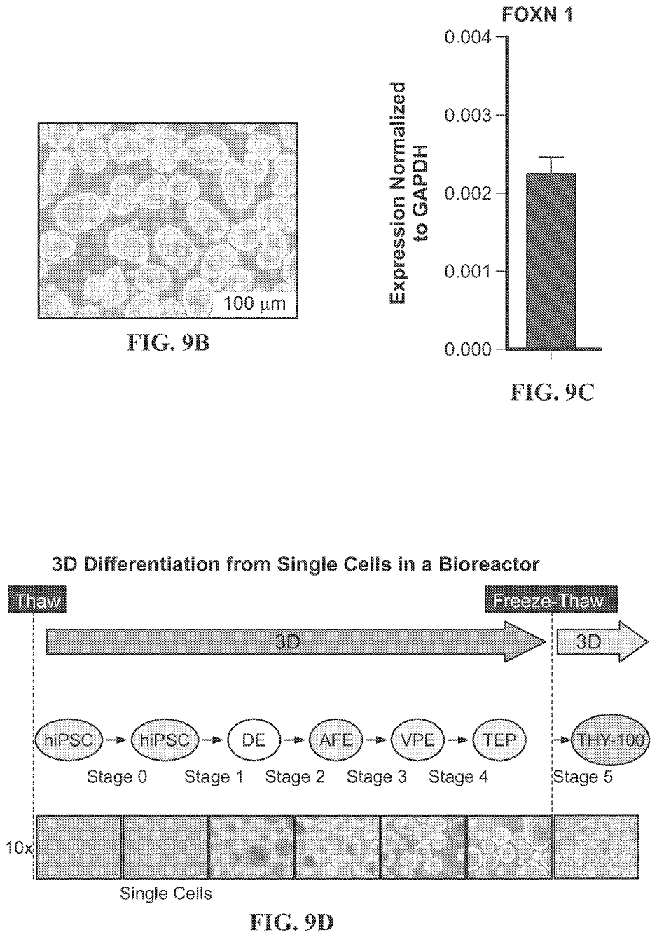

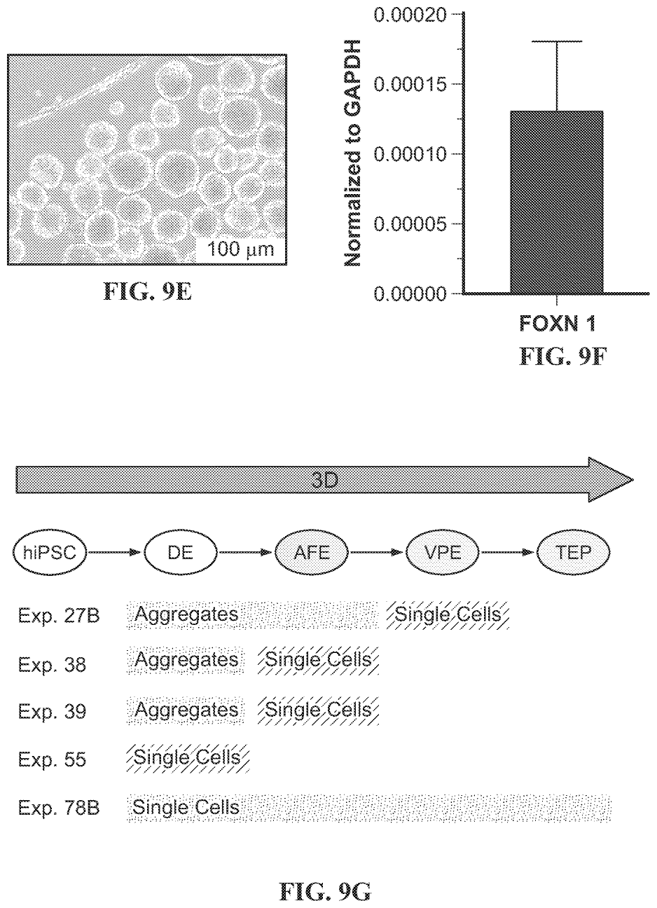

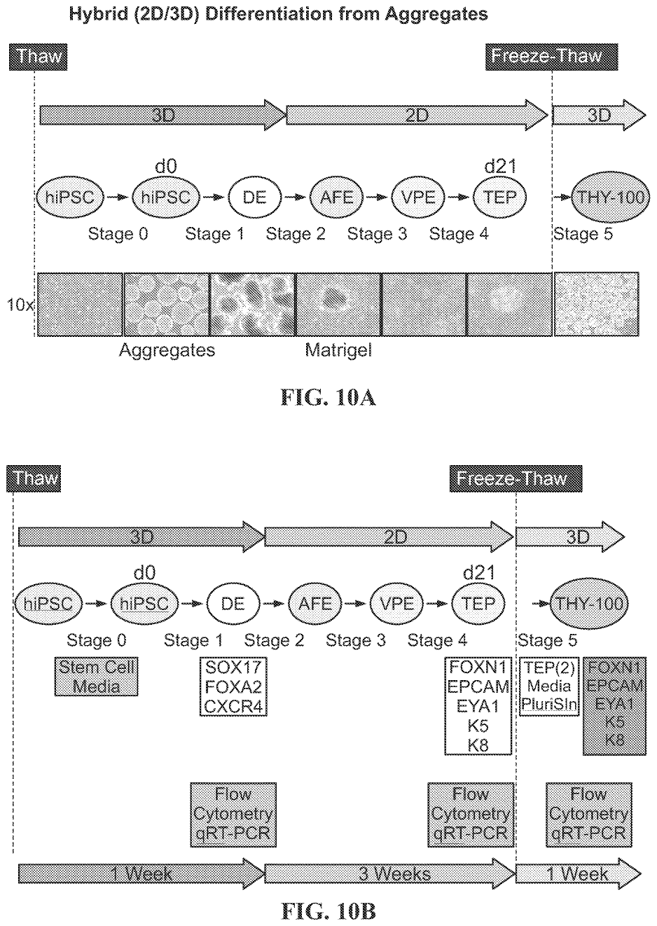

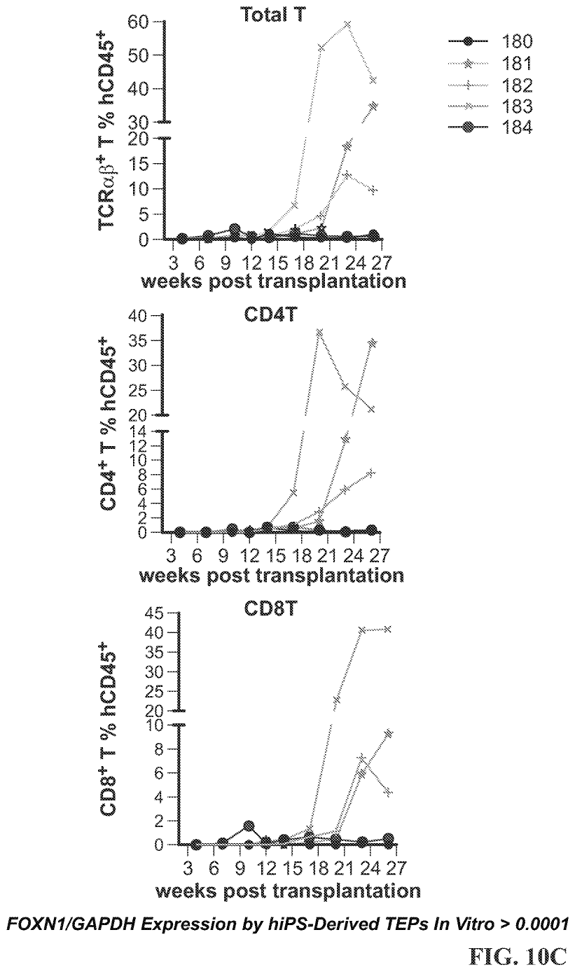

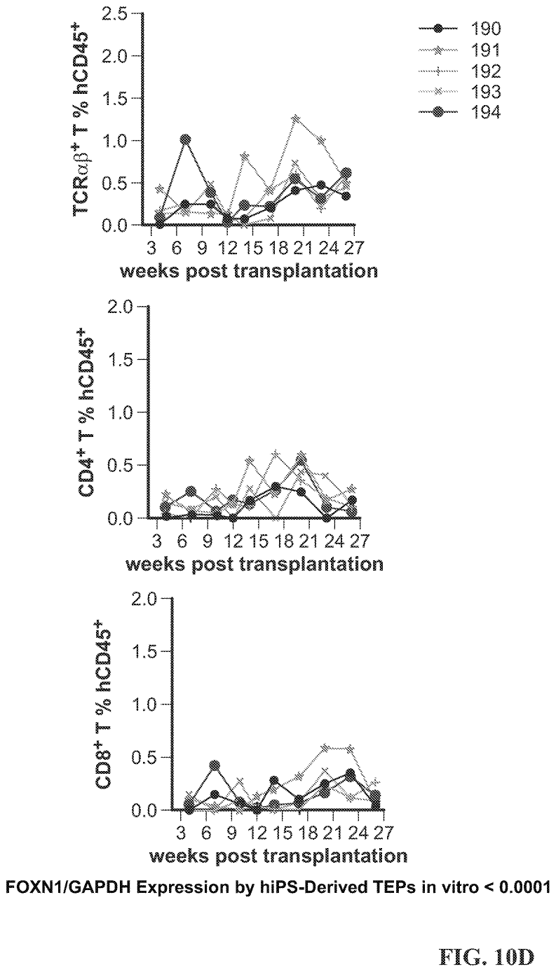

FIG. 9A illustrates the hiPSC to TEC differentiation process described in this Example, in which non-encapsulated single cells were cultured in 3D suspension. Successful growth and differentiation to thymic epithelial precursor cells was achieved, as illustrated in the representative micrographs at the bottom of FIG. 9A and in FIG. 9B (showing an enlarged version of the far right micrograph in FIG. 9A, when thymic cells were in Stage 5 of the differentiation process; scale bar=100 μm). The thymic cells produced from hiPSCs differentiated as single cells in 3D suspension culture expressed levels of FOXN1 in vitro (ranging from 0.0019 to 0.0022) which was about 19-to-22-fold higher than the threshold level (0.0001) necessary to obtain sufficient T cell production in vivo once the thymic cells were transplanted into mice as in Example 6 (as illustrated by the data in FIG. 10A). The level of FOXN1 expression (shown in FIG. 9C) was determined using qRT-PCR on thymic cell samples at a concentration of about 200,000 thymic cells per mL, with FOXN1 expression normalized to GADPH expression. As shown in FIG. 9G, non-encapsulated single-cell differentiation using 3D suspension culture was successful when B27 was added at the DE stage.

The expression levels for the following genes were also assessed for the TEP cells on Day 23, (prior to freezing) and again on Day G (after having been frozen, thawed and grown for 7 days in TEC FT media): Oct4, NANOG, HOXA3, TBX1, EYA1, EPCAM, PAX9, PAX1, FOXN1, DLL4, HLA-DRA, AIRE, CK5, CK8, FEZf2, CLDN3, CLDN4, PRSS16, LY75 and JAG2. These results also indicated that differentiated TECs display thymopoietic capacity for supporting T-cell development in vitro.

The in vitro FOXN1 expression levels when hiPSCs were differentiated as single cells in 3D in a bioreactor according to the process illustrated in FIG. 9D were also above the threshold of 0.0001, as shown in FIG. 9F.

### Example 6. Intramuscular Transplantation of Differentiated TEPs Demonstrates FOXN1 Expression Levels In Vitro Correlate with T Cell Production In Vivo

Non-encapsulated human iPSC cells were differentiated into TECs using a hybrid (2D/3D) protocol as illustrated in FIGS. 10A and 10B, using the same culture media as in Example 5.

Approximately 1 million TEP cells plus 300,000 mesenchymal stem cells were injected intramuscularly into humanized NOD/SCID/IL2Rg^{Null} (NSG) MHC-I/II double knockout mice (Jackson Laboratories).

The efficiency of thymopoiesis and generation of T cells were assessed every three weeks via flow cytometry in order to track the emergence of T cells and their immunophenotypes. Markers for human hematopoietic cells (hCD45) and human T cells (CD4, CD8 and TCRαβ) were observed, as shown in FIGS. 10C and 10D. The results shown in FIG. 10C are representative for the results obtained in similar experiments when the TECs expressed FOXN1 in vitro at a level higher than the 0.0001 threshold that was found to be necessary for obtaining high levels of T cells in vivo. Specifically, the TECs in FIG. 10C expressed FOXN1 at a level that was 8-fold higher than the threshold (0.0001). When these TECs were transplanted into NSG MCH Class I-II knockout mice, the level of total T cells, CD4+ T cells, and CD8+ T cells observed between weeks 18-26 were significantly higher for the mice that received these TECs (see, e.g., 181, 182, and 183 in FIG. 10C). In comparison, when TECs that expressed FOXN1 in vitro at a level less than the 0.0001 threshold were transplanted, the levels of total T cells, CD4+ T cells and CD8+ T cells were significantly lower. FOXN1 expression levels in vitro were determined using qRT-PCR normalized to GADPH expression. Each line (180-184 in FIG. 10C and 190-194 in FIG. 10D) represents a different mouse of the same strain.

Although the invention has been described with reference to the presently preferred embodiments, it should be understood that various modifications can be made without departing from the spirit of the invention. Accordingly, the invention is limited only by the following claims.

1. A method of differentiating pluripotent stem cells to thymic cells, comprising:
a) differentiating the pluripotent stem cells into definitive endoderm (DE) cells;
c) culturing the DE cells and differentiating the DE cells into anterior foregut endoderm (AFE) cells by contacting or incubating the DE cells with a BMP inhibitor, a TGFβ inhibitor, an FGF, an ascorbic acid or a combination thereof;
d) culturing the AFE cells and differentiating the anterior foregut cells into ventral pharyngeal endoderm (VPE) cells comprising:
i) contacting or incubating the AFE cells in a first VPE medium comprising ascorbic acid, a retinoic acid, an FGF, a TGFβ inhibitor or a combination thereof, and
ii) contacting or incubating the AFE cells in a second VPE medium comprising a Noggin, a WNT activator, an FGF, a retinoic acid, an ascorbic acid, or a combination thereof, and
e) culturing the VPE cells and differentiating the VPE cells into thymic cells, by contacting or incubating the VPE cells with an ascorbic acid, an FGF, a BMP, a WNT activator or a combination thereof;
wherein the thymic cells are thymic epithelial progenitor cells (TEPs) and/or thymic epithelial cells (TECs).

2. The method of claim 1, further comprising encapsulating the pluripotent stem cells in a polymer differentiating the pluripotent stem cells into definitive endoderm (DE) cells.

3. The method of claim 1, wherein the TEPs are further differentiated into thymic epithelial cells (TECs) by contacting or incubating the TEPs with an Interleukin, a WNT activator, a RANKL, an FGF, a BMP, an ascorbic acid, or a combination thereof.

4. The method of claim 1, wherein the differentiation of pluripotent stem cells to DE cells comprises:
a) contacting or culturing the pluripotent stem cells in a first growth medium, said first growth medium comprising Activin A, PI-103, CHIR99021 or a combination thereof; and
b) culturing the pluripotent stem cells in a second growth medium to generate definitive endoderm cells, wherein the second growth medium comprises: Activin A, a BMP inhibitor, PI-103, CHIR99021 or a combination thereof.

5. The method of claim 1, wherein;
(i) the first VPE media further comprises a WNT inhibitor; and/or
(ii) the second VPE media further comprises a BMP inhibitor, an SHH inhibitor, or a combination thereof, optionally wherein the SHH inhibitor is SANT-1.

6. (canceled)

7. The method of claim 2, wherein the polymer comprises a biogenic polymer, a synthetic polymer, a composite polymer, a hydrogel, or a cross-linked polymer, optionally wherein the polymer is alginate.

8. (canceled)

9. The method of claim 1, wherein;
(i) the BMP inhibitor is LDN193189;
(ii) the TGFβ inhibitor is SB431542;
(iii) the FGF is FGF8b, FGF7, FGF10, FGF1, bFGF or a combination thereof;
(iv) the WNT activator is CHIR99021; and/or
(v) the BMP is BMP2, BMP4 or a combination thereof.

10-13. (canceled)

14. The method of claim 3, wherein;
(i) the interleukin is IL22;
(ii) the WNT inhibitor is IWR-1; and/or
(iii) the BMP is BMP2, BMP4 or a combination thereof.

15-17. (canceled)

18. The method of claim 1, wherein the pluripotent stem cells, the DE cells, the AFE cells, the VPE cells or thymic cells are cultured in 3D culture or cultured as aggregates in suspension.

19. (canceled)

20. The method of claim 1, wherein the method is performed for from about 15 days to 30 days, optionally, wherein the method is performed for from about 18 days to 25 days.

21. (canceled)

22. The method of claim 1, wherein;
(i) the pluripotent stem cells are differentiated into definitive endoderm cells for about 5 days;
(ii) the DE cells are differentiated into AFE cells for from about 2 days to 3 days;
(iii) the AFE cells are cultured in the first VPE media for about 2 to 4 days;
(iv) the AFE cells are cultured in the second VPE media for about 2 to 3 days; and or
(v) the VPE cells are differentiated to thymic cells for about 3 to 6 days, optionally wherein the TEPs are differentiated to TECs for about 4 days.

23-27. (canceled)

28. The method of claim 2, wherein the polymer is removed from the TEPs or wherein the polymer is removed from the TECs.

29-33. (canceled)

34. A composition comprising:
a) a population of thymic cells, wherein the population of thymic cells comprises one or more of an iTEC, mTECs, Corneocyte-like mTEC, cTEC-high, cTEC-low, mTEC-low cell; and
b) a thymic support system (TSS).

35. (canceled)

36. The composition of claim 34, wherein the TSS comprises a polymer, optionally wherein the polymer is a biogenic polymer or a synthetic polymer.

37. (canceled)

38. The composition of claim 36, wherein: the polymer is a biogenic polymer, and wherein the biogenic polymer is a polypeptide-based biogenic polymer, a polynucleotide-based biogenic polymer, or a polysaccharide-based polymer;
optionally wherein (i) the polymer is a polypeptide-based polymer, said polypeptide-based polymer selected from the group consisting of collagen, fibrin, fibrinogen, gelatin, silk, elastin, myosin, keratin, and actin; or
(ii) the polymer is a polysaccharide-based polymer, said polysaccharide-based polymer selected from the group consisting of chitin, chitosan, alginate, hyaluronic acid, cellulose, agarose, starch, cellulose, dextran, hyaluronic acid, glycogen and glycosaminoglycans, or
wherein the polymer is a synthetic polymer, said synthetic polymer selected from the group consisting of Polycaprolactone, polyglycolic acid, poly lactic acid, polylactic-co-glycolic acid, poly(ethylene oxide) polyethylene glycol, polyurethane,), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), polyhydroxybutyrate (PHB), Polypropylene fumarate (PPF), a polyvinyl alcohol (PVA), a polypropylene carbonate, a polyanhydride, a polyphosphazene, a polygermane, a polyorthoester, a polyester, a polyamide, a polyolefin, a polycarbonate, a polyaramide, a polyimide,), chitosan, Poly(2-hydroxyethyl methacrylate) (PHEMA), 2-Hydroxyethyl methacrylate (HEMA), Hydroxyethoxyethyl metha-crylate (HEEMA), Hydroxydiethoxyethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl Aam (NIPAAm), Vinyl acetate (Vac), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic poly allyl ammonium chloride, and poly(N-isopropyl acrylamide) (PNIPAM).

39-41. (canceled)

42. The composition of claim 34, wherein the polymer forms a hydrogel, optionally wherein the polymer is cross-linked.

43. (canceled)

44. The composition of claim 34, wherein the TSS further comprises one or more of an extracellular matrix component and an agent,
wherein the extracellular matrix component is an extracellular matrix protein, a peptide derived from the extracellular matrix protein or a region or a portion thereof, optionally wherein the peptide comprises the amino acid sequence of SEQ ID NO: 9-18 optionally wherein the extracellular matrix protein is fibronectin, laminin, vitronectin, tenascin, entactin, thrombospondin, elastin, gelatin, collagen, fibrin, merosin, ankaline, chondronectin, link protein, bone sialo acid protein, osteocalcin, osteopontin, epinectin, hyaluronectin, unjulin, Epiligrin or carinine, and/or
wherein the agent is a biological agent or a chemical agent, optionally wherein the biological agent is a ligand, an immune modulator, or a hormone.

45-50. (canceled)

51. The composition of claim 34, further comprising (i) a population of supporting cells, said supporting cells comprising one or more of a myelin cell, a myoid cell, a neuroendocrine cell, a Tuft cell, an ionocyte, an endothelial cell, a mesenchymal stem cell, or a fibroblast, (ii) a population of effector cells and/or (iii) a population of stem cells.

52-53. (canceled)

54. The composition of claim 30, wherein the level of FOXN1 production in vitro by a sample of the thymic cell population having a concentration of about 200,000 thymic cells per milliliter is greater than 0.0001 when measured by qRT-PCR and normalized against GADPH.

55. A method of treating or preventing a condition in a subject comprising administering to the subject the composition of as in claim 30.

56. The method of claim 55, wherein the condition is associated with the absence, decline or aberrant functioning of the thymus of the subject, such as Di George syndrome, thymoma (such as type A thymoma or type B thymoma), CHARGE syndrome, FOXN1 deficiency, PAX1 deficiency, TBX1 deficiency, thymus cancer, thymic atrophy (such as age-related thymic atrophy), thymic cyst, thymic hyperplasia, thymic hypoplasia, thymic aplasia, thymic dysplasia, thymic irradiation, myasthenia gravis, thymic carcinoma, thymic hyperplasia, thymic irradiation, or age- or infection-associated decline in thymic function, optionally wherein the subject has undergone a thymectomy surgery.

57. (canceled)

58. The method of claim 55, wherein the condition is selected from the group consisting of immunodeficiency, cancer, autoimmune disease, infectious disease, and graft versus host disease (GvHD).

59-62. (canceled)

63. A method of improving acceptance of an organ transplant and/or preventing rejection of an organ transplant in a subject receiving an organ transplant comprising administering to the subject the composition as in of claim 30.
